(19) United States
(12) Patent Application Publication
(10) Pub. No.: US 2022/0378058 A1
(43) Pub. Date: Dec. 1, 2022

(54) COMPOSITIONS COMPRISING SUBSETS OF MILK LIPIDS, AND METHODS FOR PRODUCING THE SAME

(71) Applicant: PERFECT DAY, INC., BERKELEY, CA (US)

(72) Inventors: PERUMAL GHANDI, Berkeley, CA (US); RAVIRAJSINH JHALA, Castro Valley, CA (US); RYAN PANDYA, Berkeley, CA (US); TIMOTHY GEISTLINGER, Oakland, CA (US)

(21) Appl. No.: 17/642,174

(22) PCT Filed: Sep. 10, 2020
(86) PCT No.: PCT/US2020/050241

§ 371 Date: Mar. 10, 2022

Related U.S. Application Data
(60) Provisional application No. 62/898,436, filed on Sep. 10, 2019.
(60) Provisional application No. 62/984,891, filed on Mar. 4, 2020.

(51) Int. Cl. A23C 9/13; A23C 9/152; A23C 11/02; A23J 3/08

(52) U.S. Cl. CPC A23C 9/1307; A23C 9/1528; A23C 11/02; A23J 3/08

(57) ABSTRACT

The present invention relates generally to a lipid component that comprises a subset of milk lipids and that can confer on a composition an attribute conferred by a mammal-produced milk or milk fat. The present invention further provides methods for producing such lipid component. The present invention further relates to compositions that comprise such lipid component, and methods for producing the same.

### FIELD OF THE INVENTION

The present invention relates generally to a milk lipid component that comprises a subset of milk lipids that are normally found in a mammal-derived milk or milk fat, and that can confer on a composition an attribute conferred by a mammal-produced milk or milk fat. The present invention further provides methods for producing such milk lipid component. The present invention further relates to compositions and products that comprise such milk lipid component, and methods for producing the same.

### BACKGROUND OF THE INVENTION

Milk fat is one of the most complex natural animal fats. In cows, it makes up between 3% and 6% of cow's milk, and comprises more than 400 different saturated and unsaturated fatty acids, and more than 1,000 different milk triglycerides (mTAGs). It further comprises milk phospholipids (mPLs), milk diglycerides (mDAGs), milk monoglycerides (mMAGs), sterols, carotenoids, fat-soluble vitamins (e.g., vitamins A, vitamin D), fat-soluble flavor compounds, and milk free fatty acids (mFFAs). The mTAGs, mDAGs, mMAGs, mPLs, and mFFAs comprise a uniquely high content of short-chain fatty acids.

The unique composition of milk fat is believed to give rise to unique attributes (e.g., physical, chemical/biological, sensory, and functional attributes). For example, mTAGs contribute to nutritive content, mouthfeel (e.g., creaminess, lubricity), texture, and melting behavior; mPLs have superior emulsifying properties; and the short-chain mFFAs and fatty acids released from mTAGs contribute to distinct flavor/aroma profiles.

Various motivations have led to a search for a substitute for milk fat. Among such motivations are concerns for animal welfare, and the deleterious impact of animal farming on the environment. It is estimated that 30% of Earth's land surface is dedicated to animal farming, and that livestock account for 20% of total terrestrial animal biomass. Due to its massive scale, animal farming produces more than 18% of net greenhouse gas emissions, constitutes the largest human source of water pollution, and poses the world's largest threat to biodiversity. Moreover, milk fat comprises components that are difficult to remove from milk fat (e.g., cholesterol) but that can negatively impact human health.

The search for a substitute for milk fat has explored, for example, the use of vegetable fat in place of milk fat. However, most of the vegetable fats tested to date do not match the functionality, flavor, flavor stability, and physical properties (e.g., crystallization, melting profile) of milk fat. Such failure may, in part, be due to plant fats not comprising short-chain fatty acids like butyric and caproic acid, not comprising lipid-soluble vitamins like vitamins A and D, and not having the nutritional profile and physical attributes (e.g., thermal profile, solid fat content at various temperatures, crystallization behavior, oxidation stability) of milk fat.

There have also been publications of lipids obtained from microbial sources such as bacteria, yeast, and algae. However, many of these microbial lipids are rich in long-chain fatty acids, making them suitable for synthesis of biofuels but not for replacing milk fat with its high content of short-chain fatty acids.

Therefore, there exists a need for milk fat substitutes, and compositions comprising such milk fat substitutes, and methods for producing the same.

### INCORPORATION BY REFERENCE

All publications, patents, patent applications, sequences, database entries, scientific publications, and other references mentioned herein are incorporated by reference in their entireties to the same extent as if each individual publication, patent, patent application, sequence, database entry, scientific publication, or other reference was specifically and individually indicated to be incorporated by reference. To the extent the material incorporated by reference contradicts or is inconsistent with the present disclosure, the present disclosure, including definitions, will supersede any such material.

### SUMMARY OF THE INVENTION

In one aspect, provided herein is a lipid component, wherein the lipid component consists of a milk lipid component and an optional non-milk lipid component. The milk lipid component can consist of a single milk lipid or of two or more milk lipids. The single milk lipid or two or more milk lipids can consist of or comprise, for example, one or more mTAGs, one or more mDAGs, one or more mMAGs, one or more mPLs, one or more milk sterols, one or more mFFAs, or any combination thereof. The single milk lipid or two or more milk lipids can consist of or comprise, for example, one or more bovine milk lipids, one or more sheep milk lipids, one or more goat milk lipids, one or more human milk lipids, or any combination thereof. The milk lipids comprised in the milk lipid component according to any of the above can be present at relative ratios found in a mammal-produced milk or milk fat, or at relative ratios not found in a mammal-produced milk or milk fat. The milk lipids comprised in the milk lipid component according to any of the above can have a fatty acid profile that is identical or similar to the fatty acid profile of a mammal-produced milk or milk fat, or a fatty acid profile that is different from the fatty acid profile of a mammal-produced milk or milk fat. The single milk lipid or two or more milk lipids comprised in a lipid component according to any of the above can consist of or comprise one or more structured milk lipids. The lipid component according to any of the above can an identical or similar melting profile, flavor/aroma profile, and/or emulsifying potential as a mammal-produced milk or milk fat; and/or impart on a composition an identical or similar attribute as one that can be imparted by a mammal-produced milk or milk fat (e.g., a physical attribute, chemical/biological attribute, sensory attribute, functional attribute, and any combination thereof).

In another aspect, provided herein is a composition that comprises a lipid component according to any of the above, wherein the composition comprises no other lipid than the lipids of which the lipid component consists. The composition can further optionally comprise a milk protein component, a non-milk protein component, a milk fat globule-like structure component (e.g., comprising milk globule-like structures that comprise a milk lipid and a milk protein), and/or an other ingredient. The other ingredient can be a flavor/aroma agent, such as, for example, a milk volatile organic compound or a green leaf volatile organic compound obtained by chemical and/or enzymatic modification of a milk lipid (e.g., by chemical and/or enzymatic degradation of a plant lipid, or by fermentation of a milk lipid). The composition according to any of the above can be a fluid, a semi-solid, a solid, a powder, or an emulsion (e.g., an emulsion comprising dispersed phase droplets that are engulfed in a membrane). The composition according to any of the above can a food product (e.g., an egg, an egg product, an egg substitute, an egg product substitute, a milk, a dairy product, a milk substitute, a dairy product substitute, an animal meat, an animal meat product, an animal meat substitute, an animal meat product substitute).

In another aspect, provided herein is a method for producing a lipid component according to any of the above, wherein the method comprises the step of obtaining a milk lipid or milk lipid precursor. The milk lipid or milk lipid precursor can be obtained, for example, by chemical or enzymatic modification of a milk lipid precursor (e.g., chemical or enzymatic inter-esterification), by fermentation of biomass, and/or by culturing a recombinant host cell capable of producing the milk lipid or milk lipid precursor under conditions suitable for production of the milk lipid or milk lipid precursor.

In another aspect, provided herein is a recombinant host cell that is capable of producing a milk lipid or a milk lipid precursor, wherein the recombinant host cell comprises one or more genetic modifications that essentially eliminate or modulate production and/or activity of one or more lipid biosynthesis-related proteins. Non-limiting examples of lipid biosynthesis-related proteins include: enzymes with activity in the production of unsaturated fatty acids; enzymes with activity in the production of fatty acids having a carbon atom number of greater than 16; enzymes with activity in the production of fatty acids having a carbon atom number of 16 or less; enzymes with activity in the b-oxidation pathway or peroxisome biogenesis; enzymes with activity in the production of cytosolic acetyl-CoA; enzymes with activity in the production of a TAG, DAG, MAG, and/or PL; enzymes with activity in the production of an amino acid; enzymes with activity in the production of cytosolic NADPH; enzymes with activity in inter-esterification or trans-esterification; and any combination of one or more such enzymes.

The recombinant host cell according to any of the above can be a recombinant plant cell, a recombinant animal cell, or a recombinant microbial cell. The recombinant host cell according to any of the above can be a recombinant oleaginous cell.

In another aspect, provided herein is a method for producing a food product. The method can comprise the step of combining a lipid component according to any of the above with other ingredients and/or the step of fermenting the lipid component.

### DETAILED DESCRIPTION OF THE INVENTION

The subsequent discussion of the invention is presented for purposes of illustration and description, and is not intended to limit the invention to the embodiments disclosed herein. As such, variations and modifications of the disclosed embodiments are within the scope of the invention, e.g., as may be within the skill and knowledge of those in the art after understanding the present disclosure. It is intended to obtain rights which include alternative embodiments to the extent permitted, including alternate, interchangeable and/or equivalent structures, functions, ranges or steps to those disclosed herein, and without intending to publicly dedicate any patentable subject matter.

Unless defined otherwise, all technical and scientific terms used herein have the same meaning as is commonly understood by one of ordinary skill in the art to which this disclosure pertains. Further, unless otherwise required by context, singular terms shall include the plural, and plural terms shall include the singular.

### Definitions

The terms “a” and “an” and “the” and similar references as used herein refer to both the singular and the plural (e.g., meaning “at least one” or “one or more”), unless otherwise indicated herein or clearly contradicted by context. For example, the term “a compound” or “at least one compound” or “one or more compounds” may include a plurality of compounds, including mixtures thereof.

The term “about” as used herein in conjunction with a stated numerical value or range of numerical values is meant to encompass variations of the stated numerical value or range of numerical values (i.e., denoting somewhat more or somewhat less than the stated numerical value or range of numerical values, to within a range of 20%, +10%, ±5%, +1%, ±0.5%, +0.1%, or one standard deviation of the stated value or range of numerical values).

The term “and/or” as used herein refers to multiple components in combination with or exclusive of one another. For example, “x, y, and/or z” can refer to “x” alone, “y” alone, “z” alone, “x, y, and z”, “(x and y) or z”, “(x and z) or y”, “(y and z) or x”, “x and y” alone, “x and z” alone, “y and z” alone, or “x or y or z”.

The term “aroma compound” as used herein refers to a volatile substance that activates aroma receptors in the olfactory system.

The term “aroma” as used herein refers to the smell or odor that represents the sensory attributes of certain volatile substances perceptibly by the olfactory system (i.e., the main and accessory olfactory organs). The aroma of a food product can be tested using a panel of expert human subjects. Alternatively, the aroma of a food product can be tested by head space gas chromatography-mass spectrometry (GCMS), including using automated olfactometers, such as, for example, the Heracles II (Alpha MOS America, Hanover, Md.).

The term “component” as used herein refers to one or more agents that are grouped together. The grouping is to be understood as only a symbolic grouping, and thus does not require physical interaction between the agents (although physical interaction is not ruled out by the use of the term “component”).

The term “encoding” as used herein refers to a polynucleotide that comprises a coding sequence that when placed under the control of appropriate regulatory sequences is transcribed into mRNA that can be translated into a polypeptide. A coding sequence generally starts at a start codon (e.g., ATG) and ends at a stop codon (e.g., UAA, UAG and UGA). A coding sequence may contain a single open reading frame, or several open reading frames (e.g., separated by introns).

The term “endogenous” as used herein refers to what is natively present in the context described. When used in reference to a protein that is produced by a cell, the term implies that the protein is natively produced by the cell. When used in reference to a polynucleotide that is comprised in a cell, the term implies that the polynucleotide is natively comprised in the cell (e.g., is present in the native cell; or is situated in the same genomic location in the native cell).

The term “essentially free of” as used herein refers to the indicated component being either not detectable in the indicated composition by common analytical methods, or being present in such trace amount as to not be functional. The term “functional” as used in this context refers to not contributing to properties of the composition comprising the trace amount of the indicated component, or to not having activity (e.g., chemical activity, enzymatic activity) in the indicated composition comprising the trace amount of the indicated component, or to not having health-adverse effects upon use or consumption of the composition comprising the trace amount of the indicated component.

The term “fat” as used herein refers to a lipid composition that is solid at ambient conditions (i.e., 20° C.-30° C. and 0.95-1.05 atm).

The term “fatty acid” as used herein refers to both a fatty acid and a fatty acyl group without reference to attachment to a glycerol backbone or reference to the regiospecific nature of any connection to a glycerol backbone.

The term “fatty acid profile” as used herein refers to the distribution of fatty acids (e.g., distribution of types of fatty acids and/or abundances of distinct types of fatty acids and/or relative amounts of distinct types of fatty acids) in a composition without reference to attachment to a glycerol backbone or reference to the regiospecific nature of any connection to a glycerol backbone. Fatty acid profiles are typically determined by conversion to a fatty acid methyl ester (FAME), followed by gas chromatography (GC) analysis with flame ionization detection (FID). A fatty acid profile can be expressed as percent of a fatty acid in a total fatty acid signal determined from the area under the curve for that fatty acid.

The term “filamentous fungus” as used herein refers to any filamentous form of the subdivision Eumycota and Oomycota (as defined by Hawksworth et al., In, Ainsworth and Bisby's Dictionary of The Fungi, 8th edition, 1995, CAB International, University Press, Cambridge, UK). A filamentous fungus is distinguished from yeast by its hyphal elongation during vegetative growth. The term “filamentous fungal cell” as used herein refers to a cell that is obtained from a filamentous fungus.

The term “food product” as used herein refers to a composition that can be ingested by a human or an animal for dietary purposes (i.e., without ill health effects but with significant nutritional and/or caloric intake due to uptake of digested material in the gastrointestinal tract), including a domesticated animal (e.g., dog, cat), farm animal (e.g., cow, pig, horse), and wild animal (e.g., non-domesticated predatory animal). The term includes compositions that can be combined with or added to one or more other ingredients to make a food product that can be ingested by a human or an animal.

The term “free fatty acid” as used herein refers to a fatty acid that is not bound to a glycerol backbone.

The term “fungus” as used herein refers to an organism of the phyla Ascomycotas, Basidiomycota, Zygomycota, and Chythridiomycota, Oomycota, or Glomeromycota. It is understood, however, that fungal taxonomy is continually evolving, and therefore this specific definition of the fungal kingdom may be adjusted in the future. The term “fungal cell” as used herein refers to a cell that is obtained from a fungus.

The term “glycerolipid” as used herein refers to a lipid that consists of a glycerol backbone and between 1 and 3 fatty acids (which can be of varying lengths and have varying degrees of saturation) that are attached to the glycerol backbone via ester bonds. The term includes monoglycerides, diglycerides, triglycerides, and phospholipids.

The term “glycerolipid profile” as used herein refers to the distribution of glycerolipids (e.g., monoglycerides, diglycerides, triglycerides, phospholipids) in a composition.

The term “heterologous” as used herein refers to not being natively present in the context described. When used in reference to a protein that is produced by a cell, the term implies that the protein is not natively produced by the cell. When used in reference to a polynucleotide that is comprised in a cell, the term implies that the polynucleotide is not natively comprised in the cell (e.g., is not present in the native cell; or is not situated in the genomic location in the native cell, whether or not the heterologous polynucleotide is itself endogenous (originating from the same cell or progeny thereof) or exogenous (originating from a different cell or progeny thereof)).

The term “homolog” as used herein refers to a protein that comprises an amino acid sequence that is at least 50% (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100%) identical to a sequence of amino acids of a similar length (i.e., a length that is within +/−20% of the length of the query amino acid sequence) comprised in a reference protein, and that has a functional property that is similar to that of the reference protein.

The term “host cell” as used herein refers not only to the particular subject cell but to the progeny of such cell. Because certain modifications may occur in succeeding generations due to either mutation or environmental influences, such progeny may not, in fact, be identical to the subject cell, but are still included within the scope of the term “host cell” as used herein.

The terms “identity” or “identical” in the context of two or more polynucleotide or polypeptide sequences as used herein refer to the nucleotide or amino acid residues that are the same when the two or more polynucleotide or polypeptide sequences, respectively, are aligned for maximum correspondence. Depending on the application, the “identity” can exist over a region of the sequences being compared (e.g., over the length of a functional domain) or over the full length of the sequences. A “region” is considered to be a continuous stretch of at least 9, 14, 19, 24, 29, 34, 39, or more nucleotides, or of at least 6, 10, 14, 18, 22, 26, 30, or more amino acids. For comparison, typically one sequence acts as a reference sequence to which one or more test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Sequence Analysis Software Package of the Genetics Computer Group (GCG), University of Wisconsin Biotechnology Center, which can be used with default parameters), or by visual inspection (see generally Ausubel et al., infra). One example of an algorithm that is suitable for determining percent sequence identity and sequence similarity is the BLAST algorithm (see, for example, Altschul et al. [1990] J. Mol. Biol. 215:403-410; Gish & States. [1993] Nature Genet. 3:266-272; Madden et al. [1996] Meth. Enzymol. 266:131-141; Altschul et al. [1997] Nucleic Acids Res. 25:3389-3402; Zhang 7 Madden. [1997] Genome Res. 7:649-656). Software for performing BLAST analyses is publicly available through the National Center for Biotechnology Information. In cases where two or more polypeptide sequences differ from each other by conservative substitutions, the percent sequence identity or degree of homology can be adjusted upwards to correct for the conservative nature of the substitution. Means for making this adjustment are well known to those of skill in the art. See, e.g., Pearson, 1994, Methods Mol. Biol. 24:307-31 and 25:365-89.

The terms “including,” “includes,” “having,” “has,” “with,” or variants thereof as used herein are intended to be inclusive in a manner similar to the term “comprising”.

The term “lipid” as used herein refers to an organic compound that is soluble in nonpolar solvents (such as ether and chloroform) and are relatively or completely insoluble in water. Non-limiting examples of lipids include glycerolipids (e.g., monoglycerides, diglycerides, triglycerides, neutral fats, phosphoglycerides, glycerophospholipids), nonglycerides (e.g., sphingolipids, sterol lipids [e.g., cholesterol, steroid hormones), prenol lipids [e.g., terpenoids], fatty alcohols, fatty acids, waxes, polyketides), and complex lipid derivatives (e.g., sugar-linked lipids, glycolipids, protein-linked lipids).

The term “mammal-produced milk” as used herein refers to a milk produced by a mammal. Non-limiting examples of mammals include cow, human, sheep, goat, water buffalo, camel, horse, donkey, lemur, panda, guinea pig, squirrel, bear, macaque, gorilla, chimpanzee, mountain goat, monkey, ape, cat, dog, wallaby, rat, mouse, elephant, opossum, rabbit, whale, baboons, gibbons, orangutan, mandrill, pig, wolf, fox, lion, tiger, reindeer, echidna, and woolly mammoth.

The term “microbe” as used herein is an abbreviation for microorganism, and refers to a unicellular organism. As used herein, the term includes all yeast, bacteria, archaea, unicellular protista, unicellular animals, unicellular plants, unicellular fungi, unicellular algae, protozoa, and chromista. The term “microbial” as used herein is the corresponding adjective.

The term “milk fat” as used herein refers to the collection of all milk lipids comprised in a mammal-produced milk.

The term “milk lipid” as used herein refers to a lipid that is natively present in a mammal-produced milk or milk fat (e.g., a mTAG, mDAG, mMAG, mPL, mFFA, milk sterol).

The term “milk lipid precursor” as used herein refers to a molecule that can be converted into a milk lipid (e.g., any of the milk lipids disclosed herein). Non-limiting examples of milk lipid precursors include glycerol, acetyl-CoA, triglycerides, diglycerides, monoglycerides, phospholipids, and free fatty acids.

The terms “milk monoglyceride”, “milk diglyceride”, “milk triglyceride”, “milk phospholipid”, and “milk free fatty acid”, and their acronyms “mMAG”, “mDAG”, “mTAG”, “mPL”, and “mFFA”, respectively, as used herein refer to a monoglyceride, diglyceride, triglyceride, phospholipid, and free fatty acid, respectively, that are natively present in a mammal-produced milk or milk fat.

The term “mono-unsaturated” as used herein refers to comprising a single carbon-carbon double or triple bond.

The term “mouthfeel” as used herein refers to the overall appeal of a food product that is independent of taste, which stems from the combination of several characteristics that together provide a satisfactory sensory experience. The mouthfeel of a food product can be determined using a panel of human sensory experts. Non-limiting examples of attributes that determine mouthfeel include creaminess, richness, body, complexity, body-richness, thickness, sliminess, and stringiness.

The term “native” as used herein refers to what is found in nature in its unmodified state.

The term “natural” or “naturally occurring” as used herein refers to what is found in nature.

The term “non-animal” as used herein refers to a component (e.g., protein, lipid, carbohydrate) that is not native to an animal cell.

The term “non-milk lipid” as used herein refers to a lipid that is not natively present in a mammal-produced milk or milk fat.

The terms “non-milk monoglyceride”, “non-milk diglyceride”, “non-milk triglyceride”, “non-milk phospholipid”, and “non-milk free fatty acid”, and their acronyms “nmMAG”, “nmDAG”, “nmTAG”, “nmPL”, and “nmFFA”, as used herein refer to a monoglyceride, diglyceride, triglyceride, phospholipid, and free fatty acid, respectively, that are natively not present in a mammal-produced milk or milk fat.

The term “one or more” as used herein refers to one, two, three, four, five, six, seven, eight, nine, ten, at least one, at least two, at least three, at least four, at least five, at least six, at least seven, at least eight, at least nine, at least ten, or more, or all of the elements subsequently listed.

The term “oil” as used herein refers to a lipid composition that is liquid at ambient conditions (i.e., 20° C.-30° C. and 0.95-1.05 atm).

The terms “optional” or “optionally” as used herein refer to a feature or structure being present or not, or an event or circumstance occurring or not. The description includes instances in which a feature or structure is present, instances in which the feature or structure is absent, instances in which an event or circumstance occurs, and instances in which an event or circumstance does not occur.

The term “plant” us used herein refers to any part of a plant, including, for example, seeds, roots, leaves, stem, xylem, phloem, cutical, cell wall, and sap.

The term “polynucleotide” as used herein refers to a polymeric form of at least 2 (e.g., at least 5, at least 10, at least 20, at least 30, at least 40, at least 50, at least 100, at least 500, at least 1,000) nucleotides. The term includes both sense and antisense strands of DNA molecules (e.g., cDNA, genomic DNA, synthetic DNA) and RNA molecules (e.g., mRNA, synthetic RNA), as well as analogs of DNA or RNA containing non-natural nucleotide analogs, non-native internucleoside bonds, and/or chemical modifications. A polynucleotide may be modified chemically or biochemically or may contain non-natural or derivatized nucleotide bases. Such modifications include, for example, labels; methylation; substitution of one or more of the naturally occurring nucleotides with an analog; internucleotide modifications such as uncharged linkages (e.g., methyl phosphonates, phosphotriesters, phosphoramidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Examples of modified nucleotides are described in the art (see, for example, Malyshev et al. 2014. Nature 509:385; Li et al. 2014. J. Am. Chem. Soc. 136:826). Also included are synthetic molecules that mimic polynucleotides in their ability to bind to a designated sequence via hydrogen bonding or other chemical interaction. Such molecules are known in the art and include, for example, molecules in which peptide linkages substitute for phosphate linkages in the backbone of the molecule. Other modifications can include, for example, analogs in which the ribose ring contains a bridging moiety or other structure such as the modifications found in “locked” polynucleotides. A polynucleotide can be in any topological conformation. For instance, a polynucleotide can be single-stranded, double-stranded, triple-stranded, quadruplexed, partially double-stranded, branched, hairpinned, circular, or in a padlocked conformation. The term “polynucleotide sequence” as used herein refers to a sequence of nucleotides that are comprised in a polynucleotide or of which a polynucleotide consists.

The terms “polypeptide” and “protein” as used herein can be interchanged, and refer to both a naturally-occurring and a non-naturally occurring polymeric form of at least 2 (e.g., at least 5, at least 10, at least 20, at least 30, at least 40, at least 50, at least 100) amino acids (e.g., coded or non-coded amino acids), having an active structure or lacking functional structure. The “polypeptide” or “protein” can have any length, comprise amino acids that occur in nature and those that do not occur in nature, comprise chemically or biochemically modified or derivatized amino acids, and/or comprise a modified peptide backbone. A protein can be monomeric, meaning having a single chain, or polymeric, meaning comprising two or more chains that are covalently or non-covalently associated. The term “polypeptide sequence” or “protein sequence” as used herein refers to a sequence of amino acids that is comprised in a polypeptide or protein, respectively, or of which a polypeptide or protein, respectively, consists.

The term “polyunsaturated fatty acid” as used herein refers to a fatty acid that contains more than one double bond. The term encompasses a fatty acid that comprises a conjugated double bond.

The term “post-translational modification”, or its acronym “PTM”, as used herein refers to the covalent attachment of a chemical group to a polypeptide after biosynthesis. A PTM can occur on the amino acid side chain of the polypeptide or at its C- or N-termini. Non-limiting examples of PTMs include glycosylation (i.e., covalent attachment to proteins of glycan groups (i.e., monosaccharides, disaccharides, polysaccharides, linear glycans, branched glycans, glycans with galf residues, glycans with sulfate and/or phosphate residues, D-glucose, D-galactose, D-mannose, L-fucose, N-acetyl-D-galactose amine, N-acetyl-D-glucose amine, N-acetyl-D-neuraminic acid, galactofuranose, phosphodiesters, N-acetylglucosamine, N-acetylgalactosamine, sialic acid, and combinations thereof, see, for example, Deshpande et al. 2008. Glycobiology 18(8):626) via C-linkage (i.e., C-glycosylation), N-linkage (i.e., N-glycosylation), or O-linkage (i.e., O-glycosylation), or via glypiation (i.e., addition of a glycosylphosphatidylinositol anchor) or phosphoglycosylation (i.e., linked through the phosphate of a phospho-serine)), phosphorylation (i.e., covalent attachment to proteins of phosphate groups), alkylation (i.e., covalent attachment to proteins of alkane groups (e.g., methane group in methylation)), and lipidation (i.e., covalent attachment of a lipid group (e.g., isoprenoid group in prenylation and isoprenylation (e.g., famesol group in farnesylation, geraniol group in geranylation, geranylgeraniol group in geranylgeranylation), fatty acid group in fatty acylation (e.g., myristic acid in myristoylation, palmitic acid in palmitoylation), glycosylphosphatidylinositol anchor in glypiation)), hydroxylation (i.e., covalent attachment of a hydroxide group), sumoylation (i.e., attachment to proteins of Small Ubiquitin-like Modifier (or SUMO) protein), nitrosylation (i.e., attachment to proteins of an NO group; e.g., S-nitrosylation (i.e., attachment to a cysteine thiol in a protein of an NO group to form an S-nitrosothiol)), S-glutathionylation (i.e., attachment to a cysteine thiol in a protein of a glutathione group), and tyrosine nitration (i.e., attachment to tyrosine residues of proteins of nitrate groups).

The term “produced recombinantly” as used herein in reference to a component (e.g., a protein, a lipid) refers to a component that is produced in a cell of a different species or type as compared to the species or type of cell that produces the component in nature (e.g., a recombinant host cell), or that is produced in a cell at a level at which it is not produced in nature, or that is produced using a recombinant polynucleotide.

The term “recombinant host cell” as used herein refers to a host cell that comprises a recombinant polynucleotide. Thus, for example, a recombinant host cell may produce a polynucleotide or polypeptide not found in the native (non-recombinant) form of the host cell, or a recombinant host cell may produce a polynucleotide or polypeptide at a level that is different from that in the native (non-recombinant) form of the host cell. It should be understood that such term is intended to refer not only to the particular subject cell but also to the progeny of such a cell. Because certain modifications may occur in succeeding generations due to either mutation or environmental influences, such progeny may not be identical to the subject cell, but are still included within the scope of the term “recombinant host cell” as used herein. A recombinant host cell can be an isolated cell or cell line grown in culture or can be a cell which resides in a living tissue or organism.

The term “recombinant polynucleotide” as used herein refers to a polynucleotide that is removed from its naturally occurring environment, or a polynucleotide that is not associated with all or a portion of a polynucleotide abutting or proximal to the polynucleotide when it is found in nature, or a polynucleotide that is operatively linked to a polynucleotide that it is not linked to in nature, or a polynucleotide that does not occur in nature, or a polynucleotide that contains a modification that is not found in that polynucleotide in nature (e.g., insertion, deletion, or point mutation introduced artificially, e.g., by human intervention), or a polynucleotide that is integrated into a chromosome at a heterologous site. The term can be used, e.g., to describe cloned DNA isolates, or a polynucleotide comprising a chemically synthesized nucleotide analog. A polynucleotide is also considered “recombinant” if it contains a genetic modification that does not naturally occur. For instance, an endogenous polynucleotide is considered a “recombinant polynucleotide” if it contains an insertion, deletion, or substitution of one or more nucleotides that is introduced artificially (e.g., by human intervention). Such modification can introduce into the polynucleotide a point mutation, substitution mutation, deletion mutation, insertion mutation, missense mutation, frameshift mutation, duplication mutation, amplification mutation, translocation mutation, or inversion mutation. The term includes a polynucleotide in a host cell's chromosome, as well as a polynucleotide that is not in a host cell's chromosome (e.g., a polynucleotide that is comprised in an episome). A recombinant polynucleotide in a host cell or organism may replicate using the in vivo cellular machinery of the host cell; however, such recombinant polynucleotide, although subsequently replicated intracellularly, is still considered recombinant for purposes of this invention.

The term “regulatory element” as used herein refers a polynucleotide sequence that mediates, modulates, or regulates expression (e.g., transcription, post-transcriptional events, translation) of a polynucleotide to which the regulatory element is operably linked. Non-limiting examples of regulatory elements include promoter sequences, termination sequences, transcriptional start sequences, translational start sequences, translation stop sequences, enhancer sequences, activator sequences, response elements, protein recognition sites, inducible elements, protein binding sequences, 5′ and 3′ untranslated regions, introns, operators (i.e., sequences of nucleic acids adjacent to a promoter that comprise a protein-binding domain where a repressor protein can bind and reduce or eliminate activity of the promoter), efficient RNA processing signals (e.g., splicing signals, polyadenylation signals), sequences that stabilize cytoplasmic mRNA, sequences that enhance translation efficiency (e.g., ribosome binding sites [e.g., Shine-Dalgarno sequences]), sequences that enhance protein stability, and sequences that enhance protein secretion.

The term “recombinant milk lipid” as used herein refers to a milk lipid that is produced recombinantly.

The term “saturated” as used herein refers to not comprising any carbon-carbon double or triple bond.

The term “similar” us used herein refers to being within about +/−20% with regard to a specified attribute. The term includes being within about +/−20%, about +/−17%, about +/−15%, about +/−12%, about +/−10%, about +/−9%, about +/−8%, about +/−7%, about +/−6%, about +/−5%, about +/−4%, about +/−3%, about +/−2%, or about +/−1% with regard to the specified attribute.

The terms “sn1 position”, “sn2 position”, and “sn3 position” as used herein refer to the stereospecific positions of the first, second, and third carbon, respectively, in a glycerol molecule that can form an ester bond with a fatty acid. Specifically, the carbon atom that appears on top in a Fischer projection of the glycerol molecule that shows a vertical carbon chain with the hydroxyl group at the carbon atom at position 2 to the left is designated as sn1, the carbon atom in the middle as sn2, and the carbon atom that appears on bottom as sn3.

The term “structured” as used herein in reference to a lipid refers to a lipid in which fatty acids are exchanged with other fatty acids (i.e., inter-esterified).

The term “texture” as used herein refers to mechanical characteristics of a food product that are correlated with sensory perceptions of the food product.

The term “two or more” as used herein refer to two, three, four, five, six, seven, eight, nine, ten, or more; less than 5, less than 10, less than 15, less than 20, less than 30, less than 40, less than 50, less than 60, less than 70, less than 80, less than 90, less than 100, or less than 500; at least 2, at least 5, at least 10, at least 25, at least 50, or at least 100; or all of the elements subsequently listed.

The term “unsaturated fatty acid” as used herein refers to a fatty acid that comprises at least one carbon-to-carbon double or triple bond.

The term “yeast” as used herein refers to organisms of the order Saccharomycetales. Vegetative growth of yeast is by budding/blebbing of a unicellular thallus, and carbon catabolism may be fermentative. The term “yeast cell” as used herein refers to a cell that is obtained from a yeast.

Recitation of ranges of values herein are merely intended to serve as a shorthand method of referring individually to each separate value (fractional or integral) falling within the range inclusive of the recited minimum and maximum value, unless otherwise indicated herein, and each separate value is incorporated into the specification as if it were individually recited herein. Also, it should be understood that any numerical range recited herein is intended to include all sub-ranges subsumed therein. For example, a range of “4 to 54” is intended to include all sub-ranges between (and including) the recited minimum value of 4 and the recited maximum value of 54, that is, having a minimum value equal to or greater than 4, and a maximum value of less than or equal to 54.

As an example, a range of “4 to 54” includes at least any of the following ranges: 4 to 54, 52, 50, 48, 46, 44, 42, 40, 38, 36, 34, 32, 30, 28, 26, 24, 22, 20, 18, 16, 14, 12, 10, 8, or 6; 6 to 54, 52, 50, 48, 46, 44, 42, 40, 38, 36, 34, 32, 30, 28, 26, 24, 22, 20, 18, 16, 14, 12, 10, or 8; 8 to 54, 52, 50, 48, 46, 44, 42, 40, 38, 36, 34, 32, 30, 28, 26, 24, 22, 20, 18, 16, 14, 12, or 10; 10 to 54, 52, 50, 48, 46, 44, 42, 40, 38, 36, 34, 32, 30, 28, 26, 24, 22, 20, 18, 16, 14, or 12; 12 to 54, 52, 50, 48, 46, 44, 42, 40, 38, 36, 34, 32, 30, 28, 26, 24, 22, 20, 18, 16, or 14; 14 to 54, 52, 50, 48, 46, 44, 42, 40, 38, 36, 34, 32, 30, 28, 26, 24, 22, 20, 18, or 16; 16 to 54, 52, 50, 48, 46, 44, 42, 40, 38, 36, 34, 32, 30, 28, 26, 24, 22, 20, or 18; 18 to 54, 52, 50, 48, 46, 44, 42, 40, 38, 36, 34, 32, 30, 28, 26, 24, 22, or 20; 20 to 54, 52, 50, 48, 46, 44, 42, 40, 38, 36, 34, 32, 30, 28, 26, 24, or 22; 22 to 54, 52, 50, 48, 46, 44, 42, 40, 38, 36, 34, 32, 30, 28, 26, or 24; 24 to 54, 52, 50, 48, 46, 44, 42, 40, 38, 36, 34, 32, 30, 28, or 26; 26 to 54, 52, 50, 48, 46, 44, 42, 40, 38, 36, 34, 32, 30, or 28; 28 to 54, 52, 50, 48, 46, 44, 42, 40, 38, 36, 34, 32, or 30; 30 to 54, 52, 50, 48, 46, 44, 42, 40, 38, 36, 34, or 32; 32 to 54, 52, 50, 48, 46, 44, 42, 40, 38, 36, or 34; 34 to 54, 52, 50, 48, 46, 44, 42, 40, 38, or 36; 36 to 54, 52, 50, 48, 46, 44, 42, 40, or 38; 38 to 54, 52, 50, 48, 46, 44, 42, or 40; 40 to 54, 52, 50, 48, 46, 44, or 42; 42 to 54, 52, 50, 48, 46, or 44; 44 to 54, 52, 50, 48, or 46; 46 to 54, 52, 50, or 48; 48 to 54, 52, or 50; 50 to 54, or 52; and 52 to 54.

As a further example, a range of “4 to 24” includes at least any of the following ranges: 4 to 24, 22, 20, 18, 16, 14, 12, 10, 8, or 6; 6 to 24, 22, 20, 18, 16, 14, 12, 10, or 8; 8 to 24, 22, 20, 18, 16, 14, 12, or 10; 10 to 24, 22, 20, 18, 16, 14, or 12; 12 to 24, 22, 20, 18, 16, or 14; 14 to 24, 22, 20, 18, or 16; 16 to 24, 22, 20, or 18; 18 to 24, 22, or 20; 20 to 24, or 22; and 22 to 24.

As a further example, a range of “4 to 10” includes at least any of the following ranges: 4 to 10, 8, or 6; 6 to 10, or 8; and 8 to 10.

As a further example, a range of “8 to 16” includes at least any of the following ranges: 8 to 16, 14, 12, or 10; 10 to 16, 14, or 12; 12 to 16, or 14; and 14 to 16.

As a further example, a range of “16 to 54” includes at least any of the following ranges: 16 to 54, 52, 50, 48, 46, 44, 42, 40, 38, 36, 34, 32, 30, 28, 26, 24, 22, 20, or 18; 18 to 54, 52, 50, 48, 46, 44, 42, 40, 38, 36, 34, 32, 30, 28, 26, 24, 22, or 20; 20 to 54, 52, 50, 48, 46, 44, 42, 40, 38, 36, 34, 32, 30, 28, 26, 24, or 22; 22 to 54, 52, 50, 48, 46, 44, 42, 40, 38, 36, 34, 32, 30, 28, 26, or 24; 24 to 54, 52, 50, 48, 46, 44, 42, 40, 38, 36, 34, 32, 30, 28, or 26; 26 to 54, 52, 50, 48, 46, 44, 42, 40, 38, 36, 34, 32, 30, or 28; 28 to 54, 52, 50, 48, 46, 44, 42, 40, 38, 36, 34, 32, or 30; 30 to 54, 52, 50, 48, 46, 44, 42, 40, 38, 36, 34, or 32; 32 to 54, 52, 50, 48, 46, 44, 42, 40, 38, 36, or 34; 34 to 54, 52, 50, 48, 46, 44, 42, 40, 38, or 36; 36 to 54, 52, 50, 48, 46, 44, 42, 40, or 38; 38 to 54, 52, 50, 48, 46, 44, 42, or 40; 40 to 54, 52, 50, 48, 46, 44, or 42; 42 to 54, 52, 50, 48, 46, or 44; 44 to 54, 52, 50, 48, or 46; 46 to 54, 52, 50, or 48; 48 to 54, 52, or 50; 50 to 54, or 52; and 52 to 54.

Where ranges and numbers are used these can be approximate to include statistical ranges or measurement errors or variation (for example, measurements could be plus or minus 10%).

### Milk Lipid Component

In one aspect, provided herein is a milk lipid component. The term “milk lipid component” as used herein refers to a component comprising a subset of milk lipids (i.e., just some but not all lipids present in a mammal-produced milk or milk fat).

The invention is based on the discovery of methods and reagents for producing structured and/or recombinant milk lipids that enable production of a milk lipid component that has a fatty acid profile and/or glycerolipid profile that is not naturally available from non-animal sources, and that can impart a desirable attribute on a composition. The invention is further based on the discovery that a milk lipid component can impart a desirable attribute on a composition.

The invention is useful as it can in some embodiments provide advantages in production of milk lipids, including but not limited to: independence from the productivity of animal farming; independence from market uncertainties due to outbreaks of disease among livestock; and no negative impact on animal welfare (e.g., no animal confinement, force feeding, or hormone treatment).

Additional potential advantages include a more limited negative impact on the environment (i.e., smaller natural resources requirements [e.g., less water, land, energy used], lower carbon dioxide production); mitigation of supply chain and production risk (e.g., use of non-animal lipids obtained from a greater variety of natural sources providing supply chain variations and increased flexibility in production methods); lower production costs; being devoid of unhealthy components obtained from animals (e.g., trans fatty acids, cholesterol, microbial contaminants [e.g., Salmonella]) and/or plant components (e.g., plant contaminants); having similar or superior nutrient content (e.g., favorable lipid profiles [e.g., higher level of mono- and polyunsaturated fatty acids], higher content of lipid-soluble vitamins [e.g., vitamin E]); having a desirable flavor profile (e.g., a milk- or dairy-like flavor profile); being specifically engineered to have desirable or novel attributes; and/or providing improved functionality (e.g., better butter spreadability, better cream whip ability) and use versatility.

The milk lipid component of any of the above can consist of a single milk lipid, or of two or more distinct milk lipids.

The milk lipid component of any of the above can consist of or comprise one or more mTAGs, one or more mDAGs, one or more mMAGs, one or more mPLs, one or more milk sterols, one or more mFFAs, or any combination thereof.

The milk lipid component of any of the above can consist of or comprise one or more bovine milk lipids (i.e., milk lipids found in a milk or milk fat obtained from cow), one or more sheep milk lipids (i.e., milk lipids found in a milk or milk fat obtained from sheep), one or more goat milk lipids (i.e., milk lipids found in a milk or milk fat obtained from goat), one or more human milk lipids (i.e., milk lipids found in a milk or milk fat obtained from human), or any combination thereof.

The milk lipid component of any of the above can be essentially free of, or comprise a lower or higher concentration of, one or more milk lipids, compared to a mammal-produced milk or milk fat.

The milk lipid component of any of the above can be essentially free of, or comprise a lower or higher concentration of, one or more mono-unsaturated fatty acids present in a mammal-produced milk or milk fat, compared to a mammal-produced milk fat.

The milk lipid component of any of the above can be essentially free of, or comprise a lower or higher concentration of, one or more saturated fatty acids present in a mammal-produced milk or milk fat, compared to a mammal-produced milk or milk fat.

The milk lipid component of any of the above can be essentially free of one or more fatty acids having a carbon atom number of greater than 50, greater than 48, greater than 46, greater than 44, greater than 42, greater than 40, greater than 38, greater than 36, greater than 34, greater than 32, greater than 30, greater than 28, greater than 26, greater than 24, greater than 22, greater than 20, and/or greater than 18.

The milk lipid component of any of the above can be essentially free of, or comprise a lower or higher concentration of, cholesterol, compared to a mammal-produced milk or milk fat. The milk lipid component of any of the above can comprise less than 2%, less than 1.5%, less than 1%, less than 0.5%, less than 0.1%, less than 0.05%, or less than 0.01% by mass of cholesterol.

The milk lipid component of any of the above can comprise one or more milk lipids at relative ratios found in a mammal-produced milk or milk fat; or at relative ratios not found in a mammal-produced milk or milk fat.

The milk lipid component of any of the above can have a fatty acid profile that is identical to the fatty acid profile of a mammal-produced milk or milk fat, or a fatty acid profile that is similar to the fatty acid profile of a mammal-produced milk or milk fat, or a fatty acid profile that is different from the fatty acid profile of a mammal-produced milk or milk fat.

The milk lipid component of any of the above can comprise the following fatty acids in the following amounts: between 0% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, 4%, or 2%; between 2% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, or 4%; between 4% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, or 6%; between 6% and 50%, 48%, 46%, 44%, 42%, 40%, 38%, 36%, 34%, 32%, 30%, 28%, 26%, 24%, 22%, 20%, 18%, 16%, 14%, 12%, 10%, or 8%; between 8% and 50%, 48%, 46%, 44%, 42%, 40%, 38%, 36%, 34%, 32%, 30%, 28%, 26%, 24%, 22%, 20%, 18%, 16%, 14%, 12%, or 10%; between 10% and 50%, 48%, 46%, 44%, 42%, 40%, 38%, 36%, 34%, 32%, 30%, 28%, 26%, 24%, 22%, 20%, 18%, 16%, 14%, or 12%; between 12% and 50%, 48%, 46%, 44%, 42%, 40%, 38%, 36%, 34%, 32%, 30%, 28%, 26%, 24%, 22%, 20%, 18%, 16%, or 14%; between 14% and 50%, 48%, 46%, 44%, 42%, 40%, 38%, 36%, 34%, 32%, 30%, 28%, 26%, 24%, 22%, 20%, 18%, or 16%; between 16% and 50%, 48%, 46%, 44%, 42%, 40%, 38%, 36%, 34%, 32%, 30%, 28%, 26%, 24%, 22%, 20%, or 18%; between 18% and 50%, 48%, 46%, 44%, 42%, 40%, 38%, 36%, 34%, 32%, 30%, 28%, 26%, 24%, 22%, or 20%; between 20% and 50%, 48%, 46%, 44%, 42%, 40%, 38%, 36%, 34%, 32%, 30%, 28%, 26%, 24%, or 22%; between 22% and 50%, 48%, 46%, 44%, 42%, 40%, 38%, 36%, 34%, 32%, 30%, 28%, 26%, or 24%; between 24% and 50%, 48%, 46%, 44%, 42%, 40%, 38%, 36%, 34%, 32%, 30%, 28%, 26%; between 26% and 50%, 48%, 46%, 44%, 42%, 40%, 38%, 36%, 34%, 32%, 30%, or 28%; between 28% and 50%, 48%, 46%, 44%, 42%, 40%, 38%, 36%, 34%, 32%, or 30%; between 30% and 50%, 48%, 46%, 44%, 42%, 40%, 38%, 36%, 34%, or 32%; between 32% and 50%, 48%, 46%, 44%, 42%, 40%, 38%, 36%, or 34%; between 34% and 50%, 48%, 46%, 44%, 42%, 40%, 38%, or 36%; between 36% and 50%, 48%, 46%, 44%, 42%, 40%, or 38%; between 38% and 50%, 48%, 46%, 44%, 42%, or 40%; between 40% and 50%, 48%, 46%, 44%, or 42%; between 42% and 50%, 48%, 46%, or 44%; between 44% and 50%, 48%, or 46%; between 46% and 50%, or 48%; or between 48% and 50% by mass of butyric acid (C4:0); between 0% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, 4%, or 2%; between 2% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, or 4%; between 4% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, or 6%; between 6% and 50%, 48%, 46%, 44%, 42%, 40%, 38%, 36%, 34%, 32%, 30%, 28%, 26%, 24%, 22%, 20%, 18%, 16%, 14%, 12%, 10%, or 8%; between 8% and 50%, 48%, 46%, 44%, 42%, 40%, 38%, 36%, 34%, 32%, 30%, 28%, 26%, 24%, 22%, 20%, 18%, 16%, 14%, 12%, or 10%; between 10% and 50%, 48%, 46%, 44%, 42%, 40%, 38%, 36%, 34%, 32%, 30%, 28%, 26%, 24%, 22%, 20%, 18%, 16%, 14, or 12%; between 12% and 50%, 48%, 46%, 44%, 42%, 40%, 38%, 36%, 34%, 32%, 30%, 28%, 26%, 24%, 22%, 20%, 18%, 16%, or 14%; between 14% and 50%, 48%, 46%, 44%, 42%, 40%, 38%, 36%, 34%, 32%, 30%, 28%, 26%, 24%, 22%, 20%, 18%, or 16%; between 16% and 50%, 48%, 46%, 44%, 42%, 40%, 38%, 36%, 34%, 32%, 30%, 28%, 26%, 24%, 22%, 20%, or 18%; between 18% and 50%, 48%, 46%, 44%, 42%, 40%, 38%, 36%, 34%, 32%, 30%, 28%, 26%, 24%, 22%, or 20%; between 20% and 50%, 48%, 46%, 44%, 42%, 40%, 38%, 36%, 34%, 32%, 30%, 28%, 26%, 24%, or 22%; between 22% and 50%, 48%, 46%, 44%, 42%, 40%, 38%, 36%, 34%, 32%, 30%, 28%, 26%, or 24%; between 24% and 50%, 48%, 46%, 44%, 42%, 40%, 38%, 36%, 34%, 32%, 30%, 28%, 26%; between 26% and 50%, 48%, 46%, 44%, 42%, 40%, 38%, 36%, 34%, 32%, 30%, or 28%; between 28% and 50%, 48%, 46%, 44%, 42%, 40%, 38%, 36%, 34%, 32%, or 30%; between 30% and 50%, 48%, 46%, 44%, 42%, 40%, 38%, 36%, 34%, or 32%; between 32% and 50%, 48%, 46%, 44%, 42%, 40%, 38%, 36%, or 34%; between 34% and 50%, 48%, 46%, 44%, 42%, 40%, 38%, or 36%; between 36% and 50%, 48%, 46%, 44%, 42%, 40%, or 38%; between 38% and 50%, 48%, 46%, 44%, 42%, or 40%; between 40% and 50%, 48%, 46%, 44%, or 42%; between 42% and 50%, 48%, 46%, or 44%; between 44% and 50%, 48%, or 46%; between 46% and 50%, or 48%; or between 48% and 50% by mass of caproic acid (C6:0); between 0% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, 4%, or 2%; between 2% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, or 4%; between 4% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, or 6%; between 6% and 50%, 48%, 46%, 44%, 42%, 40%, 38%, 36%, 34%, 32%, 30%, 28%, 26%, 24%, 22%, 20%, 18%, 16%, 14%, 12%, 10%, or 8%; between 8% and 50%, 48%, 46%, 44%, 42%, 40%, 38%, 36%, 34%, 32%, 30%, 28%, 26%, 24%, 22%, 20%, 18%, 16%, 14%, 12%, or 10%; between 10% and 50%, 48%, 46%, 44%, 42%, 40%, 38%, 36%, 34%, 32%, 30%, 28%, 26%, 24%, 22%, 20%, 18%, 16%, 14, or 12%; between 12% and 50%, 48%, 46%, 44%, 42%, 40%, 38%, 36%, 34%, 32%, 30%, 28%, 26%, 24%, 22%, 20%, 18%, 16%, or 14%; between 14% and 50%, 48%, 46%, 44%, 42%, 40%, 38%, 36%, 34%, 32%, 30%, 28%, 26%, 24%, 22%, 20%, 18%, or 16%; between 16% and 50%, 48%, 46%, 44%, 42%, 40%, 38%, 36%, 34%, 32%, 30%, 28%, 26%, 24%, 22%, 20%, or 18%; between 18% and 50%, 48%, 46%, 44%, 42%, 40%, 38%, 36%, 34%, 32%, 30%, 28%, 26%, 24%, 22%, or 20%; between 20% and 50%, 48%, 46%, 44%, 42%, 40%, 38%, 36%, 34%, 32%, 30%, 28%, 26%, 24%, or 22%; between 22% and %, 48%, 46%, 44%, 42%, 40%, 38%, 36%, 340% 328%, 30%, 28%, 26%, or 24%; between 24% and 504%, 48%, 46%, 44% 42%, 40%, 38%, 36%, 34% 32%, 30%, 28%, 26%; between 26% and 50%, 48%, 46%, 44%, 42%, 40%, 38%, 36%, 34%, 32%, 30%, or 28%; between 28% and 50%, 48%, 46%, 44%, 42%, 40%, 38%, 36%, 34%, 32%, or 30%; between 30% and 50%, 48%, 46%, 44%, 42%, 40%, 38%, 36%, 34%, or 32%; between 32% and 50%, 48%, 46%, 44%, 42%, 40%, 38%, 36%, or 34%; between 34% and 50%, 48%, 46%, 44%, 42%, 40%, 38%, or 36%; between 36% and 50%, 48%, 46%, 44%, 42%, 40%, or 38%; between 38% and 50%, 48%, 46%, 44%, 42%, or 40%; between 40% and 50%, 48%, 46%, 44%, or 42%; between 42% and 50%, 48%, 46%, or 44%; between 44% and 50%, 48%, or 46%; between 46% and 50%, or 48%; or between 48% and 50% by mass of caprylic acid (C8:0); between 0% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, 4%, or 2%; between 2% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, or 4%; between 4% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, or 6%; between 6% and 50%, 48%, 46%, 44%, 42%, 40%, 38%, 36%, 34%, 32%, 30%, 28%, 26%, 24%, 22%, 20%, 18%, 16%, 14%, 12%, 10%, or 8; between 8% and 50%, 48%, 46%, 44%, 42%, 40%, 38%, 36%, 34%, 32%, 30%, 28%, 26%, 24%, 22%, 20%, 18%, 16%, 14%, 12, or 10%; between 10% and 50%, 48%, 46%, 44%, 42%, 40%, 38%, 36%, 34%, 32%, 30%, 28%, 26%, 24%, 22%, 20%, 18%, 16%, 14, or 12%; between 12% and 50%, 48%, 46%, 44%, 42%, 40%, 38%, 36%, 34%, 32%, 30%, 28%, 26%, 24%, 22%, 20%, 18%, 16%, or 14%; between 14% and 50%, 48%, 46%, 44%, 42%, 40%, 38%, 36%, 34%, 32%, 30%, 28%, 26%, 24%, 22%, 20%, 18%, or 16%; between 16% and 50%, 48%, 46%, 44%, 42%, 40%, 38%, 36%, 34%, 32%, 30%, 28%, 26%, 24%, 22%, 20%, or 18%; between 18% and 50%, 48%, 46%, 44%, 42%, 40%, 38%, 36%, 34%, 32%, 30%, 28%, 26%, 24%, 22%, or 20%; between 20% and 50%, 48%, 46%, 44%, 42%, 40%, 38%, 36%, 34%, 32%, 30%, 28%, 26%, 24%, or 22%; between 22% and 50%, 48%, 46%, 44%, 42%, 40%, 38%, 36%, 34%, 32%, 30%, 28%, 26%, or 24%; between 24% and 50%, 48%, 46%, 44%, 42%, 40%, 38%, 36%, 34%, 32%, 30%, 28%, 26%; between 26% and 50%, 48%, 46%, 44%, 42%, 40%, 38%, 36%, 34%, 32%, 30%, or 28%; between 28% and 50%, 48%, 46%, 44%, 42%, 40%, 38%, 36%, 34%, 32%, or 30%; between 30% and 50%, 48%, 46%, 44%, 42%, 40%, 38%, 36%, 34%, or 32%; between 32% and 50%, 48%, 46%, 44%, 42%, 40%, 38%, 36%, or 34%; between 34% and 50%, 48%, 46%, 44%, 42%, 40%, 38%, or 36%; between 36% and 50%, 48%, 46%, 44%, 42%, 40%, or 38%; between 38% and 50%, 48%, 46%, 44%, 42%, or 40%; between 40% and 50%, 48%, 46%, 44%, or 42%; between 42% and 50%, 48%, 46%, or 44%; between 44% and 50%, 48%, or 46%; between 46% and 50%, or 48%; or between 48% and 50% by mass of capric acid (C10:0); between 0% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, 4%, or 2%; between 2% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, or 4%; between 4% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, or 6%; between 6% and 50%, 48%, 46%, 44%, 42%, 40%, 38%, 36%, 34%, 32%, 30%, 28%, 26%, 24%, 22%, 20%, 18%, 16%, 14%, 12%, 10%, or 8%; between 8% and 50%, 48%, 46%, 44%, 42%, 40%, 38%, 36%, 34%, 32%, 30%, 28%, 26%, 24%, 22%, 20%, 18%, 16%, 14%, 12%, or 10%; between 10% and 50%, 48%, 46%, 44%, 42%, 40%, 38%, 36%, 34%, 32%, 30%, 28%, 26%, 24%, 22%, 20%, 18%, 16%, 14%, or 12%; between 12% and 50%, 48%, 46%, 44%, 42%, 40%, 38%, 36%, 34%, 32%, 30%, 28%, 26%, 24%, 22%, 20%, 18%, 16%, or 14%; between 14% and 50%, 48%, 46%, 44%, 42%, 40%, 38%, 36%, 34%, 32%, 30%, 28%, 26%, 24%, 22%, 20%, 18%, or 16%; between 16% and 50%, 48%, 46%, 44%, 42%, 40%, 38%, 36%, 34%, 32%, 30%, 28%, 26%, 24%, 22%, 20%, or 18%; between 18% and 50%, 48%, 46%, 44%, 42%, 40%, 38%, 36%, 34%, 32%, 30%, 28%, 26%, 24%, 22%, or 20%; between 20% and 50%, 48%, 46%, 44%, 42%, 40%, 38%, 36%, 34%, 32%, 30%, 28%, 26%, 24%, or 22%; between 22% and 50%, 48%, 46%, 44%, 42%, 40%, 38%, 36%, 34%, 32%, 30%, 28%, 26%, or 24%; between 24% and 50%, 48%, 46%, 44%, 42%, 40%, 38%, 36%, 34%, 32%, 30%, 28%, 26%; between 26% and 50%, 48%, 46%, 44%, 42%, 40%, 38%, 36%, 34%, 32%, 30%, or 28%; between 28% and 50%, 48%, 46%, 44%, 42%, 40%, 38%, 36%, 34%, 32%, or 30%; between 30% and 50%, 48%, 46%, 44%, 42%, 40%, 38%, 36%, 34%, or 32%; between 32% and 50%, 48%, 46%, 44%, 42%, 40%, 38%, 36%, or 34%; between 34% and 50%, 48%, 46%, 44%, 42%, 40%, 38%, or 36%; between 36% and 50%, 48%, 46%, 44%, 42%, 40%, or 38%; between 38% and 50%, 48%, 46%, 44%, 42%, or 40%; between 40% and 50%, 48%, 46%, 44%, or 42%; between 42% and 50%, 48%, 46%, or 44%; between 44% and 50%, 48%, or 46%; between 46% and 50%, or 48%; or between 48% and 50% by mass of lauric acid (C12:0); between 0% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, 4%, or 2%; between 2% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, or 4%; between 4% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, or 6%; between 6% and 50%, 48%, 46%, 44%, 42%, 40%, 38%, 36%, 34%, 32%, 30%, 28%, 26%, 24%, 22%, 20%, 18%, 16%, 14%, 12%, 10%, or 8%; between 8% and 50%, 48%, 46%, 44%, 42%, 40%, 38%, 36%, 34%, 32%, 30%, 28%, 26%, 24%, 22%, 20%, 18%, 16%, 14%, 12%, or 10%; between 10% and 50%, 48%, 46%, 44%, 42%, 40%, 38%, 36%, 34%, 32%, 30%, 28%, 26%, 24%, 22%, 20%, 18%, 16%, 14%, or 12%; between 12% and 50%, 48%, 46%, 44%, 42%, 40%, 38%, 36%, 34%, 32%, 30%, 28%, 26%, 24%, 22%, 20%, 18%, 16%, or 14%; between 14% and 50%, 48%, 46%, 44%, 42%, 40%, 38%, 36%, 34%, 32%, 30%, 28%, 26%, 24%, 22%, 20%, 18%, or 16%; between 16% and 50%, 48%, 46%, 44%, 42%, 40%, 38%, 36%, 34%, 32%, 30%, 28%, 26%, 24%, 22%, 20%, or 18%; between 18% and 50%, 48%, 46%, 44%, 42%, 40%, 38%, 36%, 34%, 32%, 30%, 28%, 26%, 24%, 22%, or 20%; between 20% and 50%, 48%, 46%, 44%, 42%, 40%, 38%, 36%, 34%, 32%, 30%, 28%, 26%, 24%, or 22%; between 22% and 50%, 48%, 46%, 44%, 42%, 40%, 38%, 36%, 34%, 32%, 30%, 28%, 26%, or 24%; between 24% and 50%, 48%, 46%, 44%, 42%, 40%, 38%, 36%, 34%, 32%, 30%, 28%, 26%; between 26% and 50%, 48%, 46%, 44%, 42%, 40%, 38%, 36%, 34%, 32%, 30%, or 28%; between 28% and 50%, 48%, 46%, 44%, 42%, 40%, 38%, 36%, 34%, 32%, or 30%; between 30% and 50%, 48%, 46%, 44%, 42%, 40%, 38%, 36%, 34%, or 32%; between 32% and 50%, 48%, 46%, 44%, 42%, 40%, 38%, 36%, or 34%; between 34% and 50%, 48%, 46%, 44%, 42%, 40%, 38%, or 36%; between 36% and 50%, 48%, 46%, 44%, 42%, 40%, or 38%; between 38% and 50%, 48%, 46%, 44%, 42%, or 40%; between 40% and 50%, 48%, 46%, 44%, or 42%; between 42% and 50%, 48%, 46%, or 44%; between 44% and 50%, 48%, or 46%; between 46% and 50%, or 48%; or between 48% and 50% by mass of myristic acid (C14:0); between 0% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, 4%, or 2%; between 2% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, or 4%; between 4% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, or 6%; between 6% and 50%, 48%, 46%, 44%, 42%, 40%, 38%, 36%, 34%, 32%, 30%, 28%, 26%, 24%, 22%, 20%, 18%, 16%, 14%, 12%, 10%, or 8%; between 8% and 50%, 48%, 46%, 44%, 42%, 40%, 38%, 36%, 34%, 32%, 30%, 28%, 26%, 24%, 22%, 20%, 18%, 16%, 14%, 12%, or 10%; between 10% and 50%, 48%, 46%, 44%, 42%, 40%, 38%, 36%, 34%, 32%, 30%, 28%, 26%, 24%, 22%, 20%, 18%, 16%, 14%, or 12%; between 12% and 50%, 48%, 46%, 44%, 42%, 40%, 38%, 36%, 34%, 32%, 30%, 28%, 26%, 24%, 22%, 20%, 18%, 16%, or 14%; between 14% and 50%, 48%, 46%, 44%, 42%, 40%, 38%, 36%, 34%, 32%, 30%, 28%, 26%, 24%, 22%, 20%, 18%, or 16%; between 16% and 50%, 48%, 46%, 44%, 42%, 40%, 38%, 36%, 34%, 32%, 30%, 28%, 26%, 24%, 22%, 20%, or 18%; between 18% and 50%, 48%, 46%, 44%, 42%, 40%, 38%, 36%, 34%, 32%, 30%, 28%, 26%, 24%, 22%, or 20%; between 20% and 50%, 48%, 46%, 44%, 42%, 40%, 38%, 36%, 34%, 32%, 30%, 28%, 26%, 24%, or 22%; between 22% and 50%, 48%, 46%, 44%, 42%, 40%, 38%, 36%, 34%, 32%, 30%, 28%, 26%, or 24%; between 24% and 50%, 48%, 46%, 44%, 42%, 40%, 38%, 36%, 34%, 32%, 30%, 28%, 26%; between 26% and 50%, 48%, 46%, 44%, 42%, 40%, 38%, 36%, 34%, 32%, 30%, or 28%; between 28% and 50%, 48%, 46%, 44%, 42%, 40%, 38%, 36%, 34%, 32%, or 30%; between 30% and 50%, 48%, 46%, 44%, 42%, 40%, 38%, 36%, 34%, or 32%; between 32% and 50%, 48%, 46%, 44%, 42%, 40%, 38%, 36%, or 34%; between 34% and 50%, 48%, 46%, 44%, 42%, 40%, 38%, or 36%; between 36% and 50%, 48%, 46%, 44%, 42%, 40%, or 38%; between 38% and 50%, 48%, 46%, 44%, 42%, or 40%; between 40% and 50%, 48%, 46%, 44%, or 42%; between 42% and 50%, 48%, 46%, or 44%; between 44% and 50%, 48%, or 46%; between 46% and 50%, or 48%; or between 48% and 50% by mass of pentadecanoic acid (C15:0); between 0% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, 4%, or 2%; between 2% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, or 4%; between 4% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, or 6%; between 6% and 50%, 48%, 46%, 44%, 42%, 40%, 38%, 36%, 34%, 32%, 30%, 28%, 26%, 24%, 22%, 20%, 18%, 16%, 14%, 12%, 10%, or 8%; between 8% and 50%, 48%, 46%, 44%, 42%, 40%, 38%, 36%, 34%, 32%, 30%, 28%, 26%, 24%, 22%, 20%, 18%, 16%, 14%, 12%, or 10%; between 10% and 50%, 48%, 46%, 44%, 42%, 40%, 38%, 36%, 34%, 32%, 30%, 28%, 26%, 24%, 22%, 20%, 18%, 16%, 14%, or 12%; between 12% and 50%, 48%, 46%, 44%, 42%, 40%, 38%, 36%, 34%, 32%, 30%, 28%, 26%, 24%, 22%, 20%, 18%, 16%, or 14%; between 14% and 50%, 48%, 46%, 44%, 42%, 40%, 38%, 36%, 34%, 32%, 30%, 28%, 26%, 24%, 22%, 20%, 18%, or 16%; between 16% and 50%, 48%, 46%, 44%, 42%, 40%, 38%, 36%, 34%, 32%, 30%, 28%, 26%, 24%, 22%, 20%, or 18%; between 18% and 50%, 48%, 46%, 44%, 42%, 40%, 38%, 36%, 34%, 32%, 30%, 28%, 26%, 24%, 22%, or 20%; between 20% and 50%, 48%, 46%, 44%, 42%, 40%, 38%, 36%, 34%, 32%, 30%, 28%, 26%, 24%, or 22%; between 22% and 50%, 48%, 46%, 44%, 42%, 40%, 38%, 36%, 34%, 32%, 30%, 28%, 26%, or 24%; between 24% and 50%, 48%, 46%, 44%, 42%, 40%, 38%, 36%, 34%, 32%, 30%, 28%, 26%; between 26% and 50%, 48%, 46%, 44%, 42%, 40%, 38%, 36%, 34%, 32%, 30%, or 28%; between 28% and 50%, 48%, 46%, 44%, 42%, 40%, 38%, 36%, 34%, 32%, or 30%; between 30% and 50%, 48%, 46%, 44%, 42%, 40%, 38%, 36%, 34%, or 32%; between 32% and 50%, 48%, 46%, 44%, 42%, 40%, 38%, 36%, or 34%; between 34% and 50%, 48%, 46%, 44%, 42%, 40%, 38%, or 36%; between 36% and 50%, 48%, 46%, 44%, 42%, 40%, or 38%; between 38% and 50%, 48%, 46%, 44%, 42%, or 40%; between 40% and 50%, 48%, 46%, 44%, or 42%; between 42% and 50%, 48%, 46%, or 44%; between 44% and 50%, 48%, or 46%; between 46% and 50%, or 48%; or between 48% and 50% by mass of palmitic acid (C16:0); between 0% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, 4%, or 2%; between 2% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, or 4%; between 4% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, or 6%; between 6% and 50%, 48%, 46%, 44%, 42%, 40%, 38%, 36%, 34%, 32%, 30%, 28%, 26%, 24%, 22%, 20%, 18%, 16%, 14%, 12%, 10%, or 8%; between 8% and 50%, 48%, 46%, 44%, 42%, 40%, 38%, 36%, 34%, 32%, 30%, 28%, 26%, 24%, 22%, 20%, 18%, 16%, 14%, 12%, or 10%; between 10% and 50%, 48%, 46%, 44%, 42%, 40%, 38%, 36%, 34%, 32%, 30%, 28%, 26%, 24%, 22%, 20%, 18%, 16%, 14%, or 12%; between 12% and 50%, 48%, 46%, 44%, 42%, 40%, 38%, 36%, 34%, 32%, 30%, 28%, 26%, 24%, 22%, 20%, 18%, 16%, or 14%; between 14% and 50%, 48%, 46%, 44%, 42%, 40%, 38%, 36%, 34%, 32%, 30%, 28%, 26%, 24%, 22%, 20%, 18%, or 16%; between 16% and 50%, 48%, 46%, 44%, 42%, 40%, 38%, 36%, 34%, 32%, 30%, 28%, 26%, 24%, 22%, 20%, or 18%; between 18% and 50%, 48%, 46%, 44%, 42%, 40%, 38%, 36%, 34%, 32%, 30%, 28%, 26%, 24%, 22%, or 20%; between 20% and 50%, 48%, 46%, 44%, 42%, 40%, 38%, 36%, 34%, 32%, 30%, 28%, 26%, 24%, or 22%; between 22% and 50%, 48%, 46%, 44%, 42%, 40%, 38%, 36%, 34%, 32%, 30%, 28%, 26%, or 24%; between 24% and 50%, 48%, 46%, 44%, 42%, 40%, 38%, 36%, 34%, 32%, 30%, 28%, 26%; between 26% and 50%, 48%, 46%, 44%, 42%, 40%, 38%, 36%, 34%, 32%, 30%, or 28%; between 28% and 50%, 48%, 46%, 44%, 42%, 40%, 38%, 36%, 34%, 32%, or 30%; between 30% and 50%, 48%, 46%, 44%, 42%, 40%, 38%, 36%, 34%, or 32%; between 32% and 50%, 48%, 46%, 44%, 42%, 40%, 38%, 36%, or 34%; between 34% and 50%, 48%, 46%, 44%, 42%, 40%, 38%, or 36%; between 36% and 50%, 48%, 46%, 44%, 42%, 40%, or 38%; between 38% and 50%, 48%, 46%, 44%, 42%, or 40%; between 40% and 50%, 48%, 46%, 44%, or 42%; between 42% and 50%, 48%, 46%, or 44%; between 44% and 50%, 48%, or 46%; between 46% and 50%, or 48%; or between 48% and 50% by mass of palmitoleic acid (C16:1); between 0% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, 4%, or 2%; between 2% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, or 4%; between 4% and 50%, 48%, 46%, 44%, 42%, 40%, 38%, 36%, 34%, 32%, 30%, 28%, 26%, 24%, 22%, 20%, 18%, 16%, 14%, 12%, 0%, 8%, or 6%; between 6% and 50%, 48%, 46%, 44%, 42%, 40%, 38%, 36%, 34%, 32%, 30%, 28%, 26%, 24%, 22%, 20%, 18%, 16%, 14%, 12%, 10%, or 8%; between 8% and 50%, 48%, 46%, 44%, 42%, 40%, 38%, 36%, 34%, 32%, 30%, 28%, 26%, 24%, 22%, 20%, 18%, 16%, 14%, 12%, or 10%; between 10% and 50%, 48%, 46%, 44%, 42%, 40%, 38%, 36%, 34%, 32%, 30%, 28%, 26%, 24%, 22%, 20%, 18%, 16%, 14%, or 12%; between 12% and 50%, 48%, 46%, 44%, 42%, 40%, 38%, 36%, 34%, 32%, 30%, 28%, 26%, 24%, 22%, 20%, 18%, 16%, or 14%; between 14% and 50%, 48%, 46%, 44%, 42%, 40%, 38%, 36%, 34%, 32%, 30%, 28%, 26%, 24%, 22%, 20%, 18%, or 16%; between 16% and 50%, 48%, 46%, 44%, 42%, 40%, 38%, 36%, 34%, 32%, 30%, 28%, 26%, 24%, 22%, 20%, or 18%; between 18% and 50%, 48%, 46%, 44%, 42%, 40%, 38%, 36%, 34%, 32%, 30%, 28%, 26%, 24%, 22%, or 20%; between 20% and 50%, 48%, 46%, 44%, 42%, 40%, 38%, 36%, 34%, 32%, 30%, 28%, 26%, 24%, or 22%; between 22% and 50%, 48%, 46%, 44%, 42%, 40%, 38%, 36%, 34%, 32%, 30%, 28%, 26%, or 24%; between 24% and 50%, 48%, 46%, 44%, 42%, 40%, 38%, 36%, 34%, 32%, 30%, 28%, 26%; between 26% and 50%, 48%, 46%, 44%, 42%, 40%, 38%, 36%, 34%, 32%, 30%, or 28%; between 28% and 50%, 48%, 46%, 44%, 42%, 40%, 38%, 36%, 34%, 32%, or 30%; between 30% and 50%, 48%, 46%, 44%, 42%, 40%, 38%, 36%, 34%, or 32%; between 32% and 50%, 48%, 46%, 44%, 42%, 40%, 38%, 36%, or 34%; between 34% and 50%, 48%, 46%, 44%, 42%, 40%, 38%, or 36%; between 36% and 50%, 48%, 46%, 44%, 42%, 40%, or 38%; between 38% and 50%, 48%, 46%, 44%, 42%, or 40%; between 40% and 50%, 48%, 46%, 44%, or 42%; between 42% and 50%, 48%, 46%, or 44%; between 44% and 50%, 48%, or 46%; between 46% and 50%, or 48%; or between 48% and 50% by mass of margaric acid (C17:0); between 0% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, 4%, or 2%; between 2% and 50%, 48%, 46%, 44%, 42%, 40%, 38%, 36%, 34%, 32%, 30%, 28%, 26%, 24%, 22%, 20%, 18%, 16%, 14%, 12%, 0%, 8%, 6%, or 4%; between 4% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, or 6%; between 6% and 50%, 48%, 46%, 44%, 42%, 40%, 38%, 36%, 34%, 32%, 30%, 28%, 26%, 24%, 22%, 20%, 18%, 16%, 14%, 12%, 10%, or 8%; between 8% and 50%, 48%, 46%, 44%, 42%, 40%, 38%, 36%, 34%, 32%, 30%, 28%, 26%, 24%, 22%, 20%, 18%, 16%, 14%, 12%, or 10%; between 10% and 50%, 48%, 46%, 44%, 42%, 40%, 38%, 36%, 34%, 32%, 30%, 28%, 26%, 24%, 22%, 20%, 18%, 16%, 14, or 12%; between 12% and 50%, 48%, 46%, 44%, 42%, 40%, 38%, 36%, 34%, 32%, 30%, 28%, 26%, 24%, 22%, 20%, 18%, 16%, or 14%; between 14% and 50%, 48%, 46%, 44%, 42%, 40%, 38%, 36%, 34%, 32%, 30%, 28%, 26%, 24%, 22%, 20%, 18%, or 16%; between 16% and 50%, 48%, 46%, 44%, 42%, 40%, 38%, 36%, 34%, 32%, 30%, 28%, 26%, 24%, 22%, 20%, or 18%; between 18% and 50%, 48%, 46%, 44%, 42%, 40%, 38%, 36%, 34%, 32%, 30%, 28%, 26%, 24%, 22%, or 20%; between 20% and 50%, 48%, 46%, 44%, 42%, 40%, 38%, 36%, 34%, 32%, 30%, 28%, 26%, 24%, or 22%; between 22% and 50%, 48%, 46%, 44%, 42%, 40%, 38%, 36%, 34%, 32%, 30%, 28%, 26%, or 24%; between 24% and 50%, 48%, 46%, 44%, 42%, 40%, 38%, 36%, 34%, 32%, 30%, 28%, 26%; between 26% and 50%, 48%, 46%, 44%, 42%, 40%, 38%, 36%, 34%, 32%, 30%, or 28%; between 28% and 50%, 48%, 46%, 44%, 42%, 40%, 38%, 36%, 34%, 32%, or 30%; between 30% and 50%, 48%, 46%, 44%, 42%, 40%, 38%, 36%, 34%, or 32%; between 32% and 50%, 48%, 46%, 44%, 42%, 40%, 38%, 36%, or 34%; between 34% and 50%, 48%, 46%, 44%, 42%, 40%, 38%, or 36%; between 36% and 50%, 48%, 46%, 44%, 42%, 40%, or 38%; between 38% and 50%, 48%, 46%, 44%, 42%, or 40%; between 40% and 50%, 48%, 46%, 44%, or 42%; between 42% and 50%, 48%, 46%, or 44%; between 44% and 50%, 48%, or 46%; between 46% and 50%, or 48%; or between 48% and 50% by mass of stearic acid (C18:0); or between 0% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, 4%, or 2%; between 2% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, 6%, or 4%; between 4% and 50%, 48%, 46%, 44%, 42%, 40%, 38%, 36%, 34%, 32%, 30%, 28%, 26%, 24%, 22%, 20%, 18%, 16%, 14%, 12%, 10%, 8%, or 6%; between 6% and 50%, 48%, 46%, 44%, 42%, 40%, 38%, 36%, 34%, 32%, 30%, 28%, 26%, 24%, 22%, 20%, 18%, 16%, 14%, 12%, 10%, or 8%; between 8% and 50%, 48%, 46%, 44%, 42%, 40%, 38%, 36%, 34%, 32%, 30%, 28%, 26%, 24%, 22%, 20%, 18%, 16%, 14%, 12%, or 10%; between 10% and 50%, 48%, 46%, 44%, 42%, 40%, 38%, 36%, 34%, 32%, 30%, 28%, 26%, 24%, 22%, 20%, 18%, 16%, 14%, or 12%; between 12% and 50%, 48%, 46%, 44%, 42%, 40%, 38%, 36%, 34%, 32%, 30%, 28%, 26%, 24%, 22%, 20%, 18%, 16%, or 14%; between 14% and 50%, 48%, 46%, 44%, 42%, 40%, 38%, 36%, 34%, 32%, 30%, 28%, 26%, 24%, 22%, 20%, 18%, or 16%; between 16% and 50%, 48%, 46%, 44%, 42%, 40%, 38%, 36%, 34%, 32%, 30%, 28%, 26%, 24%, 22%, 20%, or 18%; between 18% and 50%, 48%, 46%, 44%, 42%, 40%, 38%, 36%, 34%, 32%, 30%, 28%, 26%, 24%, 22%, or 20%; between 20% and 50%, 48%, 46%, 44%, 42%, 40%, 38%, 36%, 34%, 32%, 30%, 28%, 26%, 24%, or 22%; between 22% and 50%, 48%, 46%, 44%, 42%, 40%, 38%, 36%, 34%, 32%, 30%, 28%, 26%, or 24%; between 24% and 50%, 48%, 46%, 44%, 42%, 40%, 38%, 36%, 34%, 32%, 30%, 28%, 26%; between 26% and 50%, 48%, 46%, 44%, 42%, 40%, 38%, 36%, 34%, 32%, 30%, or 28%; between 28% and 50%, 48%, 46%, 44%, 42%, 40%, 38%, 36%, 34%, 32%, or 30%; between 30% and 50%, 48%, 46%, 44%, 42%, 40%, 38%, 36%, 34%, or 32%; between 32% and 50%, 48%, 46%, 44%, 42%, 40%, 38%, 36%, or 34%; between 34% and 50%, 48%, 46%, 44%, 42%, 40%, 38%, or 36%; between 36% and 50%, 48%, 46%, 44%, 42%, 40%, or 38%; between 38% and 50%, 48%, 46%, 44%, 42%, or 40%; between 40% and 50%, 48%, 46%, 44%, or 42%; between 42% and 50%, 48%, 46%, or 44%; between 44% and 50%, 48%, or 46%; between 46% and 50%, or 48%; or between 48% and 50% by mass of oleic acid (C18:1); or any combination thereof.

### Milk Free Fatty Acids

The milk lipid component of any of the above can consist of or comprise a single mFFA, or two or more distinct mFFAs.

The milk lipid component of any of the above can consist of or comprise one or more mFFAs that have a carbon atom number that ranges from 4 to 54.

The milk lipid component of any of the above can consist of or comprise one or more mFFAs selected from the group consisting of: butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can comprise a mFFA in protonated form, de-protonated (i.e., charged) form, or water-insoluble salt form (e.g., complexed with a divalent cation [e.g., calcium cation, magnesium cation]).

The milk lipid component of any of the above can comprise less than 5%, less than 4%, less than 3%, less than 2%, less than 1%, or less than 0.5% by mass of mFFAs.

### Milk Triglycerides

The milk lipid component of any of the above can consist of or comprise a single mTAG, or two or more distinct mTAGs.

The milk lipid component of any of the above can consist of or comprise one or more mTAGs having a fatty acid in sn1, sn2, and/or sn3 position that has a carbon atom number that ranges from 4 to 54. A suitable fatty acid can be selected from the group consisting of butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having a fatty acid in sn1, sn2, and/or sn3 position that has a carbon atom number that is even. The mTAGs can comprise at least two such fatty acids, wherein each fatty acid comprises a different even number of carbon atoms, or wherein two or all of the fatty acids comprise the same even number of carbon atoms. A suitable fatty acid can be selected from the group consisting of butyric acid (C4:0), caproic acid (C6:0), caprylic acid (C8:0), capric acid (C10:0), lauric acid (C12:0), myristic acid (C14:0), palmitic acid (C16:0), palmitoleic acid (C16:1), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having a fatty acid in sn1, sn2, and/or sn3 position that is a saturated fatty acid. The saturated fatty acid can have a carbon atom number that ranges from 4 to 54. A suitable fatty acid can be selected from the group consisting of butyric acid (C4:0), caproic acid (C6:0), caprylic acid (C8:0), capric acid (C10:0), lauric acid (C12:0), myristic acid (C14:0), palmitic acid (C16:0), and stearic acid (C18:0).

The milk lipid component of any of the above can consist of or comprise one or more mTAGs having a fatty acid in sn1, sn2, and/or sn3 position that is an unsaturated fatty acid. The unsaturated fatty acid can have a carbon atom number that ranges from 4 to 54. The unsaturated fatty acid can have a carbon atom number of 16 or 18. A suitable fatty acid can be selected from the group consisting of palmitoleic acid (C16:1), oleic acid (C18:1), linoleic acid (C18:2), linolenic acid (C18:3), and vaccenic acid (C18:1 trans11).

The milk lipid component of any of the above can consist of or comprise one or more mTAGs having a fatty acid in sn1, sn2, and/or sn3 position, wherein the mTAGs comprise at least one saturated fatty acid and at least one unsaturated fatty acid. The mTAGs can comprise two saturated fatty acids and one unsaturated fatty acid. The saturated and unsaturated fatty acids can have a carbon atom number that ranges from 4 to 54.

The milk lipid component of any of the above can consist of or comprise one or more mTAGs having between 34 and 40 carbon atoms, or one or more mTAGs having between 48 and 52 carbon atoms, or mixtures thereof.

The milk lipid component of any of the above can consist of or comprise one or more mTAGs having a fatty acid in sn1, sn2, and/or sn3 position that has a carbon atom number of less than 50, less than 40, less than 30, or less than 24.

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn3 position that has a carbon atom number that ranges from 4 to 10. A suitable fatty acid can be selected from the group consisting of butyric acid (C4:0), caproic acid (C6:0), and caprylic acid (C8:0).

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn2 position that has a carbon atom number that ranges from 8 to 16. A suitable fatty acid can be selected from the group consisting of caprylic acid (C8:0), capric acid (C10:0), lauric acid (C12:0), myristic acid (C14:0), pentadecanoic acid (C15:0), and palmitic acid (C16:0).

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn1 and/or sn3 position that has a carbon atom number that ranges from 16 to 54. A suitable fatty acid can be selected from the group consisting of palmitic acid (C16:0), palmitoleic acid (C16:1), margaric acid (C17:0), stearic acid (C18:0), and oleic acid (C18:1).

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn1 position that has a carbon atom number that ranges from 16 to 54, and a fatty acid (e.g., a saturated fatty acid, an unsaturated fatty acid, a cis-monoenoic fatty acid, a trans-monoenoic fatty acid) in sn2 position that has a carbon atom number that ranges from 8 to 16. Suitable fatty acids can be selected from the group consisting of caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn1 position that has a carbon atom number that ranges from 16 to 54, and a fatty acid (e.g., a saturated fatty acid, an unsaturated fatty acid, a cis-monoenoic fatty acid, a trans-monoenoic fatty acid) in sn3 position that has a carbon atom number that ranges from 4 to 10. Suitable fatty acids can be selected from the group consisting of butyric acid (C4:0), caproic acid (C6:0), caprylic acid (C8:0), capric acid (C10:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn2 position that has a carbon atom number that ranges from 8 to 16, and a fatty acid (e.g., a saturated fatty acid, an unsaturated fatty acid, a cis-monoenoic fatty acid, a trans-monoenoic fatty acid) in sn3 position that has a carbon atom number that ranges from 4 to 10. Suitable fatty acids can be selected from the group consisting of butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), and palmitoleic acid (C16:1).

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn2 position that has a carbon atom number that ranges from 16 to 54, and a fatty acid (e.g., a saturated fatty acid, an unsaturated fatty acid, a cis-monoenoic fatty acid, a trans-monoenoic fatty acid) in sn3 position that has a carbon atom number that ranges from 4 to 10. Suitable fatty acids can be selected from the group consisting of butyric acid (C4:0), caproic acid (C6:0), caprylic acid (C8:0), capric acid (C10:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn1 position that has a carbon atom number that ranges from 8 to 16, and a fatty acid (e.g., a saturated fatty acid, an unsaturated fatty acid, a cis-monoenoic fatty acid, a trans-monoenoic fatty acid) in sn3 position that has a carbon atom number that ranges from 4 to 10. Suitable fatty acids can be selected from the group consisting of butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), and palmitoleic acid (C16:1).

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn1 position that has a carbon atom number that ranges from 16 to 54, a fatty acid (e.g., a saturated fatty acid, an unsaturated fatty acid, a cis-monoenoic fatty acid, a trans-monoenoic fatty acid) in sn2 position that has a carbon atom number that ranges from 8 to 16, and a fatty acid (e.g., a saturated fatty acid, an unsaturated fatty acid, a cis-monoenoic fatty acid, a trans-monoenoic fatty acid) in sn3 position that has a carbon atom number that ranges from 4 to 10. Suitable fatty acids can be selected from the group consisting of butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having two fatty acids (e.g., saturated fatty acids, unsaturated fatty acids, cis-monoenoic fatty acids, trans-monoenoic fatty acids, or combinations thereof) in sn1 and sn2 positions that each have a carbon atom number that ranges from 16 to 54, and a fatty acid (e.g., a saturated fatty acid, an unsaturated fatty acid, a cis-monoenoic fatty acid, a trans-monoenoic fatty acid) in sn3 position that has a carbon atom number that ranges from 4 to 10. Suitable fatty acids can be selected from the group consisting of butyric acid (C4:0), caproic acid (C6:0), caprylic acid (C8:0), capric acid (C10:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn1 position that has a carbon atom number that ranges from 8 to 16, a fatty acid (e.g., a saturated fatty acid, an unsaturated fatty acid, a cis-monoenoic fatty acid, a trans-monoenoic fatty acid) in sn2 position that has a carbon atom number that ranges from 16 to 54, and a fatty acid (e.g., a saturated fatty acid, an unsaturated fatty acid, a cis-monoenoic fatty acid, a trans-monoenoic fatty acid) in sn3 position that has a carbon atom number that ranges from 4 to 10. Suitable fatty acids can be selected from the group consisting of butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn1 position that has a carbon atom number that ranges from 8 to 16, a fatty acid (e.g., a saturated fatty acid, an unsaturated fatty acid, a cis-monoenoic fatty acid, a trans-monoenoic fatty acid) in sn2 position that has a carbon atom number that ranges from 8 to 16, and a fatty acid (e.g., a saturated fatty acid, an unsaturated fatty acid, a cis-monoenoic fatty acid, a trans-monoenoic fatty acid) in sn3 position that has a carbon atom number that ranges from 4 to 10. Suitable fatty acids can be selected from the group consisting of butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), and palmitoleic acid (C16:1).

The milk lipid component of any of the above can consist of or comprise one or more mTAGs having fatty acids (e.g., saturated fatty acids, unsaturated fatty acids, cis-monoenoic fatty acids, trans-monoenoic fatty acids, or combination thereof) in sn1, sn2, and sn3 positions that each have a carbon atom number that ranges from 16 to 54. Suitable fatty acids can be selected from the group consisting of palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having fatty acids (e.g., saturated fatty acids, unsaturated fatty acids, cis-monoenoic fatty acids, trans-monoenoic fatty acids, or combinations thereof) in sn1 and sn2 positions that each have a carbon atom number that ranges from 16 to 54, and a fatty acid (e.g., a saturated fatty acid, an unsaturated fatty acid, a cis-monoenoic fatty acid, a trans-monoenoic fatty acid) in sn3 position that has a carbon atom number that ranges from 8 to 16. Suitable fatty acids can be selected from the group consisting of caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having fatty acids (e.g., saturated fatty acids, unsaturated fatty acids, cis-monoenoic fatty acids, trans-monoenoic fatty acids, or combinations thereof) in sn2 and sn3 positions that each have a carbon atom number that ranges from 8 to 16, and a fatty acid (e.g., a saturated fatty acid, an unsaturated fatty acid, a cis-monoenoic fatty acid, a trans-monoenoic fatty acid) in sn1 position that has a carbon atom number that ranges from 16 to 54. Suitable fatty acids can be selected from the group consisting of caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn1 position that has a carbon atom number that ranges from 16 to 54, a fatty acid (e.g., a saturated fatty acid, an unsaturated fatty acid, a cis-monoenoic fatty acid, a trans-monoenoic fatty acid) in sn2 position that has a carbon atom number that ranges from 4 to 10, and a fatty acid (e.g., a saturated fatty acid, an unsaturated fatty acid, a cis-monoenoic fatty acid, a trans-monoenoic fatty acid) in sn3 position that has a carbon atom number that ranges from 8 to 16. Suitable fatty acids can be selected from the group consisting of butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having a fatty acid (e.g., a saturated fatty acid, an unsaturated fatty acid, a cis-monoenoic fatty acid, a trans-monoenoic fatty acid) in sn1 position that has a carbon atom number that ranges from 16 to 54, and fatty acids (e.g., saturated fatty acids, unsaturated fatty acids, cis-monoenoic fatty acids, trans-monoenoic fatty acids, or combinations thereof) in sn2 and sn3 positions that each have a carbon atom number that ranges from 4 to 10. Suitable fatty acids can be selected from the group consisting of butyric acid (C4:0), caproic acid (C6:0), caprylic acid (C8:0), capric acid (C10:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mTAGs having a structure selected from the group consisting of C16:0-C14:0-C4:0, C14:0-C16:0-C18:1, C16:0-C16:0-C18:1, C16:0-C16:0-C4:0, C18:1-C16:0-C4:0, C18:1(n-9)-C16:0-C14:0, C18:1(n-9)-C16:0-C16:0, C18:1(n-9)-C16:0-C4:0, C16:0-C18:1-C18:1, C4:0-C14:0-C16:0, C16:0-C16:0-C16:1, C4:0-C16:0-C16:0, C4:0-C16:0-C18:0, C4:0-C16:0-C18:1, C6:0-C14:0-C16:0, C14:0-C18:0-C18:1, and combinations thereof.

The milk lipid component of any of the above can consist of or comprise between 50% and 100%, 95%, 90%, 85%, 80%, 75%, 70%, 65%, 60%, or 55%; between 55% and 100%, 95%, 90%, 85%, 80%, 75%, 70%, 65%, or 60%; between 60% and 100%, 95%, 90%, 85%, 80%, 75%, 70%, or 65%; between 65% and 100%, 95%, 90%, 85%, 80%, 75%, or 70%; between 70% and 100%, 95%, 90%, 85%, 80%, or 75%; between 75% and 100%, 95%, 90%, 85%, or 80%; between 80% and 100%, 95%, 90%, or 85%; between 85% and 100%, 95%, or 90%; between 90% and 100%, or 95%; or between 95% and 100% by mass of mTAGs comprising saturated fatty acids with a carbon chain length of between 4 and 18. Suitable fatty acids can be selected from the group consisting of butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), margaric acid (C17:0), and stearic acid (C18:0).

The milk lipid component of any of the above can consist of or comprise one or more structured mTAGs in which naturally occurring fatty acids in sn3 positions having a carbon atom number that ranges from 16 to 54 are replaced by fatty acids having a carbon atom number that ranges from 4 to 10. Suitable fatty acids having a carbon atom number that ranges from 4 to 10 can be selected from the group consisting of butyric acid (C4:0), caproic acid (C6:0), caprylic acid (C8:0), and capric acid (C10:0).

### Milk Phospholipids

The milk lipid component of any of the above can consist of or comprise a single mPL, or two or more distinct mPLs.

The milk lipid component of any of the above can consist of or comprise one or more mPLs having a phosphate group in sn3 position that is selected from the group consisting of phosphatidylcholine (PC), phosphatidylinositol (PI), phosphatidylserine (PS), and phosphatidylethanolamine (PE); and a fatty acid in sn1 and/or sn2 position that has a carbon atom number that ranges from 4 to 54. A suitable fatty acid can be selected from the group consisting of butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mPLs having a phosphate group in sn3 position that is selected from the group consisting of PC, PI, PS, and PE; and a fatty acid in sn1 and/or sn2 position that has a carbon atom number that is even. The fatty acid in sn1 and/or sn2 position can have a carbon atom number that is even. When present at both the sn1 and sn2 position, each fatty acid can have a different or identical even number of carbon atoms. The fatty acid in sn1 and/or sn2 position can have a carbon atom number that ranges from 4 to 54. A suitable fatty acid can be selected from the group consisting of butyric acid (C4:0), caproic acid (C6:0), caprylic acid (C8:0), capric acid (C10:0), lauric acid (C12:0), myristic acid (C14:0), palmitic acid (C16:0), palmitoleic acid (C16:1), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mPLs having a phosphate group in sn3 position that is selected from the group consisting of PC, PI, PS, and PE; and a fatty acid in sn1 and/or sn2 position that is a saturated fatty acid. The saturated fatty acid can have a carbon atom number that ranges from 4 to 54. A suitable saturated fatty acid can be selected from the group consisting of butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), margaric acid (C17:0), and stearic acid (C18:0).

The milk lipid component of any of the above can consist of or comprise one or more mPLs having a phosphate group in sn3 position that is selected from the group consisting of PC, PI, PS, and PE; and a fatty acid in sn1 and/or sn2 position that is an unsaturated fatty acid. The unsaturated fatty acid can have a carbon atom number that ranges from 4 to 54. A suitable unsaturated fatty acid can be selected from the group consisting of palmitoleic acid (C16:1), oleic acid (C18:1), linoleic acid (C18:2), linolenic acid (C18:3), and vaccenic acid (C18:1 trans11).

The milk lipid component of any of the above can consist of or comprise one or more mPLs having a phosphate group in sn3 position that is selected from the group consisting of PC, PI, PS, and PE; and fatty acids in sn1 and sn2 positions, wherein one of the fatty acids is a saturated fatty acid and the other fatty acid is an unsaturated fatty acid. The saturated and unsaturated fatty acids can have a carbon atom number that ranges from 4 to 54.

The milk lipid component of any of the above can consist of or comprise one or more mPLs having a phosphate group in sn3 position that is selected from the group consisting of PC, PI, PS, and PE; and a fatty acid in sn1 and/or sn2 position that has a carbon atom number of less than 50, less than 40, less than 30, or less than 24.

The milk lipid component of any of the above can consist of or comprise one or more mPLs having a phosphate group in sn3 position that is selected from the group consisting of PC, PI, PS, and PE; and a fatty acid (e.g., a saturated fatty acid, an unsaturated fatty acid, a cis-monoenoic fatty acid, a trans-monoenoic fatty acid) in sn1 and/or sn2 position that has a carbon atom number that ranges from 8 to 16. A suitable fatty acid can be selected from the group consisting of caprylic acid (C8:0), capric acid (C10:0), lauric acid (C12:0), myristic acid (C14:0), pentadecanoic acid (C15:0), palmitic acid (C16:0), palmitoleic acid (C16:1).

The milk lipid component of any of the above can consist of or comprise one or more mPLs having a phosphate group in sn3 position that is selected from the group consisting of PC, PI, PS, and PE; and a fatty acid (e.g., a saturated fatty acid, an unsaturated fatty acid, a cis-monoenoic fatty acid, a trans-monoenoic fatty acid) in sn1 and/or sn2 position that has a carbon atom number that ranges from 16 to 54. A suitable fatty acid can be selected from the group consisting of palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mPLs having a phosphate group in sn3 position that is selected from the group consisting of PC, PI, PS, and PE; and a fatty acid (e.g., a saturated fatty acid, an unsaturated fatty acid, a cis-monoenoic fatty acid, a trans-monoenoic fatty acid) in sn1 position that has a carbon atom number that ranges from 16 to 54, and a fatty acid (e.g., a saturated fatty acid, an unsaturated fatty acid, a cis-monoenoic fatty acid, a trans-monoenoic fatty acid) in sn2 position that has a carbon atom number that ranges from 8 to 16. Suitable fatty acids can be selected from the group consisting of caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise one or more mPLs having a phosphate group in sn3 position that is selected from the group consisting of PC, PI, PS, and PE; and a fatty acid (e.g., a saturated fatty acid, an unsaturated fatty acid, a cis-monoenoic fatty acid, a trans-monoenoic fatty acid) in sn1 position that has a carbon atom number that ranges from 8 to 16, and a fatty acid (e.g., a saturated fatty acid, an unsaturated fatty acid, a cis-monoenoic fatty acid, a trans-monoenoic fatty acid) in sn2 position that has a carbon atom number that ranges from 16 to 54. Suitable fatty acids can be selected from the group consisting of caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

The milk lipid component of any of the above can consist of or comprise between 50% and 100%, 95%, 90%, 85%, 80%, 75%, 70%, 65%, 60%, or 55%; between 55% and 100%, 95%, 90%, 85%, 80%, 75%, 70%, 65%, or 60%; between 60% and 100%, 95%, 90%, 85%, 80%, 75%, 70%, or 65%; between 65% and 100%, 95%, 90%, 85%, 80%, 75%, or 70%; between 70% and 100%, 95%, 90%, 85%, 80%, or 75%; between 75% and 100%, 95%, 90%, 85%, or 80%; between 80% and 100%, 95%, 90%, or 85%; between 85% and 100%, 95%, or 90%; between 90% and 100%, or 95%; or between 95% and 100% by mass of mPLs.

### Melting Profile

The milk lipid component of any of the above can consist of or comprise a low-melting fraction. The term “low-melting fraction” as used herein refers to one or more lipids that have a melting point of between −25° C. and 10° C.

The milk lipid component of any of the above can consist of or comprise a medium-melting fraction. The term “medium-melting fraction” as used herein refers to one or more lipids that have a melting point of between 10° C. and 20° C.

The milk lipid component of any of the above can consist of or comprise a high-melting fraction. The term “high-melting fraction” as used herein refers to one or more lipids that have a melting point of between 20° C. and 45° C.

The milk lipid component of any of the above can comprise between 0% and 100%, 90%, 80%, 70%, 60%, 50%, 40%, 30%, 20%, or 10%; between 10% and 100%, 90%, 80%, 70%, 60%, 50%, 40%, 30%, or 20%; between 20% and 100%, 90%, 80%, 70%, 60%, 50%, 40%, or 30%; between 30% and 100%, 90%, 80%, 70%, 60%, 50%, or 40%; between 40% and 100%, 90%, 80%, 70%, 60%, or 50%; between 50% and 100%, 90%, 80%, 70%, or 60%; between 60% and 100%, 90%, 80%, or 70%; between 70% and 100%, 90%, or 80%; between 80% and 100%, or 90%; or between 90% and 100% by mass of a low-melting fraction.

The milk lipid component of any of the above can comprise between 0% and 100%, 90%, 80%, 70%, 60%, 50%, 40%, 30%, 20%, or 10%; between 10% and 100%, 90%, 80%, 70%, 60%, 50%, 40%, 30%, or 20%; between 20% and 100%, 90%, 80%, 70%, 60%, 50%, 40%, or 30%; between 30% and 100%, 90%, 80%, 70%, 60%, 50%, or 40%; between 40% and 100%, 90%, 80%, 70%, 60%, or 50%; between 50% and 100%, 90%, 80%, 70%, or 60%; between 60% and 100%, 90%, 80%, or 70%; between 70% and 100%, 90%, or 80%; between 80% and 100%, or 90%; or between 90% and 100% by mass of a medium-melting fraction.

The milk lipid component of any of the above can comprise between 0% and 100%, 90%, 80%, 70%, 60%, 50%, 40%, 30%, 20%, or 10%; between 10% and 100%, 90%, 80%, 70%, 60%, 50%, 40%, 30%, or 20%; between 20% and 100%, 90%, 80%, 70%, 60%, 50%, 40%, or 30%; between 30% and 100%, 90%, 80%, 70%, 60%, 50%, or 40%; between 40% and 100%, 90%, 80%, 70%, 60%, or 50%; between 50% and 100%, 90%, 80%, 70%, or 60%; between 60% and 100%, 90%, 80%, or 70%; between 70% and 100%, 90%, or 80%; between 80% and 100%, or 90%; or between 90% and 100% by mass of a high-melting fraction.

The milk lipid component of any of the above can consist of a low-melting fraction and a medium-melting fraction.

The milk lipid component of any of the above can consist of a low-melting fraction and a high-melting fraction.

The milk lipid component of any of the above can consist of a medium-melting fraction and a high-melting fraction.

The milk lipid component of any of the above can consist of a low-melting fraction, a medium-melting fraction, and a high-melting fraction.

The milk lipid component of any of the above can consist of or comprise between 45% and 55% by mass of a low-melting fraction, between 25% and 45% by mass of a medium-melting fraction, and between 1% and 20% of a high-melting fraction.

The milk lipid component of any of the above can comprise a fraction of solid lipid at ambient temperature and conditions (i.e., 20° C.-30° C. and 0.95-1.05 atm).

The milk lipid component of any of the above can comprise a fraction of solid lipid at body temperature and conditions (i.e., 36° C.-38° C. and 0.95-1.05 atm).

### Flavor/Aroma Profile

The milk lipid component of any of the above can have a flavor/aroma profile that is similar to that of a mammal-produced milk fat as determined by an expert human sensory panel.

The milk lipid component of any of the above can have a flavor/aroma profile that is bland (i.e., does not score highly on any flavor/aroma note in a sensory analysis).

### Emulsifying Potential

The milk lipid component of any of the above can have an emulsifying potential that is similar to that of a mammal-produced milk fat. Methods for determining emulsifying potential are known in the art.

The milk lipid component of any of the above can have an emulsifying potential that is greater than that of a plant oil (e.g., soybean oil).

### Lipid Component

In another aspect, provided herein is a lipid component that consists of the milk lipid component of any of the above and an optional non-milk lipid component, and that can impart a desirable attribute on a composition. The term “non-milk lipid component” as used herein refers to a component that consists of non-milk lipids.

The lipid component of any of the above can comprise at least 0.001%, at least 0.01%, at least 0.1%, at least 1%, at least 10%, at least 20%, at least 30%, at least 40%, at least 50%, at least 60%, at least 70%, at least 80%, at least 90%, at least 95%, or at least 99%; or between 0.001% and 100%, 99%, 95%, 90%, 80%, 70%, 60%, 50%, 40%, 30%, 20%, 15%, 10%, 9%, 8%, 7%, 6%, 5%, 4%, 3%, 2%, 1%, 0.1%, or 0.01%; between 0.01% and 100%, 99%, 95%, 90%, 80%, 70%, 60%, 50%, 40%, 30%, 20%, 15%, 10%, 9%, 8%, 7%, 6%, 5%, 4%, 3%, 2%, 1%, or 0.1%; between 0.1% and 100%, 99%, 95%, 90%, 80%, 70%, 60%, 50%, 40%, 30%, 20%, 15%, 10%, 9%, 8%, 7%, 6%5, 5% 4%, 3%, 2%, or 1%; between 1% and 100%, 99%, 95%, 90%, 80%, 70%, 60%, 50%, 40%, 30%, 20%, 15%, 10%, 9%, 8%, 7%, 6%5, 5% 4%, 3%, or 2%; between 2% and 100%, 99%, 95%, 90%, 80%, 70%, 60%, 50%, 40%, 30%, 20%, 15%, 10%, 9%, 8%, 7%, 6%, 5%, 4%, or 3%; between 3% and 100%, 99%, 95%, 90%, 80%, 70%, 60%, 50%, 40%, 30%, 20%, 15%, 10%, 9%, 8%, 7%, 6%, 5%, or 4%; between 4% and 100%, 99%, 95%, 90%, 80%, 70%, 60%, 50%, 40%, 30%, 20%, 15%, 10%, 9%, 8%, 7%, 6%, or 5%; between 5% and 100%, 99%, 95%, 90%, 80%, 70%, 60%, 50%, 40%, 30%, 20%, 15%, 10%, 9%, 8%, 7%, or 6%; between 6% and 100%, 99%, 95%, 90%, 80%, 70%, 60%, 50%, 40%, 30%, 20%, 15%, 10%, 9%, 8%, or 7%; between 7% and 100%, 99%, 95%, 90%, 80%, 70%, 60%, 50%, 40%, 30%, 20%, 15%, 10%, 9%, or 8%; between 8% and 100%, 99%, 95%, 90%, 80%, 70%, 60%, 50%, 40%, 30%, 20%, 15%, 10%, or 9%; between 9% and 100%, 99%, 95%, 90%, 80%, 70%, 60%, 50%, 40%, 30%, 20%, 15%, or 10%; between 10% and 100%, 99%, 95%, 90%, 80%, 70%, 60%, 50%, 40%, 30%, 20%, or 15%; between 15% and 100%, 99%, 95%, 90%, 80%, 70%, 60%, 50%, 40%, 30%, or 20%; between 20% and 100%, 99%, 95%, 90%, 80%, 70%, 60%, 50%, 40%, or 30%; between 30% and 100%, 99%, 95%, 90%, 80%, 70%, 60%, 50%, or 40%; between 40% and 100%, 99%, 95%, 90%, 80%, 70%, 60%, or 50%; between 50% and 100%, 99%, 95%, 90%, 80%, 70%, or 60%; between 60% and 100%, 99%, 95%, 90%, 80%, or 70%; between 70% and 100%, 99%, 95%, 90%, or 80%; between 80% and 100%, 99%, 95%, or 90%; between 90% and 100%, 99%, or 95%; between 95% and 100% or 99%; or between 99% and 100% by mass of the milk lipid component.

The lipid component of any of the above can comprise between 0.01% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0.05%; between 0.05% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, %1%, 0.9%, 0.8%, 0.7%, 0.6%, 0.5%, 0.4%, 0.3%, 0.2%, or 0.1%; between 0.1% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, or 0.2%; between 0.2% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, or 0.3%; between 0.3% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%1, %, 0.9%, 0.8%, 0.7%, 0.6%, 0.5%, or 0.4%; between 0.4% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, or 0.5%; between 0.5% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, or 0.6%; between 0.6% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, or 0.7%; between 0.7% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, or 0.8%; between 0.8% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0.9%; between 0.9% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, or 1%; between 1% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, or 2%; between 2% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, or 3%; between 3% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, or 4%; between 4% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, or 5%; between 5% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, or 6%; between 6% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, or 7%; between 7% and 90%, 85%, 80%, 75%, 70%, 65%, 60%, 55%, 50%, 45%, 40%, 35%, 30%, 25%, 20%, 15%, 14%, 13%, 12%, 11%, 10%, 9%, or 8%; between 8% and 90%, 85%, 80%, 75%, 70%, 65%, 60%, 55%, 50%, 45%, 40%, 35%, 30%, 25%, 20%, 15%, 14%, 13%, 12%, 11%, 10%, or 9%; between 9% and 90%, 85%, 80%, 75%, 70%, 65%, 60%, 55%, 50%, 45%, 40%, 35%, 30%, 25%, 20%, 15%, 14%, 13%, 12%, 11%, or 10%; between 10% and 90%, 85%, 80%, 75%, 70%, 65%, 60%, 55%, 50%, 45%, 40%, 35%, 30%, 25%, 20%, 115%, 14%, 13%, 12%, or 11%; between 11% and 90%, 85%, 80%, 75%, 70%, 65%, 60%, 55%, 50%, 45%, 40%, 35%, 30%, 25%, 20%, 15%, 14%, 13%, or 12%; between 12% and 90%, 85%, 80%, 75%, 70%, 65%, 60%, 55%, 50%, 45%, 40%, 35%, 30%, 25%, 20%, 15%, 14%, or 13%; between 13% and 90%, 85%, 80%, 75%, 70%, 65%, 60%, 55%, 50%, 45%, 40%, 35%, 30%, 25%, 20%, 115%, or 14%; between 14% and 90%, 85%, 80%, 75%, 70%, 65%, 60%, 55%, 50%, 45%, 40%, 35%, 30%, 25%, 20%, or 15%; between 15% and 90%, 85%, 80%, 75%, 70%, 65%, 60%, 55%, 50%, 45%, 40%, 35%, 30%, 25%, or 20%; between 20% and 90%, 85%, 80%, 75% 70%, 65%, 60%, 55%, 50%, 45%, 40%, 35% 30%, or 25%; between 25% and 90%, 85%, 80%, 75%, 70%, 65%, 60%, 55%, 50%, 45%, 40%, 35%, or 30%; between 30% and 90%, 85%, 80%, 75%, 70%, 65%, 60%, 55%, 50%, 45%, 40%, or 35%; between 35% and 90%, 85%, 80%, 75%, 70%, 65%, 60%, 55%, 50%, 45%, or 40%; between 40% and 90%, 85%, 80%, 75%, 70%, 65%, 60%, 55%, 50%, or 45%; between 44% and 90%, 85%, 80%, 75%, 70%, 65%, 60%, 55%, or 50%; between 50% and 90%, 85%, 80%, 75%, 70%, 65%, 60%, or 55%; between 55% and 90%, 85%, 80%, 75%, 70%, 65%, or 60%; between 60% and 90%, 85%, 80%, 75%, 70%, or 65%; between 65% and 90%, 85%, 80%, 75%, or 70%; between 70% and 90%, 85%, 80%, or 75%; between 75% and 90%, 85%, or 80%; between 80% and 90%, or 85%; or between 85% and 90% by mass of the non-milk lipid component.

The lipid component of any of the above can comprise a milk lipid component and a non-milk lipid component at a mass ratio of between 100 to 1 and 1 to 100 (e.g., 100 to 1, 50 to 1, 40 to 1, 30 to 1, 20 to 1, 10 to 1, 9 to 1, 8 to 1, 7 to 1, 6 to 1, 5 to 1, 4 to 1, 3 to 1, 2 to 1, 1 to 1, 1 to 2, 1 to 3, 1 to 4, 1 to 5, 1 to 6, 1 to 7, 1 to 8, 1 to 9, 1 to 10, 1 to 20, 1 to 30, 1 to 40, 1 to 50, 1 to 100).

The desirable attribute that is imparted on a composition by the lipid component can be an attribute that can be imparted by a mammal-produced milk or milk fat, or by a lard or tallow.

The non-milk lipid component can consist of a single non-milk lipid, or of two or more distinct non-milk lipids.

The non-milk lipid component of any of the above can consist of or comprise one or more non-milk monoglycerides (nmMAG), one or more non-milk diglycerides (nmDAGs), one or more non-milk triglycerides (nmTAGs), one or more non-milk phospholipids (nmPLs), one or more non-milk free fatty acids (nmFFAs), or any combination thereof.

The non-milk lipid component of any of the above can consist of or comprise one or more lipids obtained from one or more sources selected from the group consisting of animals, plants, microbes (e.g., fungi [e.g. yeast, filamentous fungi], bacteria, algae [e.g., red algea, green algea, brown algea, microalgae], archaea, protozea), and combinations thereof. Non-limiting examples of such sources are disclosed herein.

The non-milk lipid component can comprise a structured non-milk lipid (e.g., a structured nmTAG).

### Composition Comprising Lipid Component

In another aspect, provided herein is a composition that comprises a lipid component according to any of the above, wherein the lipid component imparts on the composition a desirable attribute, and wherein the composition comprises no other lipid than the lipids of which the lipid component consists.

The composition can comprise between 0.001% and 90%, 80%, 70%, 60%, 50%, 40%, 30%, 20%, 15%, 10%, 9%, 8%, 7%, 6%, 5%, 4%, 3% 2%, 10%, 0.1%, or 0.01%; between 0.01% and 90%, 80%, 70%, 60%, 50%, 40%, 30%, 20%, 15%, 1%, 90%, 8%, 7%, 6%, 5%, 4%, 3%, 2%, 1%, or 0.10%; between 0.1% and 90%, 80%, 70%, 60%, 50%, 40%, 30%, 20%, 15%, 10%, 9%, 8%, 7%, 6%, 5%, 4%, 3%, 2%, or 1%; between 1% and 90%, 80%, 70%, 60%, 50%, 40%, 30%, 20%, 15%, 10%, 9%, 8%, 7%, 6%, 5%, 4%, 3%, or 2%; between 2% and 90%, 80%, 70%, 60%, 50%, 40%, 30%, 20%, 15%, 1%, 90%, 8%, 7%, 6%, 5%, 4%, or 3%; between 3% and 90%, 80%, 70%, 60%, 50%, 40%, 30%, 20%, 15%, 10%, 9%, 8%, 7%, 6%, 5%, or 4%; between 4% and 90%, 80%, 70%, 60%, 50%, 40%, 30%, 20%, 15%, 10%, 9%, 8%, 7%, 6%, or 5%; between 5% and 90%, 80%, 70%, 60%, 50%, 40%, 30%, 20%, 15%, 10%, 9%, 8%, 7%, or 6%; between 6% and 90%, 80%, 70%, 60%, 50%, 40%, 30%, 20%, 15%, 10%, 9%, 8%, or 7%; between 7% and 90%, 80%, 70%, 60%, 50%, 40%, 30%, 20%, 15%, 10%, 9%, or 8%; between 8% and 90%, 80%, 70%, 60%, 50%, 40%, 30%, 20%, 15%, 10%, or 9%; between 9% and 90%, 80%, 70%, 60%, 50%, 40%, 30%, 20%, 15%, or 10%; between 10% and 90%, 80%, 70%, 60%, 50%, 40%, 30%, 20%, or 15%; between 15% and 90%, 80%, 70%, 60%, 50%, 40%, 30%, or 20%; between 20% and 90%, 80%, 70%, 60%, 50%, 40%, or 30%; between 30% and 90%, 80%, 70%, 60%, 50%, or 40%; between 40% and 90%, 80%, 70%, 60%, or 50%; between 50% and 90%, 80%, 70%, or 60%; between 60% and 90%, 80%, or 70%; between 70% and 90%, or 80%; or between 80% and 90% by mass of the lipid component.

The composition of any of the above can further comprise a milk protein component. The term “milk protein component” as used herein refers to a component that consists of one or more whey proteins, one or more caseins, or a mixture thereof. The term implies that the milk proteins of which the milk protein component consists are the only milk proteins comprised in the composition (i.e., the composition comprises no other milk proteins other than the milk proteins of which the milk protein component consists).

The composition can comprise between 0.1% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10% 9%, 8%, 7%, 6%, 5%, 4%, 3%, 2%, 1%, 0.9%, 0.8%, 0.7%, 0.6%, 0.5%, 0.4%, 0.3%, or 0.2%; between 0.2% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, or 0.3%; between 0.3% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%1, %, 0.9%, 0.8%, 0.7%, 0.6%, 0.5%, or 0.4%; between 0.4% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, or 0.5%; between 0.5% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10% 9%, 8%, 7%, 6%, 5%, 4%, 3%, 2%1, %, 0.9%, 0.8%, 0.7%, or 0.6%; between 0.6% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, or 0.7%; between 0.7% and 99%, 95%, 90%, 85%, 80%, 75%, 70%, 65%, 60%, 55%, 50%, 45%, 40%, 35%, 30%, 25%, 20%, 15%, 14%, 13%, 12%, 1%, 10%, 9%, 8%, 7%, 6%, 5%, 4%, 3%, 2%, 1%, 0.9%, or 0.8%; between 0.8% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0.9%; between 0.9% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, or 1%; between 1% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10% 9%, 8%, 7%, 6%, 5%, 4%, 3%, or 2%; between 2% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, or 3%; between 3% and 99%, 95%, 90%, 85%, 80%, 75%, 70%, 65%, 60%, 55%, 50%, 45%, 40%, 35%, 30%, 25%, 20%, 15%, 4%, 13%, 12%, 11%, 10%, 9%, 8%, 7%, 6%, 5%, or 4%; between 4% and 99%, 95%, 90%, 85%, 80%, 75%, 70%, 65%, 60%, 55%, 50%, 45%, 40%, 35%, 30%, 25%, 20%, 15%, 4%, 113%, 12%, 1%, 10%, 9%, 8%, 7%, 6%, or 5%; between 5% and 99%, 95%, 90%, 85%, 80%, 75%, 70%, 65%, 60%, 55%, 50%, 45%, 40%, 35%, 30%, 25%, 20%, 15%, 4%, 13%, 12%, 11%, 10%, 9%, 8%, 7%, or 6%; between 6% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, or 7%; between 7% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, or 8%; between 8% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, or 9%; between 9% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, or 10%; between 10% and 99%, 95%, 90%, 85%, 80%, 75%, 70%, 65%, 60%, 55%, 50%, 45%, 40%, 35%, 30%, 25%, 20%, 15%, 14%, 13%, 12%, or 11%; between 11% and 99%, 95%, 90%, 85%, 80%, 75%, 70%, 65%, 60%, 55%, 50%, 45%, 40%, 35%, 30%, 25%, 20%, 15%, 14%, 13%, or 12%; between 12% and 99%, 95%, 90%, 85%, 80%, 75%, 70%, 65%, 60%, 55%, 50%, 45%, 40%, 35%, 30%, 25%, 20%, 15%, 14%, or 13%; between 13% and 99%, 95%, 90%, 85%, 80%, 75%, 70%, 65%, 60%, 55%, 50%, 45%, 40%, 35%, 30%, 25%, 20%, 15%, or 14%; between 14% and 99%, 95%, 90%, 85%, 80%, 75%, 70%, 65%, 60%, 55% 50%, 45%, 40%, 35%, 30%, 25%, 20%, or 15%; between 15% and 99%, 95%, 90%, 85%, 80%, 75%, 70%, 65%, 60%, 55%, 50%, 45%, 40%, 35%, 30%, 25%, or 20%; between 20% and 99%, 95%, 90%, 85%, 80%, 75%, 70%, 65%, 60%, 55% 50%, 45%, 40%, 35%, 30%, or 25%; between 25% and 100%, 95%, 90%, 85%, 80%, 75%, 70%, 65%, 60%, 55%, 50%, 45%, 40%, 35%, or 30%; between 30% and 990% 95%, 90%, 85%, 80%, 75%, 70%, 65%, 60%, 55%, 50%, 45%, 40%, or 35%; between 35% and 99%, 95%, 90%, 85%, 80%, 75%, 70%, 65%, 60%, 50%, 50%, 45%, or 40%; between 40% and 99%, 95%, 90%, 85%, 80%, 75%, 70%, 65%, 60%, 55%, 50%, or 45%; between 45% and 99%, 95%, 90%, 85%, 80%, 75%, 70%, 65%, 60%, 55%, or 50%; between 50% and 99%, 95%, 90%, 85%, 80%, 75%, 70%, 65%, 60%, or 55%; between 55% and 99%, 95%, 90%, 85%, 80%, 75%, 70%, 65%, or 60%; between 60% and 100%, 95%, 90%, 85%, 80%, 75%, 70%, or 65%; between 65% and 99%, 95%, 90%, 85%, 80%, 75%, or 70%; between 70% and 99%, 95%, 90%, 85%, 80%, or 75%; between 75% and 99%, 95%, 90%, 85%, or 80%; between 80% and 99%, 95%, 90%, or 85%; or between 85% and 99%, 95%, 90%; between 90% and 99% or 95%, or between 95% and 99% by mass of the milk protein component.

The composition of any of the above can further comprise a non-milk protein component. The term “non-milk protein component” as used herein refers to a component that consists of one or more non-milk proteins. The term implies that the non-milk proteins of which the non-milk protein component consists are the only non-milk proteins comprised in the composition (i.e., the composition comprises no other non-milk proteins other than the non-milk proteins of which the non-milk protein component consists).

The composition can comprise between 0.1% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%1, %, 0.9%, 0.8%, 0.7%, 0.6%, 0.5%, 0.4%, 0.3%, or 0.2%; between 0.2% and 99%, 95%, 90%, 85%, 80%, 75%, 70%, 65%, 60%, 55%, 50%, 45%, 40%, 35%, 30%, 25%, 20%, 1, 0%, 15%14%, %13%, %12%, 11%, 10%, 9%, 8%, 7%, 6%, 5%, 4%, 3%, 2%, 1%, 0.9%, 0.8%, 0.7%, 0.6%, 0.5%, 0.4%, or 0.3%; between 0.3% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, or 0.4%; between 0.4% and 99%, 95%, 90%, 85%, 80%, 75%, 70%, 65%, 60%, 55%, 50%, 45%, 40%, 35%, 30%, 25%, 20%, 1, 0%, 15%14%, %13%, %12%, 11%, 10%, 9%, 8%, 7%, 6%, 5%, 4%, 3%, 2%, 1%, 0.9%, 0.8%, 0.7%, 0.6%, or 0.5%; between 0.5% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, or 0.6%; between 0.6% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, or 0.7%; between 0.7% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%1, %, 0.9%, or 0.8%; between 0.8% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0.9%; between 0.9% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, or 1%; between 1% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, or 2%; between 2% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, or 3%; between 3% and 99%, 95%, 90%, 85%, 80%, 75%, 70%, 65%, 60%, 55%, 50%, 45%, 40%, 35%, 30%, 25%, 20%, 15%, 14%, 3%, 12%, 11%, 10%, 9%, 8%, 7%, 6%, 5%, or 4%; between 4% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, or 5%; between 5% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, or 6%; between 6% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, or 7%; between 7% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, or 8%; between 8% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, or 9%; between 9% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, or 10%; between 10% and 99%, 95%, 90%, 85%, 80%, 75%, 70%, 65%, 60%, 55%, 50%, 45%, 40%, 35%, 30%, 25%, 20%, 15%, 14%, 13%, 12%, or 11%; between 11% and 99%, 95%, 90%, 85%, 80%, 75%, 70%, 65%, 60%, 55%, 50%, 45%, 40%, 35%, 30%, 25%, 20%, 15%, 14%, 13%, or 12%; between 12% and 99%, 95%, 90%, 85%, 80%, 75%, 70%, 65%, 60%, 55%, 50%, 45%, 40%, 35%, 30%, 25%, 20%, 15%, 14%, or 13%; between 13% and 99%, 95%, 90%, 85%, 80%, 75%, 70%, 65%, 60%, 55%, 50%, 45%, 40%, 35%, 30%, 25%, 20%, 15%, or 14%; between 14% and 99%, 95%, 90%, 85%, 80%, 75%, 70%, 65%, 60%, 55%, 50%, 45%, 40%, 35%, 30%, 25%, 20%, or 15%; between 15% and 99%, 95%, 90%, 85%, 80%, 75%, 70%, 65%, 60%, 55%, 50%, 45%, 40%, 35%, 30%, 25%, or 20%; between 20% and 99%, 95%, 90%, 85%, 80%, 75%, 70%, 65%, 60%, 55%, 50%, 45%, 40%, 35%, 30%, or 25%; between 25% and 100%, 95%, 90%, 85%, 80%, 75%, 70%, 65%, 60%, 55%, 50%, 45%, 40%, 35%, or 30%; between 30% and 990% 95%, 90%, 85%, 80%, 75%, 70%, 65%, 60%, 55%, 50%, 45%, 40%, or 35%; between 35% and 99%, 95%, 90%, 85%, 80%, 75%, 70%, 65%, 60%, 55%, 50%, 45%, or 40%; between 40% and 99%, 95%, 90%, 85%, 80%, 75%, 70%, 65%, 60%, 55%, 50%, or 45%; between 45% and 99%, 95%, 90%, 85%, 80%, 75%, 70%, 65%, 60%, 55%, or 50%; between 50% and 99%, 95%, 90%, 85%, 80%, 75%, 70%, 65%, 60%, or 55%; between 55% and 99%, 95%, 90%, 85%, 80%, 75%, 70%, 65%, or 60%; between 60% and 100%, 95%, 90%, 85%, 80%, 75%, 70%, or 65%; between 65% and 99%, 95%, 90%, 85%, 80%, 75%, or 70%; between 70% and 99%, 95%, 90%, 85%, 80%, or 75%; between 75% and 99%, 95%, 90%, 85%, or 80%; between 80% and 99%, 95%, 90%, or 85%; or between 85% and 99%, 95%, 90%; between 90% and 99% or 95%, or between 95% and 99% by mass of the non-milk protein component.

The composition of any of the above can comprise a milk protein component and a non-milk protein component at a mass ratio of between about 100 to 1 and about 1 to 100 (e.g., about 100 to 1, about 90 to 1, about 80 to 1, about 70 to 1, about 60 to 1, about 50 to 1, about 40 to 1, about 30 to 1, about 20 to 1, about 10 to 1, about 9 to 1, about 8 to 1, about 7 to 1, about 6 to 1, about 5 to 1, about 4 to 1, about 3 to 1, about 2 to 1, about 1 to 1, about 1 to 2, about 1 to 3, about 1 to 4, about 1 to 5, about 1 to 6, about 1 to 7, about 1 to 8, about 1 to 9, about 1 to 10, about 1 to 20, about 1 to 30, about 1 to 40, about 1 to 50, about 1 to 60, about 1 to 70, about 1 to 80, about 1 to 90, or about 1 to 100).

The composition of any of the above can further comprise a milk fat globule-like structure component. The term “milk fat globule-like structure component” as used herein refers to a component that consists of one or more milk fat globule-like structures, wherein a milk fat globule-like structure comprises one or more lipids of the lipid component and one or more proteins of the milk protein component and/or the non-milk protein component.

The composition can comprise between 0.1% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, or 0.2%; between 0.2% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, or 0.3%; between 0.3% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, or 0.4%; between 0.4% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, or 0.5%; between 0.5% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, or 0.6%; between 0.6% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%1, %, 0.9%, 0.8%, or 0.7%; between 0.7% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7% 6%, 5%, 4%, 3%, 2%, 1%, 0.9%, or 0.8%; between 0.8% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0.9%; between 0.9% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, or 1%; between 1% and 99%, 95%, 90%, 85%, 80%, 75%, 70%, 65%, 60%, 55%, 50% 45%, 40%, 35%, 30%, 25%, 20%, 15%, 14%, 13%, 12%, 11%, 10%, 9%, 8%, 7%, 6%, 5%, 4%, 3%, or 2%; between 2% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, or 3%; between 3% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, or 4%; between 4% and 99%, 95%, 90%, 85%, 80%, 75%, 70%, 65%, 60%, 55%, 50%, 45%, 40%, 35%, 30%, 25%, 20%, 15%, 4%, 13%, 12%, 11%, 10%, 9%, 8%, 7%, 6%, or 5%; between 5% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, or 6%; between 6% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, or 7%; between 7% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, or 8%; between 8% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, 10%, or 9%; between 9% and 99%, 95%, 90%, 85%, 80%, 75%, 70%, 65%, 60%, 55%, 50%, 45%, 40%, 35%, 30%, 25%, 20%, 15%, 14%, 13%, 12%, 11%, or 10%; between 10% and 99%, 95%, 90%, 85%, 80%, 75%, 70%, 65%, 60%, 55%, 50%, 45%, 40%, 35%, 30%, 25%, 20%, 15%, 14%, 13%, 12%, or 11%; between 11% and 99%, 95%, 90%, 85%, 80%, 75%, 70%, 65%, 60%, 55%, 50%, 45%, 40%, 35%, 30%, 25%, 20%, 15%, 14%, 13%, or 12%; between 12% and 99%, 95%, 90%, 85%, 80%, 75%, 70%, 65%, 60%, 55%, 50%, 45%, 40%, 35%, 30%, 25%, 20%, 15%, 14%, or 13%; between 13% and 99%, 95%, 90%, 85%, 80%, 75%, 70%, 65%, 60%, 55%, 50%, 45%, 40%, 35%, 30%, 25%, 20%, 15%, or 14%; between 14% and 99%, 95%, 90%, 85%, 80%, 75%, 70%, 65%, 60%, 55%, 50%, 45%, 40%, 35%, 30%, 25%, 20%, or 15%; between 15% and 99%, 95%, 90%, 85%, 80%, 75%, 70%, 65%, 60%, 55%, 50%, 45%, 40%, 35%, 30%, 25%, or 20%; between 20% and 99%, 95%, 90%, 85%, 80%, 75%, 70%, 65%, 60%, 55%, 50%, 45%, 40%, 35%, 30%, or 25%; between 25% and 100%, 95%, 90%, 85%, 80%, 75%, 70%, 65%, 60%, 55%, 50%, 45%, 40%, 35%, or 30%; between 30% and 99%, 95%, 90%, 85%, 80%, 75%, 70%, 65%, 60%, 55%, 50%, 45%, 40%, or 35%; between 35% and 99%, 95%, 90%, 85%, 80%, 75%, 70%, 65%, 60%, 55%, 50%, 45%, or 40%; between 40% and 99%, 95%, 90%, 85%, 80%, 75%, 70%, 65%, 60%, 55%, 50%, or 45%; between 45% and 99%, 95%, 90%, 85%, 80%, 75%, 70%, 65%, 60%, 55%, or 50%; between 50% and 99%, 95%, 90%, 85%, 80%, 75%, 70%, 65%, 60%, or 55%; between 55% and 99%, 95%, 90%, 85%, 80%, 75%, 70%, 65%, or 60%; between 60% and 100%, 95%, 90%, 85%, 80%, 75%, 70%, or 65%; between 65% and 99%, 95%, 90%, 85%, 80%, 75%, or 70%; between 70% and 99%, 95%, 90%, 85%, 80%, or 75%; between 75% and 99%, 95%, 90%, 85%, or 80%; between 80% and 99%, 95%, 90%, or 85%; or between 85% and 99%, 95%, 90%; between 90% and 99% or 95%, or between 95% and 99% by mass of the milk fat globule-like structure component.

The composition of any of the above can further comprise one or more other ingredients, as described below. The composition can comprise between 0.001% and 10% by mass of any one or of all such other ingredients.

The composition of any of the above can be essentially free of or comprise 2% or less by mass of one or more components obtained from an animal (i.e., components that are native to an animal; an animal lipid, an animal protein).

The composition of any of the above can be essentially free of at least one compound found in a mammal-produced milk; or can comprise a lower concentration of at least one compound found in a mammal-produced milk. Non-limiting examples of such compounds include lactose, saturated fat, trans fatty acids or fatty acids, cholesterol, all native milk proteins, and all native milk lipids. The composition can be essentially free of at least one lipid found a mammal-produced milk.

At standard ambient temperature and conditions (i.e., 20° C.-30° C. and 0.95-1.05 atm), the composition according to any of the above can be a fluid, semi-solid, solid, or powder.

The composition of any of the above can be a powder that comprises a moisture content of less than 20%, less than 15%, less than 10%, less than 7%, less than 5%, less than 3%, or less than 1%; or between 0.1% and 20%, 15%, 10%, 5%, or 1%; between 1% and 20%, 15%, 10%, or 5%; between 5% and 20%, 15%, or 10%; between 10% and 20%, or 15%; or between 15% and 20%.

The composition of any of the above can be an emulsion.

The composition of any of the above can be an oil-in-water emulsion comprising a lipid dispersed phase and an aqueous continuous phase, wherein the lipid component according to any of the above is comprised in the dispersed phase.

The composition of any of the above can be a water-in-oil emulsion comprising a lipid continuous phase and an aqueous dispersed phase, wherein the lipid component according to any of the above is comprised in the continuous phase.

The composition of any of the above can be an emulsion comprising dispersed phase droplets having an average diameter of between 0.1 μm and 15 μm, 14 μm, 13 μm, 12 μm, 11 μm, 10 μm, 9 μm, 8 μm, 7 μm, 6 μm, 5 μm, 4 μm, 3 μm, 2 μm, 1 μm, or 0.5 μm; between 0.5 μm and 15 μm, 14 μm, 13 μm, 12 μm, 11 μm, 10 μm, 9 μm, 8 μm, 7 μm, 6 μm, 5 μm, 4 μm, 3 μm, 2 μm, or 1 μm; between 1 μm and 15 μm, 14 μm, 13 μm, 12 μm, 11 μm, 10 m, 9 m, 8 μm, 7 m, 6 μm, 5 μm, 4 μm, 3 μm, or 2 μm; between 2 μm and 15 μm, 14 μm, 13 μm, 12 μm, 11 μm, 10 μm, 9 μm, 8 μm, 7 μm, 6 μm, 5 μm, 4 μm, or 3 μm; between 3 μm and 15 μm, 14 μm, 13 μm, 12 μm, 11 μm, 10 μm, 9 μm, 8 μm, 7 μm, 6 μm, 5 μm, or 4 μm; between 4 μm and 15 μm, 14 μm, 13 μm, 12 μm, 11 μm, 10 μm, 9 μm, 8 μm, 7 μm, 6 μm, or 5 μm; between 5 μm and 15 μm, 14 μm, 13 μm, 12 μm, 11 μm, 10 μm, 9 μm, 8 μm, 7 μm, or 6 μm; between 6 μm and 15 μm, 14 μm, 13 μm, 12 μm, 11 μm, 10 μm, 9 μm, 8 μm, or 7 μm; between 7 μm and 15 μm, 14 μm, 13 μm, 12 μm, 11 μm, 10 μm, 9 μm, or 8 μm; between 8 μm and 15 μm, 14 μm, 13 μm, 12 μm, 11 μm, 10 μm, or 9 μm; between 9 μm and 15 μm, 14 μm, 13 μm, 12 μm, 11 μm, or 10 μm; between 10 m and 15 μm, 14 μm, 13 μm, 12 μm, or 11 μm; between 11 μm and 15 μm, 14 μm, 13 μm, or 12 μm; between 12 μm and 15 μm, 14 μm, or 13 μm; between 13 μm and 15 μm, or 14 μm; or between 14 μm and 15 μm.

The composition of any of the above can be an emulsion comprising dispersed phase droplets that are engulfed in a membrane.

### Optional Milk Protein Component

The optional milk protein component can consist of one or more (e.g., two, three, four, five, six, seven, eight, nine, ten, or more) milk proteins (e.g., one or more whey proteins [e.g., a β-lactoglobulin, a α-lactalbumin, a mixture of a β-lactoglobulin and a α-lactalbumin], one or more caseins [e.g., a κ-casein, a β-casein, a γ-casein, a mixture of a κ-casein and a β-casein, a mixture of a κ-casein and a γ-casein, a mixture of a β-casein and a γ-casein], or any combination thereof [i.e., a mixture of one or more whey proteins and one or more caseins]).

The optional milk protein component can comprise a native milk protein. The native milk protein can be a single native milk protein. The single native milk protein can be a single native whey protein (e.g., a native β-lactoglobulin, a native α-lactalbumin) or a single native casein (e.g., a native κ-casein, a native β-casein, a native γ-casein). Alternatively, the native milk protein can be two or more native milk proteins. The two or more native milk proteins can be two or more (e.g., two, three, four, five, six, seven, eight, nine, ten, or more) native whey proteins (e.g., a mixture of a native j3-lactoglobulin and a native α-lactalbumin, a mixture of two or more native β-lactoglobulins having different post-translational modifications (PTMs), a mixture of two or more native α-lactalbumins having different PTMs, a mixture of two or more native β-lactoglobulins having different PTMs and a native α-lactalbumin, a mixture of two or more native α-lactalbumins having different PTMs and a native β-lactoglobulin, a mixture of two or more native β-lactoglobulins having different PTMs and two or more native α-lactalbumins having different PTMs), two or more native caseins (e.g., a mixture of a native κ-casein and a native β-casein, a mixture of a native κ-casein and a native γ-casein, a mixture of a native β-casein and a native γ-casein, a mixture of two or more native κ-caseins having different PTMs, a mixture of two or more native β-caseins having different PTMs, a mixture of two or more native γ-caseins having different PTMs, a mixture of two or more native κ-caseins having different PTMs and a native β-casein, a mixture of two or more native κ-caseins having different PTMs and a native γ-casein, a mixture of two or more native β-caseins having different PTMs and a native κ-casein, a mixture of two or more native β-caseins having different PTMs and a native γ-casein, a mixture of two or more native γ-caseins having different PTMs and a native κ-casein, a mixture of two or more native γ-caseins having different PTMs and a native β-casein, a mixture of two or more native κ-caseins having different PTMs and/or two or more native β-caseins having different PTMs and/or two or more native γ-caseins having different PTMs), or any combination thereof (i.e., a mixture of one or more native whey proteins and one or more native caseins (e.g., one or both of a native β-lactoglobulin and a native α-lactalbumin in combination with one or two or all of a native κ-casein and a native β-casein and a native γ-casein)).

The optional milk protein component can comprise a recombinant milk protein. The recombinant milk protein can be a single recombinant milk protein. The single recombinant milk protein can be a single recombinant whey protein (e.g., a recombinant β-lactoglobulin, a recombinant α-lactalbumin) or a single recombinant casein (e.g., a recombinant κ-casein, a recombinant β-casein, a recombinant γ-casein). Alternatively, the recombinant milk protein can be two or more recombinant milk proteins. The two or more recombinant milk proteins can be two or more (e.g., two, three, four, five, six, seven, eight, nine, ten, or more) recombinant whey proteins (e.g., a mixture of a recombinant β-lactoglobulin and a recombinant α-lactalbumin, a mixture of two or more recombinant β-lactoglobulins having different post-translational modifications (PTMs), a mixture of two or more recombinant α-lactalbumins having different PTMs, a mixture of two or more recombinant β-lactoglobulins having different PTMs and a recombinant α-lactalbumin, a mixture of two or more recombinant α-lactalbumins having different PTMs and a recombinant β-lactoglobulin, a mixture of two or more recombinant β-lactoglobulins having different PTMs and two or more recombinant α-lactalbumins having different PTMs), two or more recombinant caseins (e.g., a mixture of a recombinant κ-casein and a recombinant β-casein, a mixture of a recombinant κ-casein and a recombinant γ-casein, a mixture of a recombinant β-casein and a recombinant γ-casein, a mixture of two or more recombinant κ-caseins having different PTMs, a mixture of two or more recombinant β-caseins having different PTMs, a mixture of two or more recombinant γ-caseins having different PTMs, a mixture of two or more recombinant κ-caseins having different PTMs and a recombinant β-casein, a mixture of two or more recombinant κ-caseins having different PTMs and a recombinant γ-casein, a mixture of two or more recombinant β-caseins having different PTMs and a recombinant κ-casein, a mixture of two or more recombinant β-caseins having different PTMs and a recombinant γ-casein, a mixture of two or more recombinant γ-caseins having different PTMs and a recombinant κ-casein, a mixture of two or more recombinant γ-caseins having different PTMs and a recombinant β-casein, a mixture of two or more recombinant κ-caseins having different PTMs and/or two or more recombinant β-caseins having different PTMs and/or two or more recombinant γ-caseins having different PTMs), or any combination thereof (i.e., a mixture of one or more recombinant whey proteins and one or more recombinant caseins (e.g., one or both of a recombinant β-lactoglobulin and a recombinant α-lactalbumin in combination with one or two or all of a recombinant κ-casein and a recombinant β-casein and a recombinant γ-casein)).

The optional milk protein component can consist of only a subset of whey proteins, or of a subset of caseins, or of a mixture of a subset of whey proteins and a subset of caseins (i.e., consists of some but not all proteins present in a whey protein concentrate, whey protein isolate, whey protein hydrolysate, casein isolate, casein concentrate, casein hydrolysate, milk protein isolate, milk protein concentrate, milk protein hydrolysate, micellar casein concentrate, sodium caseinate, acid caseinate). The subset of whey proteins can consist of a β-lactoglobulin and/or an α-lactalbumin. The subset of caseins can consist of a κ-casein and/or a β-casein and/or a γ-casein. The mixture of a subset of whey proteins and a subset of caseins can consist of a β-lactoglobulin and/or an α-lactalbumin in combination with a κ-casein and/or a β-casein and/or a γ-casein (e.g., a β-lactoglobulin and a κ-casein, an α-lactalbumin and a κ-casein, a β-lactoglobulin and an α-lactalbumin and a κ-casein).

A recombinant or native milk protein comprised in the optional milk protein component can be obtained from any mammalian species, including but not limited to cow, human, sheep, goat, buffalo, camel, horse, donkey, lemur, panda, guinea pig, squirrel, bear, macaque, gorilla, chimpanzee, mountain goat, monkey, ape, cat, dog, wallaby, rat, mouse, elephant, opossum, rabbit, whale, baboons, gibbons, orangutan, mandrill, pig, wolf, fox, lion, tiger, and echidna.

Methods for extracting native milk proteins and/or producing recombinant milk proteins are disclosed in U.S. Pat. No. 9,924,728, issued Mar. 27, 2018; U.S. publication US20190216106, published Jul. 18, 2019; and PCT publication WO2019213155, published Nov. 7, 2019; which are hereby incorporated herein in their entireties.

### Optional Non-Milk Protein Component

The optional non-milk protein component can comprise non-milk proteins obtained from any source, as well as mixtures of non-milk proteins obtained from various sources. Non-limiting examples of suitable sources include animals, plants, algae, fungi, and bacteria.

The non-milk protein component can comprise a recombinant non-milk protein. The recombinant non-milk protein can have a non-native PTM and/or lack an epitope that can elicit an immune response in a human or another animal.

### Optional Milk Fat Globule-Like Structure Component

The milk globule-like structure component can comprise milk globule-like structures having an average diameter of between 0.2 μm and 15 μm, 14 μm, 13 μm, 12 μm, 11 μm, 10 μm, 9 μm, 8 μm, 7 μm, 6 μm, 5 μm, 4 μm, 3 μm, 2 μm, 1 μm, or 0.5 μm; between 0.5 μm and 15 μm, 14 μm, 13 μm, 12 μm, 11 μm, 10 μm, 9 μm, 8 μm, 7 μm, 6 μm, 5 μm, 4 μm, 3 μm, 2 μm, or 1 μm; between 1 μm and 15 μm, 14 μm, 13 μm, 12 μm, 11 μm, 10 μm, 9 μm, 8 μm, 7 μm, 6 μm, 5 μm, 4 μm, 3 μm, or 2 μm; between 2 μm and 15 μm, 14 μm, 13 μm, 12 μm, 11 μm, 10 μm, 9 μm, 8 μm, 7 μm, 6 μm, 5 μm, 4 μm, or 3 μm; between 3 μm and 15 μm, 14 μm, 13 μm, 12 μm, 11 μm, 10 μm, 9 μm, 8 μm, 7 μm, 6 μm, 5 μm, or 4 μm; between 4 μm and 15 μm, 14 μm, 13 μm, 12 μm, 11 μm, 10 μm, 9 μm, 8 μm, 7 μm, 6 μm, or 5 μm; between 5 μm and 15 μm, 14 μm, 13 μm, 12 μm, 11 μm, 10 μm, 9 μm, 8 μm, 7 μm, or 6 μm; between 6 μm and 15 μm, 14 μm, 13 μm, 12 μm, 11 μm, 10 μm, 9 μm, 8 μm, or 7 μm; between 7 μm and 15 μm, 14 μm, 13 μm, 12 μm, 11 μm, 10 μm, 9 μm, or 8 μm; between 8 μm and 15 μm, 14 μm, 13 μm, 12 μm, 11 μm, 10 μm, or 9 μm; between 9 μm and 15 μm, 14 μm, 13 μm, 12 μm, 11 μm, or 10 μm; between 10 μm and 15 μm, 14 μm, 13 μm, 12 μm, or 11 μm; between 11 μm and 15 μm, 14 μm, 13 μm, or 12 μm; between 12 μm and 15 μm, 14 μm, or 13 μm; between 13 μm and 15 μm, or 14 μm; or between 14 μm and 15 μm.

The milk fat globule-like structures can comprise a milk lipid and a milk protein. The milk lipid can be a single milk lipid, or two or more distinct milk lipids. The milk protein can be a single milk protein, or two or more distinct milk proteins.

The milk globule-like structures can further comprise an other ingredient. Non-limiting examples of suitable other ingredients include glycoproteins, enzymes, water, cerebrosides, and any of the other ingredients disclosed herein.

### Optional Other Ingredients

Non-limiting examples of suitable other ingredients include bioactive agents, nutritional agents, and functional agents.

Non-limiting examples of bioactive agents include neutraceuticals (i.e., compounds that have physiological benefit or provide protection against chronic disease), and therapeutics (i.e., compounds that treat disease).

Non-limiting examples of therapeutics include clotting agents, anti-clotting agents, anti-inflammatory agents, neuroactive compounds, hormones, anti-microbial agents, enzymes, and antibodies.

Non-limiting examples of nutritional agents include nutritional supplements, prebiotics, probiotics, pro-vitamins, vitamins, minerals, antioxidants (i.e., molecules capable of slowing or preventing oxidation of other molecules), carbohydrates, and essential and semi-essential amino acids (e.g., cysteine, methionine, isoleucine, leucine, phenylanine, tryptophan, valine).

Non-limiting examples of vitamins include lipid soluble vitamins, water soluble vitamins, thiamin (vitamin B1), riboflavin (vitamin B2), niacin (vitamin B3), pantothenic acid (vitamin B5), vitamin B6 (pyridoxine), vitamin B12 (cobalamin), vitamin C, folate, vitamins A, vitamin D, vitamin E, vitamin K, and derivatives and mixtures thereof.

Non-limiting examples of minerals calcium, phosphorous, potassium, sodium, citrate, chloride, phosphate, magnesium, potassium, zinc, iron, molybdenum, manganese, copper, and mixtures thereof.

Non-limiting examples of antioxidants include α-tocopherol (e.g., tocopherol comprised in bovine milk), low molecular weight thiols (e.g., low molecular weight thiols comprised in bovine milk), retinol (e.g., retinol comprised in bovine milk), carotenoids (e.g., carotenoids comprised in cow milk, α-carotene, β-carotene, γ-carotene, lutein, zeaxanthin, astaxanthin), vitamin E, Azadirachta indica extract, riboflavin, rosemary extract, phenolic diterpenes (e.g., carnosol, carnosic acid) comprised in rosemary extract, sage extract, ascorbic acid (vitamin C) and its salts, lactic acid and its salts, grape residue silage, phenolic compounds (e.g., ferulic acid) comprised in grape residue silage, soybean (Glycine max) extract, isoflavones or polyphenolic compounds comprised in soybean extract, garlic (Allium sativum) extract, phenolic or flavonoid, or terpenoid compounds comprised in garlic extract, fennel (Foeniculum vulgare Mill.) extract, chamomile (Matricaria recutita L.) extract, fatty acids (e.g., alpha-lipoic acid), brown algae (e.g., Ascophyllum nodosum, Fucus vesiculosus), essential oils of green pink pepper (GEO), essential oils of mature pink pepper (MEO), green tea extract, butylated hydroxyanisole (E320), butylated hydroxytoluene (E321), polyphenols (e.g., curcumins, curcuminoids, desmethoxycurcumin (hydroxycirmamoyl feruloylmethane), bis-desmethoxycurcumin), catechins (e.g., epigallocatechin gallate, epicatechin gallate, epigallocatechin, epicatechin, C catechin, catechins comprised in green tea extract), and derivatives and mixtures thereof.

Non-limiting examples of carbohydrates include monosaccharides, disaccharides, and polysaccharides. Nonlimiting examples of monosaccharides include glucose, fructose, and dextrose. Non-limiting examples of disaccharides include maltose, lactose, and sucrose. Non-limiting examples of polysaccharides include maltodextrin, starches, flours, and edible fibers. Non-limiting examples of suitable starches include maltodextrin, inulin, fructooligosaccharides, pectin, carboxymethyl cellulose, guar gum, corn starch, oat starch, potato starch, rice starch, pea starch, and wheat starch. Non-limiting examples of suitable flours include but amaranth flour, oat flour, quinoa flour, rice flour, rye flour, sorghum flour, soy flour, wheat flour, and corn flour. Non-limiting examples of suitable edible fibers include bamboo fiber, barley bran, carrot fiber, citrus fiber, corn bran, soluble dietary fiber, insoluble dietary fiber, oat bran, pea fiber, rice bran, head husks, soy fiber, soy polysaccharide, wheat bran, wood pulp cellulose, and derivatives and mixtures thereof.

Non-limiting examples of functional agents include buffering agents, shelf life extending agents, pH and/or ionic strength adjusting agents (i.e., agents that raise or lower the pH and/or the ionic strength of a solution), preservatives, emulsifiers, plasticizers, texturing/mouthfeel agents coloring agents, flavor/aroma agents, and sweetening agents.

Non-limiting examples of shelf life extending agents include carbon monoxide, nitrites, sodium metabisulfite, Bombal, and derivatives and mixtures thereof.

Non-limiting examples of preservatives include p-hydroxybenzoate derivatives, sorbic acid, benzoic acid, nisin, natamycin, and derivatives and mixtures thereof.

Non-limiting examples of emulsifiers include include anionic emulsifiers, non-ionic emulsifiers, cationic emulsifiers, amphoteric emulsifiers, bioemulsifiers, steric emulsifiers, Pickering emulsifiers, glycolipids (e.g., trehalose lipids, sophorolipids, rhamnolipids, mannosylerythriol lipids), oligopeptides (e.g., gramicidin S, polymyxin), lipopeptides (e.g., surfactin), phospholipids, fatty acids, neutral lipids, polymeric biosurfactants, amphipathic polysaccharides, lipopolysaccharides, proteins (e.g., pea protein, soy protein, chickpea protein, algae protein, yeast protein, potato protein, lentil protein), mannoprotein, sodium phosphates, calcium stearoyl lactylate, mono- and diacetyl tartaric acid esters of monoglycerides, phospholipids, sorbitan monostearate, magnesium stearate, sodium/potassium/calcium salts of fatty acids, calcium stearoyl di lactate, poly-glycerol esters, sorbitan fatty acid esters, acetic acid esters of monoglycerides, lactic acid esters of monoglycerides, citric acid esters of monoglycerides, polyglycerol esters of fatty acids, polyglycerol polyricinoleate, propane-1,2-diol esters of fatty acids, sugar esters, sucrose esters of fatty acids, monoglycerides, acetylated monoglycerides, lactylated monoglycerides, diglycerides, phosphate monoglycerides, diacetyl tartaric acid esters, sodium/calcium stearoyl-2-lactylate, ammonium phosphatide, polysorbates, polysorbate-80, carboxymethylcellulose (CMC), modulated cellulose, citric acid esters, locust bean gum, guar gum, liposan, emulsan, lecithins (e.g., sunflower lecithin), surfactants (e.g., sorbitan trioleate (Span 85), sorbitan tristearate (Span 65), sorbitan sesquioleate (Arlacel 83), glyceryl monostearate, sorbitan monooleate (Span 80), sorbitan monostearate (Span 60), sorbitan monopalmitate (Span 40), sorbitan monolaurate (Span 20), polyoxyethylene sorbitan tristearate (Tween 65), polyoxyethylene sorbitan trioleate (Tween 85), polyethylene glycol 400 monostearate, polysorbate 60 (Tween 60), polyoxyethylene monostearate, polysorbate 80 (Tween 80), polysorbate 40 (Tween 40), polysorbate 20 (Tween 20), PEG 20 tristearate, PEG 20 trioleate, PEG 20 monostearate, PEG 20 monooleate, PEG 20 monopalmitate, and PEG 20 monolaurate sorbitan), and derivatives and mixtures thereof.

Non-limiting examples of plasticizers include diethanolamin, triethanolamine, glycerol, sorbitol, PEG-300, PEG-600, urea, octanoic acid, palmitic acid, dibutyl tartrate and phthalate, mono-, di-, or triglycerids esters, fructose, caproic acid, hydrocaproic acid, di-, tri-, or tetra-ethylene glycol, glycerol, 1,3-propane diol, 1,4-butane diol, 1,5-pentane diol, sucrose, and derivatives and mixtures thereof.

Non-limiting examples of texturing/mouthfeel agents include gums (e.g., guar gum, carob gum, wheat gum, xanthan gum), bulking agents, fillers, anti-adherent compounds, dispersing agents, moisture absorbing compounds, chemesthetic agents, film-forming agents, thickening agents, hardening agents, softening agents, stabilizers, anti-caking agents, anti-foaming agents, and derivatives and mixtures thereof.

Non-limiting examples of flavor/aroma agents include ethyl butyrate, 2-furyl methyl ketone, 2,3-pentanedione, γ-undecalactone, 6-undecalactone, propylene glycol, glycerol, ethyl alcohol, dimethylsulfide, 2-methylbutanol, 4-cis-heptenal 2-trans-nonenal, acetone, 2-undecanone, 2-butanone, amyl alcohol, 6-decalactone, 2-heptanone, 6-dodecalactone, 2-nonanone, 6-tetradecalactone, hydrogen sulfide, dimethyl sulfone, benzothiazole, 2-pentanone, 2-tridecanone, 6-octalactone, 2-pentadecanone, natural favors, artificial flavors (e.g., chocolate flavoring, coffee flavoring, strawberry flavoring, almond flavoring, hazelnut flavoring, vanilla flavoring, green tea flavoring, Irish cream flavoring, coconut flavoring), and derivatives and mixtures thereof.

The flavor/aroma agent can be a milk volatile organic compound (i.e., a volatile organic compound comprised in milk) that confers a milk or dairy flavor/aroma. Milk volatile organic compounds can be obtained, for example, by chemical synthesis, or by chemical or enzymatic degradation of milk lipids (e.g., by lipase-catalyzed hydrolysis of mTAGs, mDAGs, mMAGs, or mPLs to release mFFAs, which can directly contribute to flavor or act as precursors for production of other flavor compounds). Non-limiting examples of milk volatile organic compounds include lactones (e.g., 6-decalactone, 6-dodecalactone, 6-tetradecalactone, γ-decalactone, 6-octalactone), methyl ketones (e.g., acetone, 2-undecanone, 2-butanone, 2-heptanone, 2-nonanone, 2-pentanone, 2-tridecanone, 2-pentadecanone, acetoin), aldehydes, esters, alcohols (e.g., amyl alcohol), hydrocarbons, aromatic compounds, indole, methyl indole, phenolic compounds, dimethyl sulfide, hydrogen sulfide, dimethyl sulfone, benzothiazole, and diethylphthalate.

The flavor/aroma agent can be a green leaf volatile organic compound (i.e., a volatile organic compound comprised in green leaves) that confers a grassy flavor/aroma (see, for example, Gigot et al. 2010 Biotechnol Agron Soc Environ 14:451-460). Green leaf volatile organic compounds can be obtained, for example, by chemical synthesis, or by chemical and/or enzymatic degradation of fatty acids such as linoleic acid and linolenic acid. Linoleic acid and linolenic acid can be obtained, for example, from lipase treatment of natural oils (e.g., soy bean oil). Chemical and/or enzymatic degradation of linoleic acid and linolenic acid can involve, for example, hydroperoxidation (e.g., using a lipoxygenase [e.g., soy LOX (e.g., lipoxydase Type I-B (L7395, Sigma-Aldrich), Lipoxydase Type V (L6632, Sigma-Aldrich), potato LOX]; see, for example, Fuller et al. 2001 Arch Biochem Biophys 388:146-154) followed by acid-catalyzed cleavage or enzyme-catalyzed cleavage (e.g., using a hydroperoxide lyase), and optional conversion of short-chain aldehydes obtained to alcohols (e.g., using alcohol dehydrogenase). Non-limiting examples of green leaf volatile organic compounds include aldehydes having a carbon atom number that ranges from 6 to 12, and alcohols having a carbon atom number that ranges from 6 to 12. Non-limiting examples of such aldehydes and alcohols include hexanal, (Z)-3-hexenyl acetate, (Z)-3-hexenal, (Z)-3-hexenol, (Z)-2-hexenol, (E)-3-hexenol, (E)-2-hexenol, (E)-2-hexenal, (Z)-3-nonenol, €-2-nonenol, and 2,4-decadienal.

Non-limiting examples of sweetening agents include stevia, aspartame, cyclamate, saccharin, sucralose, mogrosides, brazzein, curculin, erythritol, glycyrrhizin, inulin, isomalt, lacititol, mabinlin, malititol, mannitol, miraculin, monatin, monelin, osladin, pentadin, sorbitol, thaumatin, xylitol, acesulfame potassium, advantame, alitame, aspartame-acesulfame, sodium cyclamate, dulcin, glucin, neohesperidin dihyrdochalcone, neotame, P-4000, honey, sucrose, corn syrup solids, glucose, lactose, galactose, fructose, maltose, isomaltulose, trehalose, maltodextrin, asulfame K, cyclamates, L-aspartyl-L-phenylalanine, tagatose, stevioside, hydrogenated starch hydrolysates, high-fructose corn syrup, fructooligosaccharides, polydextrose, and derivatives and mixtures thereof.

The other ingredient can be a lipid-soluble molecule that is not comprised in the milk lipid component or the optional non-milk lipid component. The lipid-soluble molecule can be a native molecule (i.e., a molecule extracted from nature) or a recombinant molecule (i.e., a molecule that is produced recombinantly). Non-limiting examples of suitable lipid-soluble molecules include micronutrients (e.g., carotenoids [e.g., beta-carotene]), vitamins (e.g., vitamin E, vitamin A, vitamin D, vitamin K), and lipid-soluble aroma compounds. The lipid-soluble molecule can be a recombinant beta-carotene. The lipid-soluble molecule can be a molecule that is produced by a recombinant host cell used in the production of a lipid comprised in the lipid component, or in the production of another component comprised in the composition provided herein.

### Imparted Attribute

The desirable attribute can be a physical attribute, chemical/biological attribute, sensory attribute, functional attribute, and any combination thereof.

Non-limiting examples of suitable physical attributes include appearance, color, translucence, opaqueness, shape, shape retention, structure, crystallinity, layering, aeration, solid content, hardness, softness, cohesion, plasticity, viscosity, density, and melting profile.

Non-limiting examples of suitable chemical/biological attributes include nutrient content (e.g., types and/or amounts of lipids, types and/or amounts of minerals, types and/or amounts of vitamins), pH, digestibility, oxidation stability, and hunger and/or satiety regulation.

Non-limiting examples of suitable sensory attributes include flavor, aroma, mouthfeel, fattiness, creaminess, richness, smoothness, and thickness.

Non-limiting examples of suitable functional attributes include gelling behavior (e.g., gelling capacity (i.e., capacity to form a gel (i.e., a protein network with spaces filled with solvent linked by hydrogen bonds to the protein molecules) having defined viscoelastic properties, as measured, for example, by the storage and elastic moduli and phase angle obtained in frequency sweeps on a rheometer) or by resistance to a physical and/or chemical condition (e.g., agitation, temperature, pH, ionic strength, protein concentration, sugar concentration, ionic strength)), gelling capacity over time (i.e., curve of gelling capacity over time), gel strength (i.e., mechanical force required to break a gel surface of a defined area, as measured, for example, by the storage modulus obtained in frequency sweeps on a rheometer), water holding capacity upon gelling, syneresis upon gelling (i.e., water weeping over time)); aggregation behavior (e.g., aggregation capacity (i.e., capacity to form a precipitate (i.e., a tight protein network based on strong interactions between protein molecules and exclusion of solvent), as measured, for example, by resistance to a physical and/or chemical condition), aggregation capacity over time (i.e., curvy of aggregation capacity over time)); foaming behavior (e.g., foaming capacity (i.e., capacity to form a foam, as measured, for example, by overrun and/or air phase volume), foam strength (i.e., measured, for example, as yield stress under shear or the amount of stress required to initiate flow in the sample), foam stability (i.e., half-life of foam in response to a physical and/or chemical condition), foam seep); thickening capacity; crystallization; lubricity; spreadability; and use versatility (i.e., ability to use the composition in a variety of manners and/or to derive a diversity of other compositions from the composition; e.g., ability to produce food products that resemble milk derivative products [e.g., any of the milk derivative products disclosed herein]).

### Composition

The composition and products prepared from same according to any of the above can be selected from the group consisting of cosmetic and personal care products (e.g., ointments, lotions, creams [e.g., moisturizing creams], cleansers, massage creams, soaps, hair shampoos, hair conditioners, skin masks, finishing products, hair tonics, toothpastes, chewing gums, gum-cleaning agents, skin lotions/creams), pharmaceutical products (e.g., products used for delivery of medicinal agents [e.g., micro- or nano-particles (e.g., beads, micelles) that encapsulate a therapeutic or nutraceutical for delivery (e.g., controlled delivery)], coatings of tablets, capsules, compacts, hydrogels), polymers (i.e., molecules composed of repeated molecular units that are covalently linked, either directly with each other or via intermediary molecules), compositions with industrial utility (e.g., dielectrics), and food products.

### Food Product

The composition and products prepared from same according to any of the above can be a food product.

The food product according to any of the above can be a conventional food product or resemble a conventional food product (i.e., can be a “substitute food product” that can be consumed or used in place of the conventional food product) selected from any of the food product categories defined by the National Health and Nutrition Examination Survey (NHANES), including, for example, snack foods and gums (e.g., snack bars, crackers, salty snacks from grain products, chewing gums); breads, grains, and pastas (e.g., oat breads and rolls, cornbread, corn muffins, tortillas, flour and dry mixes, biscuits, multi-grain breads and rolls, whole wheat breads and rolls, pastas, rye breads and rolls, cracked wheat breads and rolls, white breads and rolls); beverages (e.g., beers and ales, beverage concentrates, beverages, energy drinks, sports drinks, fluid replacements, soft drinks, carbonated beverages, juices, wine, cocktails, nutrition drinks, nutrition powders, protein-enriched beverages, coffee, tea, beer); sweets and desserts (e.g., cakes, candies, chips, cookies, cobblers, pastries, ices or popsicles, muffins, pies, sugar replacements or substitutes, syrups, honey, jellies, jams, preserves, salads, crepes, Danish, breakfast pastries, doughnuts); breakfast foods (e.g., cereal grains, cereal, rice, French toast, pancakes, waffles, coffee cake); eggs (e.g., whole egg (e.g., liquid whole egg, spray-dried whole egg, frozen whole egg), egg white (e.g., liquid egg white, spray-dried egg white, frozen egg white), egg dishes, egg soups, mixtures made with egg whites, egg substitutes, mixtures made with egg substitutes); salad dressings, oils, sauces, condiments (e.g., cooking fats, vegetable oils, salad dressings, tomato sauces, gravies); potatoes (e.g., potato salad, potato soups, chips and sticks, fried potatoes, mashed potatoes, stuffed potatoes, puffs); and soups (e.g., vegetable soups, vegetable broths), meals, main dishes, proteins (e.g., meat substitutes), and seafoods.

The food product can be an egg or egg product, or can resemble an egg or egg product (i.e., is an egg or egg product substitute). Non-limiting examples of suitable eggs and egg products include whole egg (e.g., liquid whole egg, spray-dried whole egg, frozen whole egg), egg white (e.g., liquid egg white, spray-dried egg white, frozen egg white), egg dishes, egg soups, and mixtures made with egg whites.

The food product can be a milk or dairy product, or can resemble a milk or dairy product (i.e., is a milk substitute or dairy product substitute). Non-limiting examples of milk and dairy products include milk (e.g., whole milk [at least 3.25% milk fat], partly skimmed milk [from 1% to 2% milk fat], skim milk [less than 0.2% milk fat], cooking milk, condensed milk, flavored milk, goat milk, sheep milk, dried milk, evaporated milk, milk foam), and products obtained from milk, including but not limited to yogurt (e.g., whole milk yogurt [at least 6 grams of fat per 170 g], low-fat yogurt [between 2 and 5 grams of fat per 170 g], nonfat yogurt [0.5 grams or less of fat per 170 g], greek yogurt [strained yogurt with whey removed], whipped yogurt, goat milk yogurt, Labneh [labne], sheep milk yogurt, yogurt drinks [e.g., whole milk Kefir, low-fat milk Kefir], Lassi), cheese (e.g., whey cheese such as ricotta; pasta filata cheese such as mozzarella; semi-soft cheese such as Havarti and Muenster; medium-hard cheese such as Swiss and Jarlsberg; hard cheese such as Cheddar and Parmesan; washed curd cheese such as Colby and Monterey Jack; soft ripened cheese such as Brie and Camembert; fresh cheese such as cottage cheese, feta cheese, cream cheese, and curd), processed cheese, processed cheese food, processed cheese product, processed cheese spread, enzyme-modulated cheese; cold-pack cheese), dairy-based sauces (e.g., salad dressing, bechamel sauce, fresh sauces, frozen sauces, refrigerated sauces, shelf stable sauces), dairy spreads (e.g., low-fat spread, low-fat butter), cream (e.g., dry cream, heavy cream, light cream, whipping cream, half-and-half, coffee whitener, coffee creamer, sour cream, creme fraiche), frozen confections (e.g., ice cream, smoothie, milk shake, frozen yogurt, sundae, gelato, custard), dairy desserts (e.g., fresh, refrigerated, or frozen), butter (e.g., whipped butter, cultured butter), dairy powders (e.g., whole milk powder, skim milk powder, fat-filled milk powder (i.e., milk powder comprising plant fat in place of all or some animal fat), infant formula, protein concentrate (i.e., protein content of at least 80% by weight; e.g., milk protein concentrate, whey protein concentrate, demineralized whey protein concentrate, β-lactoglobulin concentrate, α-lactalbumin concentrate, glycomacropeptide concentrate, casein concentrate), protein isolate (i.e., protein content of at least 90% by weight; e.g., milk protein isolate, whey protein isolate, demineralized whey protein isolate, β-lactoglobulin isolate, α-lactalbumin isolate, glycomacropeptide isolate, casein isolate), nutritional supplements, texturizing blends, flavoring blends, coloring blends), ready-to-drink or ready-to-mix products (e.g., fresh, refrigerated, or shelf stable dairy protein beverages, weight loss beverages, nutritional beverages, sports recovery beverages, and energy drinks), puddings, gels, chewables, crisps, bars (e.g., nutrition bars, protein bars), and fermented dairy products (e.g., yoghurt, cheese, sour cream, cultured buttermilk, cultured butter, cultured butter oil).

The food product can be an animal meat or an animal meat product, or can resemble an animal meat or animal meat product (i.e., is an animal meat substitute or animal meat product substitute). Non-limiting examples of animal meat include flesh obtained from skeletal muscle or from other organs (e.g., kidney, heart, liver, gallbladder, intestine, stomach, bone marrow, brain, thymus, lung, tongue), or parts thereof, obtained from an animal. The animal meat can be dark or white meat. Non-limiting examples of animals from which animal meat can be obtained include cattle, lamb, mutton, horse, poultry (e.g., chicken, duck, goose, turkey), fowl (e.g., pigeon, dove, grouse, partridge, ostrich, emu, pheasant, quail), fresh or salt water fish (e.g., catfish, tuna, spearfish, shark, halibut, sturgeon, salmon, bass, muskie, pike, bowfin, gar, eel, paddlefish, bream, carp, trout, walleye, snakehead, crappie, sister, mussel, scallop, abalone, squid, octopus, sea urchin, cuttlefish, tunicate), crustacean (e.g., crab, lobster, shrimp, barnacle), game animal (e.g., deer, fox, wild pig, elk, moose, reindeer, caribou, antelope, zebra, squirrel, marmot, rabbit, bear, beaver, muskrat, opossum, raccoon, armadillo, porcupine, bison, buffalo, boar, lynx, bobcat, bat), reptile (e.g., snakes, turtles, lizards, alligators, crocodiles), any insect or other arthropod, rodent (nutria, guinea pig, rat, mice, vole, groundhog, capybara), kangaroo, whale, and seal. The animal meat can be ground, chopped, shredded, or otherwise processed, and uncooked, cooking, or cooked.

Resemblance of the food product to a conventional food product can be due to any physical attribute, chemical/biological attribute, sensory attribute, functional attribute, and any combination thereof.

The food product can be or can resemble a dairy product, wherein the lipid component comprised in the food product imparts one or more attributes selected from the group consisting of creaminess, smoothness, flavor, aroma, mouthfeel, texture, palatability, reduced sensation of cold, melting point, and incorporation of air.

The food product can be or can resemble an animal meat product, wherein the lipid component comprised in the food product imparts one or more attributes selected from the group consisting of flavor, mouthfeel, texture, and/or aroma of an animal meat product.

The food product can be principally or entirely composed of components obtained from non-animal sources.

The food product can comprise between 5% and 100%, 90%, 80%, 70%, 60%, 50%, 40%, 30%, 20%, or 10%; between 10% and 100%, 90%, 80%, 70%, 60%, 50%, 40%, 30%, or 20%; between 20% and 100%, 90%, 80%, 70%, 60%, 50%, 40%, or 30%; between 30% and 100%, 90%, 80%, 70%, 60%, 50%, or 40%; between 40% and 100%, 90%, 80%, 70%, 60%, or 50%; between 50% and 100%, 90%, 80%, 70%, or 60%; between 60% and 100%, 90%, 80%, or 70%; between 70% and 100%, 90%, or 80%; between 80% and 100%, or 90%; or between 90% and 100% by mass of components obtained from non-animal sources.

The food product can be vegan, halal, and/or kosher.

The food product can be essentially free of one or more animal lipids.

The food product can be essentially free of one or more plant lipids. The food product can be essentially free of palm oil.

The food product can be essentially free of cholesterol, or can comprise less than 2%, less than 1.5%, less than 1%, less than 0.5%, less than 0.1%, or less than 0.05% by mass of cholesterol.

The food product can be essentially free of trans fatty acids or fatty acids.

The food product can be essentially free of allergenic epitopes (e.g., see, for example, Simonetta et al. 2012 Allergenicity of Milk Proteins, Milk Protein, Dr. Walter Hurley (ed.), InTech.), or resemble a conventional food product and have a reduced allergenicity of up to 5%, 10%, 15%, 20%, 25%, 30%, 35%, 40%, 45%, 50%, 55%, 60%, 65%, 70%, 75%, 80%, 85%, 90%, 95%, or 99% compared to such conventional food product.

### Method of Producing Lipid Component

In another aspect, provided herein is a method for producing the lipid component according to any of the above, wherein the method comprises the step of obtaining one or more milk lipids.

Obtaining one or more milk lipids can be accomplished using any method, including any one of or any combination of the following methods: chemical synthesis of a milk lipid or milk lipid precursor; extraction of a milk lipid or milk lipid precursor from a mammal-produced milk or milk fat; production of a milk lipid or milk lipid precursor in a recombinant host cell (e.g., any of the recombinant host cells provided herein); production of a milk lipid or milk lipid precursor in a cell-free system; production of a milk lipid or milk lipid precursor via fermentation of biomass (see, for example, Agler et al. Environ Sci Technol. 2012; 46(18):10229-10238); chemical or enzymatic hydrolysis of a vegetable oil (e.g., using SO3H-functional Bronsted acidic ionic liquids, and involving extraction of TAGs comprising shorter-chain fatty acids [e.g., via size-dependent aggregation, saponification, salting out), and chemical or enzymatic modification of a milk lipid precursor.

Chemical synthesis or chemical or enzymatic modification can involve a milk lipid precursor that can be chemically synthesized, extracted from a source (e.g., any of the sources disclosed herein), produced in a recombinant host cell (e.g., any of the recombinant host cells provided herein), or produced in a cell-free system. Enzymatic modification can be carried out by an enzyme that is produced in a recombinant host cell, or by an enzyme that is extracted from a native source (e.g., any of the sources disclosed herein that are no recombinant).

Fatty acids produced by the method provided herein can have the following attributes: have a carbon atom number that ranges from 4 to 54; be saturated or mono-unsaturated; be linear (not cyclic or branched); or any combination thereof. Glycerolipids produced by the method provided herein can have fatty acids having the following attributes: have a carbon atom number that ranges from 4 to 54; be saturated or mono-unsaturated; be linear (not cyclic or branched); or any combination thereof.

The method can further comprise the step of obtaining one or more non-milk lipids. Obtaining one or more non-milk lipids can be accomplished using any method, including any one or any combination of the methods disclosed herein for obtaining one or more milk lipids.

The method can further comprise the step of combining the one or more milk lipids to obtain the milk lipid component according to any of the above.

The method can further comprise the step of combining the one or more non-milk lipids to obtain the non-milk lipid component disclosed herein.

The method can further comprise the step of combining the milk lipid component with the non-milk lipid component.

The method can further comprise the step of combining the one or more milk lipids and the one or more non-milk lipids.

### Chemical/Enzymatic Modification

Chemical or enzymatic modification can involve chemical or enzymatic hydrolysis (i.e., removal) of a fatty acid from a glycerol backbone (e.g., a glycerol backbone of a MAG, DAG, TAG, or PL). Such hydrolysis can be accomplished by an enzyme (e.g., a lipase) that has selectivity for fatty acids of specific saturation level (i.e., saturated, mono-unsaturated, poly-unsaturated), carbon atom number (e.g., carbon atom number that ranges from 4 to 54, or larger than 54), or position within a glycerol backbone (e.g., sn1 position, sn2 position, sn3 position, sn1 and sn2 positions, sn1 and sn3 positions, sn2 and sn3 positions). Such hydrolysis can also be accomplished by an enzyme (e.g., a lipase) that has no specificity (i.e., acts randomly).

Chemical or enzymatic modification can involve chemical or enzymatic esterification (i.e., attaching) of a fatty acid to glycerol or a glycerol backbone (e.g., a glycerol backbone of a MAG or DAG). Such esterification can be accomplished by an enzyme (e.g., a diglyceride acyltransferase) that has selectivity for fatty acids of specific saturation level (i.e., saturated, mono-unsaturated, poly-unsaturated), carbon atom number (e.g., carbon atom number that ranges from 4 to 54, or larger than 54), or position within a glycerol backbone (e.g., sn1 position, sn2 position, sn3 position, sn1 and sn2 positions, sn1 and sn3 positions, sn2 and sn3 positions). Such esterification can also be accomplished by an enzyme (e.g., a lipase) that has no specificity (i.e., acts randomly).

Chemical or enzymatic modification can involve chemical or enzymatic hydrogenation (i.e., saturation) of an unsaturated carbon-carbon bond in a fatty acid or fatty acid. Such saturation can be accomplished by an enzyme (e.g., a saturase) that has selectivity for fatty acids or fatty acids having an unsaturated carbon-carbon bond at a specific location, and/or having a specific carbon atom number (e.g., carbon atom number that ranges from 4 to 54, or larger than 54) or position within a glycerol backbone (e.g., sn1 position, sn2 position, sn3 position, sn1 and sn2 positions, sn1 and sn3 positions, sn2 and sn3 positions). Such saturation can also be accomplished by an enzyme (e.g., a saturase) that has no specificity (i.e., acts randomly).

Chemical or enzymatic modification can involve chemical or enzymatic inter-esterification (i.e., reaction of a fatty acid ester [e.g., a fatty acid ester of a TAG, DAG, MAG, or PL] with FFAs [acidolysis], alcohols [alcoholysis], or with other fatty acid esters [trans-esterification] that can result in replacement of the fatty acid ester with a different fatty acid ester [e.g., exchange of a fatty acid on a given TAG with a different length fatty acid]). Such inter-esterification can involve an enzyme (e.g., a lipase) that has selectivity for fatty acids of specific saturation level (i.e., saturated, mono-unsaturated, poly-unsaturated), carbon atom number (e.g., carbon atom number that ranges from 4 to 54, or larger than 54), and/or position within a glycerol backbone (e.g., sn1 position, sn2 position, sn3 position, sn1 and sn2 positions, sn1 and sn3 positions, sn2 and sn3 positions). Inter-esterification can also involve an enzyme (e.g., a lipase) that has no specificity (i.e., acts randomly). As substrates for inter-esterification can serve FFAs, alcohols, or other fatty acid esters that comprise an alkyl group having a carbon atom number that ranges between 4 and 24. Suitable lipases for use in enzymatic inter-esterification can be extracted from natural sources (e.g., from microbial cells such as Miucor miehei, Rhizopus oryzae, Candida Antarctica, Pseudomonas cepacian, lactic acid bacteria, and fungal cells [e.g., yeast, filamentous fungal cells, mold]) or can be produced recombinantly (see, for example, Akoh et al. 2004 Lipids 39: 513-26. Yang et al. 2007 J Mol Catal B Enzym 45: 91-6; Kato et al. 2007 Appl Microbiol Biotechnol 2007; 75: 549-55).

### Production in Recombinant Host Cell

In another aspect, provided herein is a method for producing a milk lipid or milk lipid precursor in a recombinant host cell, wherein the method comprises the step of culturing a recombinant host cell provided herein under conditions suitable for producing the milk lipid or milk lipid precursor.

The method can further comprise the steps of:
a) obtaining the recombinant host cell;
b) purifying the milk lipid; and/or
c) post-processing the milk lipid.

### Recombinant Host Cell

In another aspect, provided herein is a recombinant host cell that is capable of producing a milk lipid (e.g., any of the milk lipids disclosed herein) or a milk lipid precursor, wherein the recombinant host cell comprises a genetic modification that essentially eliminates or modulates production and/or activity of a lipid biosynthesis-related protein compared to its parent cell (i.e., a cell that is identical to the recombinant host cell except that it does not comprise the genetic modification).

The lipid biosynthesis-related protein can be selected from the group consisting of
a) enzymes with activity in the production of an unsaturated fatty acid (e.g., any one enzyme disclosed herein that is active in the production of an unsaturated fatty acid, or any combination of two or more such enzymes);
b) enzymes with activity in the production of a fatty acid having a carbon atom number of greater than 16 (e.g., any one enzyme disclosed herein that is active in the production of a fatty acid having a carbon atom number of greater than 16, or any combination of two or more such enzymes);
c) enzymes with activity in the production of a fatty acid having a carbon atom number of 16 or less (e.g., any one enzyme disclosed herein that is active in the production of a fatty acid having a carbon atom number of 16 or less, or any combination of two or more such enzymes);
d) enzymes with activity in the β-oxidation pathway or peroxisome biogenesis (e.g., any one enzyme disclosed herein that is active in the β-oxidation pathway or peroxisome biogenesis, or any combination of two or more such enzymes);
e) enzymes with activity in the production of cytosolic acetyl-CoA (e.g., any one enzyme disclosed herein that is active in the production of cytosolic acetyl-CoA, or any combination of two or more such enzymes);
f) enzymes with activity in the production of a TAG, DAG, MAG, and/or PL (e.g., any one enzyme disclosed herein that is active in the production of a TAG, DAG, MAG, and/or PL, or any combination of two or more such enzymes);
g) enzymes with activity in the production of an amino acid (e.g., any one enzyme disclosed herein that is active in the overall production of an amino acid, or any combination of two or more such enzymes);
h) enzymes with activity in the production of cytosolic NADPH (e.g., any one enzyme disclosed herein that is active in the production of cytosolic NADPH, or any combination of two or more such enzymes);
i) enzymes with activity in inter-esterification (e.g., any one enzyme disclosed herein that is active in inter-esterification, or any combination of two or more such enzymes); and
j) combinations thereof.

Non-limiting examples of suitable enzymes active in the production of an unsaturated fatty acid include desaturases (e.g., A9 desaturase [Enzyme Commission (EC) #1.14.19.1], A12 desaturase [EC #1.14.19]). For example, the recombinant host cell according to any of the above can comprise a reduced or essentially eliminated production and/or activity of one or more enzymes with activity in the production of an unsaturated fatty acid.

Non-limiting examples of suitable enzymes with activity in the production of a fatty acid having a carbon atom number of greater than 16 include elongases (EC #2.3.1.199). For example, the recombinant host cell according to any of the above can comprise a reduced or essentially eliminated production and/or activity of one or more enzymes with activity in the production of a fatty acid having a carbon atom number of greater than 16.

Non-limiting examples of suitable enzymes with activity in the production of a fatty acid having a carbon atom number of 16 or less include enzymes active in the production of butyryl-ACP or butyryl-CoA (e.g., enzymes active in the acetyl-CoA-dependent synthesis of butyryl-CoA [e.g., acetyl-CoA carboxylase (EC #6.4.1.2), acetoacetyl-CoA thiolase (EC #2.3.1.9), acetoacetyl-CoA synthase (EC #2.3.1.194), ketoacyl-CoA thiolase (EC #2.3.1.16), crotonyl-CoA reductase (EC #1.3.1.86), hydroxyacyl-CoA dehydratase (EC #4.2.1.107), 3-hydroxybutyryl-CoA dehydrogenase (EC #1.1.1.157), butyryl-CoA transferase], enzymes active in the ACP-dependent synthesis of butyryl-ACP [e.g., malonyl-CoA:ACP transacylase (EC #2.3.1.39), acetyl-CoA:ACP transacylase (EC #2.3.1.38), beta-ketoacyl-ACP synthetase (EC #2.3.1.41), 3-oxoacyl-ACP reductase (EC #1.1.1.100, EC #1.1.1.112), 3-hydroxyacyl-ACP dehydratase (EC #4.2.1.59), enoyl-ACP reductase (EC #1.3.1.10, EC #1.3.1.9)]), ACP thioesterases with specificity for shorter chain fatty acids (e.g., ACP thioesterases of Anaerococcus tetradius, Cuphea hookeriana, Cuphea palustris, Clostridium perfringens, and Umbellularia californica [see, for example, Rutter et al. 2015 Appl Microbiol Biotechnol 99:7359-7368]; ACP thioesterases of Escherchia co/i [e.g., YciA, tesB, YbgC, YbfF]), and fatty acid synthases that produce shorter-chain fatty acids (e.g., hexanoate synthase [see, for example, Hitchman et al. 2001. Bioorg Chem 29:293-307], octanoate synthase [see, for example, Culceth et al. 1998 Tetrahedron Lett. 39:1949-1952]), fatty acid synthases produced by cells of the mammary gland of a mammal (e.g., FASN [UniProt #Q71SP7, and homologs and orthologs thereof]), and fatty acid synthases comprising one or more amino acid substitutions, deletions, and/or additions, and/or domain replacements, that modify the catalytic activity of the synthases such that they produce an increased level of fatty acids having a carbon atom number of between 4 and 16 (e.g., fungal [e.g., Saccharomyces cerevisiae] FAS1 comprising an I306A substitution and/or fungal [e.g., Saccharomyces cerevisiae] FAS2 comprising a R1834K, G1250S, and/or M1251W substitution [see, for example, Gajewski et al. 2017 Nature Commun 8:14650], fungal [e.g., Yarrowia lipolytica] FAS1 comprising a thioesterase with broad range of chain specificity [e.g., TesA or Ybgec of Escherichia coli] in place of its MPT transferase domain [see, for example, Xu et al. 2016 PNAS 113:10848-10853]). The recombinant host cell according to any of the above can comprise an increased production and/or activity of one or more such enzymes. The recombinant host cell according to any of the above can comprise an increased production and/or activity of one or more enzymes active in the malonyl-ACP-dependent synthesis of butyryl-ACP and a decreased or essentially eliminated production and/or activity of one or more enzymes active in the malonyl-CoA-dependent synthesis of butyryl-CoA. The recombinant host cell according to any of the above can comprise an increased production and/or activity of one or more enzymes active in the malonyl-CoA-dependent synthesis of butyryl-CoA and a decreased or essentially eliminated production and/or activity of one or more enzymes active in the ACP-dependent synthesis of butyryl-ACP (see, for example, U.S. patent publication US20160340700, published Nov. 24, 2016).

Non-limiting examples of enzymes active in β-oxidation or peroxisome biogenesis include acyl-CoA oxidases (EC #1.3.3.6), MFE1 (EC #4.2.1.74), PEX1 (UniProt #s Q9UV06 and P24004, and homologs and orthologs thereof), PEX2 (UniProt #s P32800, Q99155, and homologs and orthologs thereof), PEX3 (UniProts #Q874C0 and P28795, and homologs and orthologs thereof), PEX4 (UniProt #s Q9FMA3 and Q99144, and homologs and orthologs thereof), PEX5 (UniProt #s Q99144 and P35056, and homologs and orthologs thereof), PEX6 (UniProt #s P33760 and P36966, and homologs and orthologs thereof), and PEX10 (UniProt #s Q05568 and Q9P4U5, and homologs and orthologs thereof). For example, the recombinant host cell according to any of the above can comprise a decreased or essentially eliminated production and/or activity of one or more enzymes that are active β-oxidation or peroxisome biogenesis (see, for example, Luo et al., 2002 Arch Biochem Biophys 407:32-38; Blazeck et al. 2014 Nature Commun. 5:3131).

Non-limiting examples of suitable enzymes active in the production of cytosolic acetyl-CoA include pyruvate decarboxylase (EC #4.1.1.1; e.g., PDC1 and homologs and orthologs thereof]), aldehyde dehydrogenase (EC #1.2.1.4; e.g., ALD6 and homologs and orthologs thereof]), and acetyl-CoA carboxylase (EC #6.4.1.2; e.g., ACS1 and homologs and orthologs thereof]). For example, the recombinant host cell according to any of the above can comprise an increased production and/or activity of one or more enzymes that are active in the production of cytosolic acetyl-CoA (see, for example, Koivuranta et al. 2018. Front Microbiol 9:1337).

Non-limiting examples of suitable enzymes active in the production of a TAG, DAG, MAG, and/or PL include phospholipid:diacylglycerol acyltransferases (EC #2.3.1.158), acyl-CoA:diacylglycerol acyltransferases (EC #2.3.1.20), glycerol-3-phosphate acyltransferase (EC #s 2.3.1.n5, 2.3.1.198, 2.3.1.52, 2.3.1.51, 2.3.1.15), acylglycerophosphate acyltransferase (EC #2.3.1.n4), and phosphatidic acid phosphohydrolase (EC #3.1.3.4), dihydroxyacetone phosphate acyltransferase (EC #2.3.1.42), and 2-acylglycerol O-acyltransferase (EC #2.3.1.22). For example, the recombinant host cell according to any of the above can comprise an increased production and/or activity of one or more enzymes that are active in production of a TAG, DAG, MAG, and/or PL (see, for example, Koivuranta et al. 2018 Front Microbiol 9:1337; Tai & Stephanopoulos 2013 Metab Eng 15:1-9).

Non-limiting examples of suitable enzymes active in the production of an amino acid include 3-isopropylmalate dehydrogenase (EC #1.1.1.85) and orotidine-5′-phosphate decarboxylase (EC #4.1.1.23). For example, the recombinant host cell according to any of the above can comprise an increased production and/or activity of one or more enzymes that are active in production of an amino acid (see, for example, Blazeck et al. 2014 Nature Commun. 5:3131).

Non-limiting examples of enzymes active in the production of cytosolic NADPH are disclosed, for example, by Qiao et al. 2016 (Nat Biotechnol 35(2):173-177). For example, the recombinant host cell according to any of the above can comprise an increased production and/or activity of any one or more enzymes that are active in the production of cytosolic NADPH.

Non-limiting examples of enzymes active in inter-esterification include intracellular lipases (i.e., lipases that are comprised inside the recombinant host cell), extracellular lipases (i.e., lipases that are secreted by the recombinant host cell), lipases that catalyze FFA esterification, lipases that hydrolyze an ester bond in sn1 position of a TAG, lipases that hydrolyze an ester bond in sn2 position of a TAG, lipases that hydrolyze an ester bond in sn3 position of a TAG, lipases that hydrolyze ester bonds in sn1 and sn2 positions of a TAG, lipases that hydrolyze ester bonds in sn2 and sn3 positions of a TAG, lipases that hydrolyze ester bonds in sn1 and sn3 positions of a TAG, lipases that do not distinguish between positions of esters of a TAG, or any suitable lipase disclosed herein. For example, the recombinant host cell according to any of the above can comprise an increased production and/or activity of any one or more lipases that are active in inter-esterification.

The genetic modification comprised in the recombinant host cell can be a single genetic modification, or two or more genetic modifications.

The genetic modification comprised in the recombinant host cell can increase production of a lipid biosynthesis-related protein, decrease production of a lipid biosynthesis-related protein, increase activity of a lipid biosynthesis-related protein, decrease activity of a lipid biosynthesis-related protein, essentially eliminate production of a lipid biosynthesis-related protein, essentially eliminate activity of a lipid biosynthesis-related protein, or effect any combination of two or more of the above in comparison to production or activity of the lipid biosynthesis-related protein in its parent cell (i.e., a cell that is identical to the recombinant host cell except that it does not comprise the genetic modification).

The lipid biosynthesis-related protein of which production and/or activity is modulated or essentially eliminated in the recombinant host cell according to any of the above can be a single lipid biosynthesis-related protein (e.g., a single enzyme with activity in the production of an unsaturated fatty acid; a single enzyme with activity in the production of a fatty acid having a carbon atom number of greater than 16; a single enzyme with activity in the production of a fatty acid having a carbon atom number of 16 or less; a single enzyme with activity in the β-oxidation pathway or peroxisome biogenesis; a single enzyme with activity in the production of cytosolic acetyl-CoA; a single enzyme with activity in the production of a TAG, DAG, MAG, and/or PL; a single enzyme with activity in the production of an amino acid; a single enzyme with activity in the production of cytosolic NADPH; a single enzyme with activity in inter-esterification), or two or more lipid biosynthesis-related proteins (e.g., two or more enzymes with activity in the production of an unsaturated fatty acid; two or more enzymes with activity in the production of a fatty acid having a carbon atom number of greater than 16; two or more enzymes with activity in the production of a fatty acid having a carbon atom number of 16 or less; two or more enzymes with activity in the β-oxidation pathway or peroxisome biogenesis; two or more enzymes with activity in the production of cytosolic acetyl-CoA; two or more enzymes with activity in the production of a TAG, DAG, MAG, and/or PL; two or more enzymes with activity in the production of an amino acid; two or more enzymes with activity in the production of cytosolic NADPH; two or more enzymes with activity in inter-esterification; any combination of one or more enzymes with activity in the production of an unsaturated fatty acid; one or more enzymes with activity in the production of a fatty acid having a carbon atom number of greater than 16; one or more enzymes with activity in the production of a fatty acid having a carbon atom number of 16 or less; one or more enzymes with activity in the β-oxidation pathway or peroxisome biogenesis; one or more enzymes with activity in the production of cytosolic acetyl-CoA; one or more enzymes with activity in the production of a TAG, DAG, MAG, and/or PL; one or more enzymes with activity in the production of an amino acid; one or more enzymes with activity in the production of cytosolic NADPH; one or more enzymes with activity in inter-esterification).

### Obtaining Recombinant Host Cell

A recombinant host cell according to any of the above can be obtained by introducing into a parent cell a genetic modification.

The genetic modification can be any genetic modification that modulates or essentially eliminates production and/or activity of a lipid biosynthesis-related protein (e.g., any one of the lipid biosynthesis-related proteins disclosed herein or any combination of at least two lipid biosynthesis-related proteins disclosed herein). For example, the recombinant host cell of any of the above can comprise:
a) a genetic modification in a regulatory element, or a functional part thereof (i.e., a part that is sufficient for the function of the regulatory element), that drives expression of a lipid biosynthesis-related protein, wherein the genetic modification modulates or essentially eliminates expression of the lipid biosynthesis-related protein;
b) a genetic modification in a coding sequence that encodes a lipid biosynthesis-related protein, or a functional part thereof (e.g., a catalytic domain), wherein the genetic modification modulates or essentially eliminates activity of the lipid biosynthesis-related protein;
c) a genetic modification in a regulatory element, or a functional part thereof (i.e., a part that is sufficient for the function of the regulatory element), that drives expression of a protein required for expression of a lipid biosynthesis-related protein (e.g., a transcription factor, a post-translational modification enzyme required for production of an active form of a lipid biosynthesis-related protein), wherein the genetic modification modulates or essentially eliminates expression of the protein required for expression of the lipid biosynthesis-related protein and thereby modulates or essentially eliminates expression of the lipid biosynthesis-related protein;
d) a genetic modification in a coding sequence that encodes a protein required for expression of a lipid biosynthesis-related protein, or a functional part thereof (e.g., a DNA binding domain of a transcription factor, a catalytic domain of a post-translational modification enzyme), wherein the genetic modification modulates or essentially eliminates activity of the protein required for expression of the lipid biosynthesis-related protein and thereby modulates or essentially eliminates expression of the lipid biosynthesis-related protein;
e) a genetic modification in a regulatory element, or a functional part thereof (i.e., a part that is sufficient for the function of the regulatory element), that drives expression of an endogenous inhibitor of a lipid biosynthesis-related protein, wherein the genetic modification modulates or essentially eliminates expression of the endogenous inhibitor and thereby modulates or essentially eliminates expression of the lipid biosynthesis-related protein;
f) a genetic modification in a coding sequence that encodes an endogenous inhibitor of a lipid biosynthesis-related protein, wherein the genetic modification modulates or essentially eliminates activity of the endogenous inhibitor and thereby modulates or essentially eliminates activity of the lipid biosynthesis-related protein;
g) a genetic modification that introduces a coding sequence that encodes a heterologous (i.e., non-native) inhibitor of a lipid biosynthesis-related protein (e.g., an inhibitory molecule, a nucleotide sequence that is complementary to a coding sequence encoding a lipid biosynthesis-related protein, an RNAi construct that is specific to a lipid biosynthesis-related protein), wherein the genetic modification provides for production of the heterologous inhibitor and thereby modulates or essentially eliminates activity of the lipid biosynthesis-related protein;
h) a genetic modification that introduces a coding sequence that encodes a heterologous (i.e., non-native) activator of a lipid biosynthesis-related protein, wherein the genetic modification provides for production of the heterologous activator and thereby modulates activity of the lipid biosynthesis-related protein; and/or
i) a genetic modification that introduces a coding sequence that encodes a heterologous lipid biosynthesis-related protein and thereby modulates activity of the heterologous lipid biosynthesis-related protein.

A genetic modification can consist of, for example, an insertion, a substitution, a duplication, a rearrangement and/or a deletion of one or more nucleotides in a genome of a cell. A genetic modification can, for example, introduce a stop codon; remove a start codon; insert a frame-shift of the open reading frame; or create a point mutation, missense mutation, substitution mutation, deletion mutation, frameshift mutation, insertion mutation, duplication mutation, amplification mutation, translocation mutation, or inversion mutation.

The genetic modification can lead to any of the following in the recombinant host cell compared to its parent cell: increased production of one or more saturated fatty acids or of one or more glycerolipids comprising one or more saturated fatty acids; increased production of one or more FFAs (e.g., saturated FFAs, mono-unsaturated FFAs, poly-unsaturated FFAs) having a carbon atom number of between 4 and 24, or of one or more TAGs, DAGs, MAGs, and/or PLs comprising one or more fatty acids (e.g., saturated fatty acids, mono-unsaturated fatty acids, poly-unsaturaned fatty acids) having a carbon atom number of between 4 and 24; increased production of one or more fatty acids that are essentially free of rings or cyclic structures; increased production of one or more milk lipids; increased production of one or more milk lipid precursors; increased secretion of one or more FFAs (e.g., saturated FFAs, mono-unsaturated FFAs, poly-unsaturated FFAs) having a carbon atom number of between 4 and 24, or of one or more TAGs, DAGs, MAGs, and/or PLs comprising one or more fatty acids (e.g., saturated fatty acids, mono-unsaturated fatty acids, poly-unsaturaned fatty acids) having a carbon atom number of between 4 and 24; reduced or eliminated production of one or more unsaturated fatty acids or of one or more glycerolipids comprising one or more unsaturated fatty acids; reduced or eliminated production of one or more FFAs (e.g., saturated FFAs, mono-unsaturated FFAs, poly-unsaturated FFAs) having a carbon atom number that is greater than 10 (e.g., greater than 10, greater than 12, greater than 14, greater than 16, greater than 18, greater than 20, greater than 22, greater than 24, greater than 26, greater than 28, greater than 30, greater than 40, or greater than 50), or of one or more glycerolipids comprising one or more fatty acids (e.g., saturated fatty acids, mono-unsaturated fatty acids, poly-unsaturated fatty acids) having a carbon atom number that is greater than 10 (e.g., greater than 10, greater than 12, greater than 14, greater than 16, greater than 18, greater than 20, greater than 22, greater than 24, greater than 26, greater than 28, greater than 30, greater than 40, or greater than 50); reduced or eliminated secretion of one or more FFAs (e.g., saturated FFAs, mono-unsaturated FFAs, poly-unsaturated FFAs) having a carbon atom number that is greater than 10 (e.g., greater than 10, greater than 12, greater than 14, greater than 16, greater than 18, greater than 20, greater than 22, greater than 24, greater than 26, greater than 28, greater than 30, greater than 40, or greater than 50), or of one or more glycerolipids comprising one or more fatty acids (e.g., saturated fatty acids, mono-unsaturated fatty acids, poly-unsaturated fatty acids) having a carbon atom number that is greater than 10 (e.g., greater than 10, greater than 12, greater than 14, greater than 16, greater than 18, greater than 20, greater than 22, greater than 24, greater than 26, greater than 28, greater than 30, greater than 40, or greater than 50); or any combination thereof.

The recombinant host cell according to any of the above can be obtained by any method known in the art for modifying (e.g., increasing, decreasing, knocking out, knocking in) catalytic activity and/or catalytic specificity and/or production levels of endogenous or heterologous proteins. Non-limiting examples of such methods include targeted or random mutagenesis, adaptive experimental evolution (e.g., adaption to UV irradiation, oxidative stress, pH), and introduction of heterologous polynucleotides.

The one or more genetic modifications comprised in the recombinant host cell according to any of the above can be analyzed using any suitable method known in the art, such as assays that are carried out at the DNA (e.g., genomic DNA) level or RNA level. Non-limiting examples of such assays include Northern blotting, dot blotting (DNA or RNA), RT-PCR (reverse transcriptase polymerase chain reaction), in situ hybridization, and Southern blotting.

The recombinant host cell according to any of the above can comprise a modulated or essentially eliminated production and/or activity of a lipid biosynthesis-related protein (e.g., a lipid biosynthesis-related protein disclosed herein or any combination of two or more endogenous proteins disclosed herein) that is reduced by 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or 100%.

The recombinant host cell according to any of the above can comprise a modulated production and/or activity of a lipid biosynthesis-related protein (e.g., a lipid biosynthesis-related protein disclosed herein or any combination of two or more endogenous proteins disclosed herein) that is increased by 5% or more, 10% or more, 15% or more, 20% or more, 25% or more, 50% or more, 75% or more, 100% or more, 150% or more, 200% or more, 300% or more, 400% or more, 500% or more, 600% or more, 700% or more, 800% or more, 900% or more, or 1,000% or more.

The recombinant host cell according to any of the above can produce a milk lipid or a milk lipid precursor at a titer and/or productivity that is at least 1.1-fold or more, 1.2-fold or more, 1.3-fold or more, 1.4-fold or more, 1.5-fold, 1.6-fold or more, 1.7-fold or more, 1.8-fold or more, 1.9-fold or more, 2-fold or more, 3-fold or more, 4-fold or more, 5-fold or more, 6-fold or more, 7-fold or more, 8-fold or more, 9-fold or more, or 10-fold or more of that produced by its parent host cell (i.e., an identical host cell that does not comprise the genetic modification).

### Parent Cell

The parent cell can be obtained from any organism (e.g., animals, plants, microbes [e.g., fungi (e.g. yeast, filamentous fungi), bacteria, algae, archaea, protozoa]).

Non-limiting examples of suitable animals include insects (e.g., fly), mammals (e.g. cow, sheep, goat, rabbit, pig, human), and birds (e.g., chicken).

Non-limiting examples of suitable plants include cycad, Ginkgo biloba, conifer, cypress, juniper, thuja, cedarwood, pine, angelica, caraway, coriander, cumin, fennel, parsley, dill, dandelion, helichrysum, marigold, mugwort, safflower, camomile, lettuce, wormwood, calendula, citronella, sage, thyme, chia seed, mustard, olive, coffee, capsicum, eggplant, paprika, cranberry, kiwi, vegetables (e.g., carrot, celery), tagete, tansy, tarragon, sunflower, wintergreen, basil, hyssop, lavender, lemon verbena, marjoram, melissa, patchouli, pennyroyal, peppermint, rosemary, sesame, spearmint, primrose, samara, pepper, pimento, potato, sweet potato, tomato, blueberry, nightshade, petunia, morning glory, lilac, jasmin, honeysuckle, snapdragon, psyllium, wormseed, buckwheat, amaranth, chard, quinoa, spinach, rhubarb, jojoba, cypselea, chlorella, marula, hazelnut, canola, kale, bok choy, rutabaga, frankincense, myrrh, elemi, hemp, pumpkin, squash, curcurbit, manioc, dalbergia, legume plants (e.g., alfalfa, lentil, bean, clover, pea, fava coceira, frijole bola roja, frijole negro, lespedeza, licorice, lupin, mesquite, carob, soybean, peanut, tamarind, wisteria, cassia, chickpea/garbanzo, fenugreek, green pea, yellow pea, snow pea, lima bean, fava bean), geranium, flax, pomegranate, cotton, okra, neem, fig, mulberry, clove, eucalyptus, tea tree, niaouli, fruiting plant (e.g., apple, apricot, peach, plum, pear, nectarine), strawberry, blackberry, raspberry, cherry, prune, rose, tangerine, citrus (e.g., grapefruit, lemon, lime, orange, bitter orange, mandarin, tangerine), mango, citrus bergamot, buchu, grape, broccoli, brussels sprout, camelina, cauliflower, rape, rapeseed (canola), turnip, cabbage, cucumber, watermelon, honeydew melon, zucchini, birch, walnut, cassava, baobab, allspice, almond, breadfruit, sandalwood, macadamia, taro, tuberose, aloe vera, garlic, onion, shallot, vanilla, yucca, vetiver, galangal, barley, corn, Curcuma aromatica, ginger, lemon grass, oat, palm, pineapple, rice, rye, sorghum, triticale, turmeric, yam, bamboo, barley, cajuput, canna, cardamom, maize, oat, wheat, cinnamon, sassafras, Lindera benzoin, bay laurel, avocado, ylang-ylang, mace, nutmeg, moringa, horsetail, oregano, cilantro, chervil, chive, aggregate fruit, grain plant, herbal plant, leafy vegetable, non-grain legume plant, nut plant, succulent plant, land plant, water plant, delbergia, millet, drupe, schizocarp, flowering plant, non-flowering plant, cultured plant, wild plant, tree, shrub, flower, grass, herbaceous plant, brush, lianas, cacti, tropical plant, subtropical plant, temperate plant, moss (e.g., Physcomitrella patens), and derivatives and crosses thereof.

Non-limiting examples of suitable yeast include members of any of the following genera, and derivatives and crosses thereof. Candida (e.g., Candida albicans, Candida etchellsii, Candida guilliermondii, Candida humilis, Candida lipolytica, Candida orthopsilosis, Candida palmioleophila, Candida pseudotropicalis, Candida sp., Candida utilis, Candida versatilis), Cladosporium, Cryptococcus (e.g., Cryptococcus terricolus, Cryptococcus curvatus), Debaryomyces (e.g., Debaryomyces hansenii), Endomyces (e.g., Endomyces vernalis), Endomycopsis (e.g., Endomycopsis vernalis), Eremothecium (e.g., Eremothecium ashbyii), Hansenula (e.g., Hansenula sp., Hansenula polymorpha), Kluyveromyces (e.g., Kluyveromyces sp., Kluyveromyces lactis, Kluyveromyces marxianus var. lactis, Kluyveromyces marxianus, Kluyveromyces thermotolerans), Lipomyces (e.g., Lipomyces starkeyi, Lipomyecs lipofer), Ogataea (e.g., Ogataea minuta), Pichia (e.g., Pichia sp., Pichia pastoris, Pichia finlandica, Pichia trehalophila, Pichia koclamae, Pichia membranaefaciens, Pichia minuta, Pichia lindneri), Pichia opuntiae, Pichia thermotolerans, Pichia salictaria, Pichia guercuum, Pichia pijperi, Pichia stiptis, Pichia methanolica), Rhodosporidium (e.g., Rhodosporidium toruloides), Rhodotorula (e.g., Rhodotorula sp., Rhodotorula gracilis, Rhodotorula glutinis, Rhodotorula graminis), Saccharomyces (e.g., Saccharomyces sp., Saccharomyces bayanus, Saccharomyces beticus, Saccharomyces cerevisiae, Saccharomyces chevalieri, Saccharomyces diastaticus, Saccharomyces ellipsoideus, Saccharomyces exiguus, Saccharomyces florentinus, Saccharomyces fragilis, Saccharomyces pastorianus, Saccharomyces pombe, Saccharomyces sake, Saccharomyces uvarum), Sporobolomyces (e.g., Sporobolomyces roseus), Sporidiobolus (e.g., Sporidiobolus johnsonii, Sporidiobolus salmonicolor), Trichosporon (e.g., Trichosporon cacaoliposimilis, Trichosporon oleaginosus sp. nov., Trichosporon cacaoliposimilis sp. nov., Trichosporon gracile, Trichosporon dulcitum, Trichosporon jirovecii, Trichosporon insectorum, Trichosporon fermentans), Xanthophyllomyces (e.g., Xanthophyllomyces dendrorhous), Yarrowia (e.g., Yarrowia lipolytica), and Zygosaccharomyces (e.g., Zygosaccharomyces rouxii).

Non-limiting examples of suitable filamentous fungi include any holomorphic, teleomorphic, and anamorphic forms of fungi, including members of any of the following genera, and derivatives and crosses thereof. Acremonium (e.g., Acremonium alabamense), Aspergillus (e.g., Aspergillus aculeatus, Aspergillus awamori, Aspergillus clavatus, Aspergillus flavus, Aspergillus foetidus, Aspergillus fumigatus, Aspergillus japonicus, Aspergillus nidulans, Aspergillus niger, Aspergillus niger var. awamori, Aspergillus ochraceus, Aspergillus oryzae, Aspergillus sojae, Aspergillus terreus, as well as Emericella, Neosartorya, and Petromyces species), Aureobasidium, Canariomyces, Chaetomium, Chaetomidium, Corynascus, Chrysosporium (e.g., Chrysosporium botryoides, Chrysosporium carmichaeli, Chrysosporium crassitunicatum, Chrysosporium europae, Chrysosporium evolceannui, Chrysosporium farinicola, Chrysosporium fastidium, Chrysosporium filiforme, Chrysosporium georgiae, Chrysosporium globiferum, Chrysosporium globiferum var. articulatum, Chrysosporium globiferum var. niveum, Chrysosporium hirundo, Chrysosporium hispanicum, Chrysosporium holmii, Chrysosporium indicum, Chrysosporium iops, Chrysosporium keratinophilum, Chrysosporium kreiselii, Chrysosporium kuzurovianum, Chrysosporium lignorum, Chrysosporium obatum, Chrysosporium lucknowense, Chrysosporium lucknowense Garg 27K, Chrysosporium medium, Chrysosporium medium var. spissescens, Chrysosporium mephiticum, Chrysosporium merdarium, Chrysosporium merdarium var. roseum, Chrysosporium minor, Chrysosporium pannicola, Chrysosporium parvum, Chrysosporium parvum var. crescens, Chrysosporium pilosum, Chrysosporium pseudomerdarium, Chrysosporium pyriformis, Chrysosporium queenslandicum, Chrysosporium sigleri, Chrysosporium sulfureum, Chrysosporium synchronum, Chrysosporium tropicum, Chrysosporium undulatum, Chrysosporium vallenarense, Chrysosporium vespertilium, Chrysosporium zonatum), Coonemeria, Cunninghamella (e.g., Cunninghamella ehinulata), Dactylomyces, Emericella, Filibasidium, Fusarium (e.g., Fusarium moniliforme, Fusarium venenatum, Fusarium oxysporum, Fusarium graminearum, Fusarium proliferatum, Fusarium verticiollioides, Fusarium culmorum, Fusarium crookwellense, Fusarium poae, Fusarium sporotrichioides, Fusarium sambuccinum, Fusarium torulosum, as well as associated Gibberella teleomorphic forms thereof), Gibberella, Humicola, Hypocrea, Lentinula, Malbranchea (e.g., Malbranchea filamentosa), Magnaporthe, Malbranchium, Melanocarpus, Mortierella (e.g., Mortierella alpina 1S-4, Mortieralla isabelline, Mortierrla vinacea, Mortieralla vinaceae var. raffinoseutilizer), Mucor (e.g., Mucor miehei Cooney et Emerson (Rhizomucor miehei (Cooney & R. Emerson)) Schipper, Mucor pusillus Lindt, Mucor circinelloides Mucor mucedo), Myceliophthora (e.g., Myceliophthora thermophila), Myrothecium, Neocallimastix, Neurospora (e.g., Neurospora crassa), Paecilomyces, Penicillium (e.g., Penicillium chrysogenum, Pennicillium iilacinum, Penicillium roquefortii), Phenerochaete, Phlebia, Piromyces, Pythium, Rhizopus (e.g., Rhizopus niveus), Schizophyllum, Scytalidium, Sporotrichum (e.g., Sporotrichum cellulophilum), Stereum, Talaromyces, Thermoascus, Thermomyces, Thielavia (e.g., Thielavia terrestris), Tolypocladium, and Trichoderma (e.g., Trichoderma harzianum, Trichoderma koningii, Trichoderma longibrachiatum, Trichoderma reesei, Trichoderma atroviride, Trichoderma virens, Trichoderma citrinoviride, Trichoderma viride).

Non-limiting examples of suitable bacteria include firmicutes, cyanobacteria (blue-green algae), oscillatoriophcideae, bacillales, lactobacillales, oscillatoriales, bacillaceae, lactobacillaceae, and members of any of the following genera, and derivatives and crosses thereof: Acinetobacter, Acetobacter (e.g., Acetobacter suboxydans, Acetobacter xylinum), Actinoplane (e.g., Actinoplane missouriensis), Arthrospira (e.g., Arthrospira platensis, Arthrospira maxima), Bacillus (e.g., Bacillus cereus, Bacillus coagulans, Bacillus licheniformis, Bacillus stearothermophilus, Bacillus subtilis), Escherichia (e.g., Escherichia coli), Lactobacillus (e.g., Lactobacillus acidophilus, Lactobacillus bulgaricus), Lactococcus (e.g., Lactococcus lactis, Lactococcus lactis Lancefield Group N, Lactobacillus reuteri), Leuconostoc (e.g., Leuconostoc citrovorum, Leuconostoc dextranicum, Leuconostoc mesenteroides), Micrococcus (e.g., Micrococcus lysodeikticus), Rhodococcus (e.g., Rhodococcus opacus, Rhodococcus opacus strain PD630), Spirulina, Streptococcus (e.g., Streptococcus cremoris, Streptococcus lactis, Streptococcus lactis subspecies diacetylactis, Streptococcus thermophilus), Streptomyces (e.g., Streptomyces chattanoogensis, Streptomyces griseus, Streptomyces natalensis, Streptomyces olivaceus, Streptomyces olivochromogenes, Streptomyces rubiginosus), Tetrahymena (e.g., Tetrahymena thermophile, Tetrahymena hegewischi, Tetrahymena hyperangularis, Tetrahymena malaccensis, Tetrahymena pigmentosa, Tetrahymena pyriformis, Tetrahymena vorax), and Xanthomonas (e.g., Xanthomonas campestris).

Non-limiting examples of suitable algae include members of any of the following genera, and derivatives and crosses thereof: red algae, brown algae, gree algae, microalgae, Acinetobacter, Achnanthes (e.g., Achnanthes orientalis), Agmenellum, Alaria (e.g., Alaria marginata), Amphiprora (e.g., Amphiprora hyaline), Amphora (e.g., Amphora coffeiformis, Amphora coffeiformis linea, Amphora coffeiformis punctata, Amphora coffeiformis taylori, Amphora coffeiformis tenuis, Amphora delicatissima, Amphora delicatissima capitata, Amphora sp.), Anabaena, Analipus (e.g., Analipus japonicus), Ankistrodesmus (e.g., Ankistrodesmus falcatus), Ascophyllum (e.g., Ascophyllum nodosum), Boekelovia (e.g., Boekelovia hooglandii), Borodinella (e.g., Borodinella sp.), Botryococcus (e.g., Botryococcus braunii, Botryococcus sudeticus), Carteria, Chaetoceros (e.g., Chaetoceros gracilis, Chaetoceros muelleri, Chaetoceros muelleri subsalsum, Chaetoceros sp.), Chlorella (e.g., Chlorella anitrata, Chlorella Antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora (strain SAG 37.88), Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella parva, Chlorella photophila, Chlorella pringsheimii, Chlorella protothecoides, Chlorella protothecoides var. acidicola, Chlorealla, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris, Chlorella vulgaris f. tertia, Chlorella vulgaris var. autotrophica, Chlorella vulgaris var. viridis, Chlorella vulgaris var. vulgaris, Chlorella vulgaris var. vulgaris f. tertia, Chlorella vulgaris var. vulgaris f. viridis, Chlorella xanthella, Chlorella zofingiensis, Chlorella trebouxioides, Chlorella vulgaris), Chlorococcum (e.g., Chlorococcum infusionum, Chlorococcum sp.), Chlorogonium, Chondrus (e.g., Chondrus crispus, Chondrus ocellatus), Chroomonas (e.g., Chroomonas sp.), Chrysosphaera (e.g., Chrysosphaera sp.), Cricosphaera (e.g., Cricosphaera sp.), Cryptomonas (e.g., Cryptomonas sp.), Cyclotella (e.g., Cyclotella cryptica, Cyclotella meneghiniana, Cyclotella sp.), Dunaliella (e.g., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta), Ecklonia (e.g., Ecklonia sp), Eisenia (e.g., Eisenia bicyclis), Ellipsoidon (e.g., Ellipsoidon sp.), Eremosphaera (e.g., Eremosphaera viridis, Eremosphaera sp.), Eucheuma (e.g., Eucheuma cottonii, Eucheuma spinosum), Euglena, Fragilaria (e.g., Fragilaria crotonensis, Fragilaria sp.), Franceia (e.g., Franceia sp.), Furcellaria (e.g., Furcellaria fastigiate), Gigartina (e.g., Gigartina acicularis, Gigartina bursa-pastoris, Gigartina pistillata, Gigartina radula, Gigartina skottsbergii, Gigartina stellate), Gleocapsa (e.g., Gleocapsa sp.), Gloeothamnion (e.g., Gloeothamnion sp.), Gloiopeltis (e.g., Gloiopeltis furcate), Gracilaria (e.g., Gracilaria bursa-pastoris, Gracilaria lichenoides), Hizikia (e.g., Hizikia fusiforme), Hymenomonas (e.g., Hymenomonas sp.), Isochrysis (e.g., Isochrysis aff. galbana, Isochrysis galbana), Kjellmaniella (e.g., Kjellmaniella gyrate), Laminaria (e.g., Laminaria angustata, Laminaria longirruris, Laminaria Longissima, Laminaria ochotensis, Laminaria claustonia, Laminaria saccharina, Laminaria digitata, Laminaria japonica), Lepocinclis, Macrocystis (e.g., Macrocystis pyrifera), Micractinium, Monoraphidium (e.g., Monoraphidium minutum, Monoraphidium sp.), Nannochloris (e.g., Nannochloris sp.), Nannochloropsis (e.g., Nannochloropsis salina, Nannochloropsis sp.), Navicula (e.g., Navicula acceptata, Navicula biskanterae, Navicula pseudotenelloides, Navicula pelliculosa, Navicula saprophila, Navicula sp.), Nephrochloris (e.g., Nephrochloris sp.), Nephroselmis (e.g., Nephroselmis sp.), Nitzschia (e.g., Nitzschia communis, Nitzschia alexandrina, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp.), Ochromonas (e.g., Ochromonas sp.), Oocystis (e.g., Oocystis parva, Oocystis pusilla, Oocystis sp.), Oscillatoria (e.g., Oscillatoria limnetica, Oscillatoria sp., Oscillatoria subbrevis), Palmaria (e.g., Palmaria palmata), Pascheria (e.g., Pascheria acidophila), Pavlova (e.g., Pavlova sp.), Petalonia (e.g., Petalonia fascia), Phagus, Phormidium, Platymonas (e.g., Platymonas sp.), Pleurochrysis (e.g., Pleurochrysis carterae, Pleurochrysis dentate, Pleurochrysis sp.), Porphyra (e.g., Porphyra columbina, Porphyra crispata, Porhyra deutata, Porhyra perforata, Porhyra suborbiculata, Porphyra tenera), Porphyridium (e.g., Porphyridium cruentum, Porphyridium purpureum, Porphyridium aerugineum), Prototheca (e.g., Prototheca wickerhamii, Prototheca stagnora, Prototheca portoricensis, Prototheca moriformis, Prototheca zopfii), Pyramimonas (e.g., Pyramimonas sp.), Pyrobotrys, Rhodella (e.g., Rhodella maculate, Rhodella reticulata, Rhodella violacea), Rhodymenia (e.g., Rhodymenia palmata), Sarcinoid (e.g., Sarcinoid chrysophyte), Scenedesmus (e.g., Scenedesmus armatus), Scytosiphon (e.g., Scytosiphon lome), Spirogyra, Spirulina (e.g., Spirulina platensis), Stichococcus (e.g., Stichococcus sp.), Synechococcus (e.g., Synechococcus sp.), Tetraedron, Tetraselmis (e.g., Tetraselmis sp., Tetraselmis suecica), Thalassiosira (e.g., Thalassiosira weissflogii), and Viridiella (e.g., Viridiella fridericiana).

Non-limiting examples of suitable protozoa include but are not limited to Tetrahymena thermophile, Tetrahymena hegewischi, Tetrahymena hyperangularis, Tetrahymena malaccensis, Tetrahymena pigmentosa, Tetrahymena pyriformis, and Tetrahymena vorax.

Considerations that can affect selection of a suitable parent cell can include production of suitable lipids or hydrocarbons for production of a milk lipid or milk lipid precursor, high lipid content as a percentage of cell weight, ease of growth, ease of genetic engineering, ease of biomass processing, and heterotrophic growth (i.e., growth on sugar in absence of light). Identification of suitable sources can be done, for example, by analysis of fatty acid methyl esters produced using gas chromatography extraction and analysis or infrared measurements (see, for example, Whittaker et al., 2003. J Microbiol Methods. 55(3):709-16).

The parent cell can be an oleaginous cell (i.e., a cell that can produce and accumulate lipid to at least 20% by weight of dry cell mass). The oleaginous cell can be a native oleaginous cell (i.e., a cell that is natively oleaginous; non-limiting examples of oleaginous cells are disclosed herein), or a recombinant oleaginous cell (i.e., a cell that is rendered oleaginous via genetic engineering). The oleaginous cell can produce and accumulate lipid to at least 20%, at least 30%, at least 40%, at least 50%, at least 60%, at least 70%, or at least 75% by weight of dry cell mass. Suitable oleaginous cells can be identified by screening for production of lipids (for example, by staining using Sudan black, Nile red, or Oil Red 0, and use of high-throughput cytometry platforms), followed by thin-layer chromatography (TLC) analysis of candidate strains for lipid profiles, and GC/MS on selected candidates).

The parent cell can also be a generally recognized as safe (GRAS) host cell, such as a food-grade/edible/GRAS-certified microorganism.

### Culturing

Suitable conditions for producing a milk lipid or milk lipid precursor are typically those under which the recombinant host cell according to any of the above can grow and/or remain viable, and produce the milk lipid or milk lipid precursor.

Non-limiting examples of suitable conditions include a suitable culture medium (e.g., a culture medium having a suitable nutrient content [e.g., a suitable carbon content, a suitable nitrogen content, a suitable phosphorus content], a suitable supplement content, a suitable trace metal content, a suitable pH), a suitable temperature, a suitable feed rate, a suitable pressure, a suitable level of oxygenation, a suitable fermentation duration (i.e., volume of culture media comprising the recombinant host cells), a suitable fermentation volume (i.e., volume of culture media comprising the recombinant host cells), and a suitable fermentation vessel.

Suitable culture media include all culture media in which the recombinant host cell can grow and/or remain viable, and produce the recombinant protein. Typically, the culture medium is an aqueous medium that comprises a carbon source (i.e., a compound that comprises carbon and that can be metabolized by a host cell), an assimilable nitrogen source (i.e., a nitrogen-containing compound capable of releasing nitrogen in a form suitable for metabolic utilization by the recombinant host cell), and a phosphate source.

Non-limiting examples of carbon sources include monosaccharides, disaccharides, polysaccharides, acetate, ethanol, methanol, glycerol, methane, and combinations thereof. Non-limiting examples of monosaccharides include dextrose (glucose), fructose, galactose, xylose, arabinose, and combinations thereof. Non-limiting examples of disaccharides include sucrose, lactose, maltose, trehalose, cellobiose, and combinations thereof. Non-limiting examples of polysaccharides include starch, glycogen, cellulose, amylose, hemicellulose, maltodextrin, and combinations thereof.

Non-limiting examples of assimilable nitrogen sources include anhydrous ammonia, ammonium sulfate, ammonium hydroxide, ammonium nitrate, diammonium phosphate, monoammonium phosphate, ammonium pyrophosphate, ammonium chloride, sodium nitrate, urea, peptone, protein hydrolysates, corn steep liquor, corn steep solids, spent grain, spent grain extract, and yeast extract. Use of ammonia gas is convenient for large scale operations, and can be employed by bubbling through the aqueous ferment (fermentation medium) in suitable amounts. At the same time, such ammonia can also be employed to assist in pH control.

The culture medium can further comprise an inorganic salt, a mineral (e.g., magnesium, calcium, potassium, sodium; e.g., in suitable soluble assimilable ionic and combined forms), a metal or transition metal (e.g., copper, manganese, molybdenum, zinc, iron, boron, iodine; e.g., in suitable soluble assimilable form), a vitamin, and any other nutrient or functional ingredient (e.g., a protease [e.g., a plant-based protease] that can prevent degradation of the recombinant protein, a protease inhibitor that can reduce the activity of a protease that can degrade the recombinant protein, and/or a sacrificial protein that can siphon away protease activity, an anti-foaming agent, an anti-microbial agent, a surfactant, an emulsifying oil).

Suitable culture media are available from commercial suppliers or may be prepared according to published compositions (e.g., in catalogues of the American Type Culture Collection).

A suitable pH can be a pH of between 2 and 8, 7.5, 7, 6.5, 6, 5.5, 5.4, 5.3, 5.2, 5.1, 5, 4.9, 4.8, 4.7, 4.6, 4.5, 4, 3.5, 3, or 2.5; between 2.5 and 8, 7.5, 7, 6.5, 6, 5.5, 5.4, 5.3, 5.2, 5.1, 5, 4.9, 4.8, 4.7, 4.6, 4.5, 4, 3.5, or 3; between 3 and 8, 7.5, 7, 6.5, 6, 5.5, 5.4, 5.3, 5.2, 5.1, 5, 4.9, 4.8, 4.7, 4.6, 4.5, 4, or 3.5; between 3.5 and 8, 7.5, 7, 6.5, 6, 5.5, 5.4, 5.3, 5.2, 5.1, 5, 4.9, 4.8, 4.7, 4.6, 4.5, or 4; between 4 and 8, 7.5, 7, 6.5, 6, 5.5, 5.4, 5.3, 5.2, 5.1, 5, 4.9, 4.8, 4.7, 4.6, or 4.5; between 4.5 and 8, 7.5, 7, 6.5, 6, 5.5, 5.4, 5.3, 5.2, 5.1, 5, 4.9, 4.8, 4.7, or 4.6; between 4.6 and 8, 7.5, 7, 6.5, 6, 5.5, 5.4, 5.3, 5.2, 5.1, 5, 4.9, 4.8, or 4.7; between 4.7 and 8, 7.5, 7, 6.5, 6, 5.5, 5.4, 5.3, 5.2, 5.1, 5, 4.9, or 4.8; between 4.8 and 8, 7.5, 7, 6.5, 6, 5.5, 5.4, 5.3, 5.2, 5.1, 5, or 4.9; between 4.9 and 8, 7.5, 7, 6.5, 6, 5.5, 5.4, 5.3, 5.2, 5.1, or 5; between 5 and 8, 7.5, 7, 6.5, 6, 5.5, 5.4, 5.3, 5.2, or 5.1; between 5.1 and 8, 7.5, 7, 6.5, 6, 5.5, 5.4, 5.3, or 5.2; between 5.2 and 8, 7.5, 7, 6.5, 6, 5.5, 5.4, or 5.3; between 5.3 and 8, 7.5, 7, 6.5, 6, 5.5, or 5.4; between 5.4 and 8, 7.5, 7, 6.5, 6, or 5.5; between 5.5 and 8, 7.5, 7, 6.5, or 6; between 6 and 8, 7.5, 7, or 6.5; between 6.5 and 8, 7.5, or 7; between 7 and 8, or 7.5; or between 7.5 and 8.

A suitable temperature can be a temperature of between 20° C. and 46° C., 44° C., 42° C., 40° C., 38° C., 36° C., 34° C., 32° C., 30° C., 28° C., 26° C., 24° C., or 22° C.; between 22° C. and 46° C., 44° C., 42° C., 40° C., 38° C., 36° C., 34° C., 32° C., 30° C., 28° C., 26° C., or 24° C.; between 24° C. and 46° C., 44° C., 42° C., 40° C., 38° C., 36° C., 34° C., 32° C., 30° C., 28° C., or 26° C.; between 26° C. and 46° C., 44° C., 42° C., 40° C., 38° C., 36° C., 34° C., 32° C., 30° C., or 28° C.; between 28° C. and 46° C., 44° C., 42° C., 40° C., 38° C., 36° C., 34° C., 32° C., or 30° C.; between 30° C. and 46° C., 44° C., 42° C., 40° C., 38° C., 36° C., 34° C., or 32° C.; between 32° C. and 46° C., 44° C., 42° C., 40° C., 38° C., 36° C., or 34° C.; between 36° C. and 46° C., 44° C., 42° C., 40° C., or 38° C.; between 38° C. and 46° C., 44° C., 42° C., or 40° C.; between 40° C. and 46° C., 44° C., or 42° C., between 42° C. and 46° C. or 44° C.; or between 44° C. and 46° C.

A suitable feed rate can be a feed rate of between 0.01 g and 0.2 g glucose equivalent per g dry cell weight (DCW) per hour.

A suitable pressure can be a pressure of between 0 psig and 50 psig, 40 psig, 30 psig, 20 psig, or 10 psig; between 10 psig and 50 psig, 40 psig, 30 psig, or 20 psig; between 20 psig and 50 psig, 40 psig, or 30 psig; between 30 psig and 50 psig, or 40 psig; or between 40 psig and 50 psig.

A suitable oxygenation can be an aeration rate of between 0.1 volumes of oxygen per liquid volume in the fermentor per minute (vvm) and 2.1 vvm, 1.9 vvm, 1.7 vvm, 1.5 vvm, 1.3 vvm, 1.1 vvm, 0.9 vvm, 0.7 vvm, 0.5 vvm, or 0.3 vvm; between 0.3 vvm and 2.1 vvm, 1.9 vvm, 1.7 vvm, 1.5 vvm, 1.3 vvm, 1.1 vvm, 0.9 vvm, 0.7 vvm, or 0.5 vvm; between 0.5 vvm and 2.1 vvm, 1.9 vvm, 1.7 vvm, 1.5 vvm, 1.3 vvm, 1.1 vvm, 0.9 vvm, or 0.7 vvm; between 0.7 vvm and 2.1 vvm, 1.9 vvm, 1.7 vvm, 1.5 vvm, 1.3 vvm, 1.1 vvm, or 0.9 vvm; between 0.9 vvm and 2.1 vvm, 1.9 vvm, 1.7 vvm, 1.5 vvm, 1.3 vvm, or 1.1 vvm; between 1.1 vvm and 2.1 vvm, 1.9 vvm, 1.7 vvm, 1.5 vvm, or 1.3 vvm; between 1.3 vvm and 2.1 vvm, 1.9 vvm, 1.7 vvm, or 1.5 vvm; between 1.5 vvm and 2.1 vvm, 1.9 vvm, or 1.7 vvm; between 1.7 vvm and 2.1 vvm or 1.9 vvm; or between 1.9 vvm and 2.1 vvm.

A suitable fermentation duration can be a fermentation duration of between 10 hours and 500 hours, 400 hours, 300 hours, 200 hours, 100 hours, 50 hours, 40 hours, 30 hours, or 20 hours; between 20 hours and 500 hours, 400 hours, 300 hours, 200 hours, 100 hours, 50 hours, 40 hours, or 30 hours; between 30 hours and 500 hours, 400 hours, 300 hours, 200 hours, 100 hours, 50 hours, or 40 hours; between 40 hours and 500 hours, 400 hours, 300 hours, 200 hours, 100 hours, or 50 hours; between 50 hours and 500 hours, 400 hours, 300 hours, 200 hours, or 100 hours; between 100 hours and 500 hours, 400 hours, 300 hours, or 200 hours; between 200 hours and 500 hours, 400 hours, or 300 hours; between 300 hours and 500 hours, or 400 hours; or between 400 hours and 500 hours.

A suitable fermentation volume can be between 1 L and 10,000,000 L, 5,000,000 L, 1,000,000 L, 500,000 L, 100,000 L, 50,000 L, 10,000 L, 5,000 L, 1,000 L, 500 L, 100 L, 50 L, or 10 L; between 10 L and 10,000,000 L, 5,000,000 L, 1,000,000 L, 500,000 L, 100,000 L, 50,000 L, 10,000 L, 5,000 L, 1,000 L, 500 L, 100 L, or 50 L; between 50 L and 10,000,000 L, 5,000,000 L, 1,000,000 L, 500,000 L, 100,000 L, 50,000 L, 10,000 L, 5,000 L, 1,000 L, 500 L, or 100 L; between 100 L and 10,000,000 L, 5,000,000 L, 1,000,000 L, 500,000 L, 100,000 L, 50,000 L, 10,000 L, 5,000 L, 1,000 L, or 500 L; between 500 L and 10,000,000 L, 5,000,000 L, 1,000,000 L, 500,000 L, 100,000 L, 50,000 L, 10,000 L, 5,000 L, or 1,000 L; between 1,000 L and 10,000,000 L, 5,000,000 L, 1,000,000 L, 500,000 L, 100,000 L, 50,000 L, 10,000 L, or 5,000 L; between 5,000 L and 10,000,000 L, 5,000,000 L, 1,000,000 L, 500,000 L, 100,000 L, 50,000 L, or 10,000 L; between 10,000 L and 10,000,000 L, 5,000,000 L, 1,000,000 L, 500,000 L, 100,000 L, or 50,000 L; between 50,000 L and 10,000,000 L, 5,000,000 L, 1,000,000 L, 500,000 L, or 100,000 L; between 100,000 L and 10,000,000 L, 5,000,000 L, 1,000,000 L, or 500,000 L; between 500,000 L and 10,000,000 L, 5,000,000 L, or 1,000,000 L; between 1,000,000 L and 10,000,000 L, or 5,000,000 L; or between 5,000,000 L and 10,000,000 L.

A suitable fermentation vessel can be any fermentation vessel known in the art. Non-limiting examples of suitable fermentation vessels include culture plates, shake flasks, fermentors (e.g., stirred tank fermentors, airlift fermentors, bubble column fermentors, fixed bed bioreactors, laboratory fermentors, industrial fermentors, or any combination thereof), used at any suitable scale (e.g., small-scale, large-scale) and in any process (e.g., solid culture, submerged culture, batch, fed-batch, or continuous-flow).

### Purification and Post-Processing

Methods for purifying lipids are well-known in the art, and can be adapted to purify milk lipids or milk lipid precursors produced by a recombinant host cell according to any of the above. Some such methods involve fractionation based on differing molecular weights, melting points, solubilities, and/or volatilities of molecules (e.g., lipids). A commonly practiced form of fractionation is that of crystallization wherein a mixture of molecules (e.g., lipids) is separated into two or more different fractions based on melting at a given temperature. For example, fatty acids having larger carbon atom numbers tend to have higher melting points than fatty acids having smaller carbon atom numbers, and saturated fatty acids have higher melting points than unsaturated fatty acids of the same carbon atom number. Non-limiting examples of fractionation processes include dry fractionation (e.g., winterizing, dewaxing, pressing, fractionation from melt), solvent fractionation (using, for example, acetone, ethanol, pentane, supercritical carbon dioxide), fractional chromatography, and distillation. Fractionation typically involves a filtration step in which melted, solubilized, or volatilized phases are separated from other phases (e.g., melted or solubilized phase separated from solid phase, volatilized phase separated from liquid phase). Non-limiting examples of filtration processes include vacuum filtration, pressure filtration, and centrifugation. Solvent fractionation typically further necessitates a step in which the solvent is removed.

A milk lipid or milk lipid precursor can be purified to a purity of greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 97%, or greater than 99% relative to other components comprised in the fermentation broth, or to at least 2-fold, at least 3-fold, at least 4-fold, at least 5-fold, at least 6-fold, at least 7-fold, at least 8-fold, at least 9-fold, or at least 10-fold greater abundancy relative to other components comprised in the fermentation broth, or to a purity of greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 97%, or greater than 99% by weight.

The milk lipid or milk lipid precursor can be spray dried or concentrated via evaporation (e.g., to obtain a powder).

### Method of Producing Food Product

In another aspects, provided herein is a method for producing the food product according to any of the above.

A variety of recipes exist for preparing a food product, and any such recipe can be used to produce a food product according to any of the above. The lipid component according to any of the above can be used in such recipes in place of other lipids (e.g., milk fat) and conventionally used food ingredients. Alternatively, milk lipids, and optionally non-milk lipids, can be individually added to produce the food product.

Some food products require fermentation by microbial cells (e.g., lactic acid bacteria, fungal cells [e.g., yeast, filamentous fungal cells, mold]) for texture and/or flavor production. Such food products can be produced using the lipid component according to any of the above in place of conventionally used substrates (e.g., milk fat). Non-limiting examples of such food products include sour cream, cottage cheese, buttermilk, yogurt, and ripened cheese.

What is claimed is:

1. A lipid component, wherein the lipid component consists of a milk lipid component and an optional non-milk lipid component.

2. The lipid component of claim 1, wherein the milk lipid component consists of a single milk lipid.

3. The lipid component of claim 2, wherein the single milk lipid is a mTAG.

4. The lipid component of claim 2, wherein the single milk lipid is a mDAG.

5. The lipid component of claim 2, wherein the single milk lipid is a mMAG.

6. The lipid component of claim 2, wherein the single milk lipid is a mPL.

7. The lipid component of claim 2, wherein the single milk lipid is a milk sterol.

8. The lipid component of claim 2, wherein the single milk lipid is a mFFA.

9. The milk lipid component of claim 1, wherein the milk lipid component consists of two or more distinct milk lipids.

10. The milk lipid component of claim 9, wherein the two or more distinct milk lipids comprise a mTAG.

11. The milk lipid component of claim 9, wherein the two or more distinct milk lipids comprise a mDAG.

12. The milk lipid component of claim 9, wherein the two or more distinct milk lipids comprise a mMAG.

13. The milk lipid component of claim 9, wherein the two or more distinct milk lipids comprise a mPL.

14. The milk lipid component of claim 9, wherein the two or more distinct milk lipids comprise a milk sterol.

15. The milk lipid component of claim 9, wherein the two or more distinct milk lipids comprise a mFFA.

16. The lipid component of claim 3 or 10, wherein the mTAG comprises a fatty acid in sn1, sn2, and/or sn3 position that has a carbon atom number that ranges from 4 to 24.

17. The lipid component of claim 16, wherein the fatty acid is selected from the group consisting of butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

18. The milk lipid component of claim 3 or 10, wherein the mTAG comprises a single fatty acid having a carbon atom number that ranges from 4 to 24.

19. The milk lipid component of claim 18, wherein the single fatty acid is saturated.

20. The milk lipid component of claim 3 or 10, wherein the mTAG comprises two fatty acids each having a carbon atom number that ranges from 4 to 24.

21. The milk lipid component of claim 20, wherein one of the two fatty acids is saturated.

22. The milk lipid component of claim 20, wherein both of the two fatty acids are saturated.

23. The milk lipid component of claim 3 or 10, wherein the mTAG comprises three fatty acids each having a carbon atom number that ranges from 4 to 24.

24. The lipid component of claim 23, wherein one of the three fatty acids is saturated.

25. The lipid component of claim 23, wherein two of the three fatty acids are saturated.

26. The lipid component of claim 23, wherein all of the three fatty acids are saturated.

27. The lipid component of claim 3 or 10, wherein the mTAG comprises a fatty acid in sn3 position that is saturated and has a carbon atom number that ranges from 4 to 10.

28. The lipid component of claim 27, wherein the fatty acid is selected from the group consisting of butyric acid (C4:0), caproic acid (C6:0), and caprylic acid (C8:0).

29. The lipid component of claim 3 or 10, wherein the mTAG comprises a fatty acid in sn2 position that is saturated and has a carbon atom number that ranges from 8 to 16.

30. The milk lipid component of claim 29, wherein the fatty acid is selected from the group consisting of caprylic acid (C8:0), capric acid (C10:0), lauric acid (C12:0), myristic acid (C14:0), pentadecanoic acid (C15:0), and palmitic acid (C16:0).

31. The lipid component of claim 3 or 10, wherein the mTAG comprises a fatty acid in sn1 and/or sn3 position that has a carbon atom number that ranges from 18 to 24.

32. The lipid component of claim 31, wherein the fatty acid is selected from the group consisting of stearic acid (C18:0) and oleic acid (C18:1).

33. The lipid component of claim 3 or 10, wherein the mTAG has a first fatty acid in sn1 position that has a carbon atom number that ranges from 16 to 54, and a second fatty acid in sn2 position that is saturated and has a carbon atom number that ranges from 8 to 16.

34. The lipid component of claim 33, wherein the first fatty acid is selected from the group consisting of palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11); and wherein the second fatty acid is selected from the group consisting of caprylic acid (C8:0), capric acid (C10:0), lauric acid (C12:0), myristic acid (C14:0), pentadecanoic acid (C15:0), palmitic acid (C16:0), and palmitoleic acid (C16:1).

35. The lipid component of claim 3 or 10, wherein the mTAG has a first fatty acid in sn1 position that has a carbon atom number that ranges from 16 to 54, and a second fatty acid in sn3 position that has a carbon atom number that ranges from 4 to 10.

36. The lipid component of claim 35, wherein the first fatty acid is selected from the group consisting of palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11); and wherein the second fatty acid is selected from the group consisting of butyric acid (C4:0), caproic acid (C6:0), caprylic acid (C8:0), and capric acid (C10:0).

37. The lipid component of claim 3 or 10, wherein the mTAG has a first fatty acid in sn2 position that has a carbon atom number that ranges from 8 to 16, and a second fatty acid in sn3 position that has a carbon atom number that ranges from 4 to 10.

38. The lipid component of claim 37, wherein the first fatty acid is selected from the group consisting of caprylic acid (C8:0), capric acid (C10:0), lauric acid (C12:0), myristic acid (C14:0), pentadecanoic acid (C15:0), palmitic acid (C16:0), and palmitoleic acid (C16:1); and wherein the second fatty acid is selected from the group consisting of butyric acid (C4:0), caproic acid (C6:0), caprylic acid (C8:0), and capric acid (C10:0).

39. The lipid component of claim 3 or 10, wherein the mTAG has a first fatty acid in sn2 position that has a carbon atom number that ranges from 16 to 54, and a second fatty acid in sn3 position that has a carbon atom number that ranges from 4 to 10.

40. The lipid component of claim 39, wherein the first fatty acid is selected from the group consisting of palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11); and wherein the second fatty acid is selected from the group consisting of butyric acid (C4:0), caproic acid (C6:0), caprylic acid (C8:0), and capric acid (C10:0).

41. The lipid component of claim 3 or 10, wherein the mTAG has a first fatty acid in sn1 position that has a carbon atom number that ranges from 8 to 16, and a second fatty acid in sn3 position that has a carbon atom number that ranges from 4 to 10.

42. The lipid component of claim 41, wherein the first fatty acid is selected from the group consisting of caprylic acid (C8:0), capric acid (C10:0), lauric acid (C12:0), myristic acid (C14:0), pentadecanoic acid (C15:0), palmitic acid (C16:0), and palmitoleic acid (C16:1); and the second fatty acid is selected from the group consisting of butyric acid (C4:0), caproic acid (C6:0), caprylic acid (C8:0), and capric acid (C10:0).

43. The lipid component of claim 3 or 10, wherein the mTAG has a first fatty acid in sn1 position that has a carbon atom number that ranges from 16 to 54, a second fatty acid in sn2 position that has a carbon atom number that ranges from 8 to 16, and a third fatty acid in sn3 position that has a carbon atom number that ranges from 4 to 10.

44. The lipid component of claim 43, wherein the first fatty acid is selected from the group consisting of palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11); the second fatty acid is selected from the group consisting of caprylic acid (C8:0), capric acid (C10:0), lauric acid (C12:0), myristic acid (C14:0), pentadecanoic acid (C15:0), palmitic acid (C16:0), and palmitoleic acid (C16:1); and the third fatty acid is selected from the group consisting of butyric acid (C4:0), caproic acid (C6:0), caprylic acid (C8:0), and capric acid (C10:0).

45. The lipid component of claim 3 or 10, wherein the mTAG has a first fatty acid in sn1 position that has a carbon atom number that ranges from 16 to 54, a second fatty acid in sn2 position that has a carbon atom number that ranges from 16 to 54, and a third fatty acid in sn3 position that has a carbon atom number that ranges from 4 to 10.

46. The lipid component of claim 45, wherein the first fatty acid and the second fatty acid are independently selected from the group consisting of palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11); and the third fatty acid is selected from the group consisting of butyric acid (C4:0), caproic acid (C6:0), caprylic acid (C8:0), and capric acid (C10:0).

47. The lipid component of claim 3 or 10, wherein the mTAG has a first fatty acid in sn1 position that has a carbon atom number that ranges from 8 to 16, a second fatty acid in sn2 position that has a carbon atom number that ranges from 16 to 54, and a third fatty acid in sn3 position that has a carbon atom number that ranges from 4 to 10.

48. The lipid component of claim 47, wherein the first fatty acid is selected from the group consisting of caprylic acid (C8:0), capric acid (C10:0), lauric acid (C12:0), myristic acid (C14:0), pentadecanoic acid (C15:0), palmitic acid (C16:0), and palmitoleic acid (C16:1); the second fatty acid is selected from the group consisting of palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11); and the third fatty acid is selected from the group consisting of butyric acid (C4:0), caproic acid (C6:0), caprylic acid (C8:0), and capric acid (C10:0).

49. The lipid component of claim 3 or 10, wherein the mTAG has a first fatty acid in sn1 position that has a carbon atom number that ranges from 8 to 16, a second fatty acid in sn2 position that has a carbon atom number that ranges from 8 to 16, and a third fatty acid in sn3 position that has a carbon atom number that ranges from 4 to 10.

50. The lipid component of claim 49, wherein the first fatty acid and the second fatty acid are independently selected from the group consisting of caprylic acid (C8:0), capric acid (C10:0), lauric acid (C12:0), myristic acid (C14:0), pentadecanoic acid (C15:0), palmitic acid (C16:0), and palmitoleic acid (C16:1); and the third fatty acid is selected from the group consisting of butyric acid (C4:0), caproic acid (C6:0), caprylic acid (C8:0), and capric acid (C10:0).

51. The lipid component of claim 3 or 10, wherein the mTAG has a first fatty acid in sn1 position that has a carbon atom number that ranges from 16 to 54, a second fatty acid in and sn2 position that has a carbon atom number that ranges from 16 to 54, and a third fatty acid in sn3 position that has a carbon atom number that ranges from 8 to 16.

52. The lipid component of claim 51, wherein the first fatty acid and the second fatty acid are independently selected from the group consisting of palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11); and the third fatty acid is selected from the group consisting of caprylic acid (C8:0), capric acid (C10:0), lauric acid (C12:0), myristic acid (C14:0), pentadecanoic acid (C15:0), palmitic acid (C16:0), and palmitoleic acid (C16:1).

53. The lipid component of claim 3 or 10, wherein the mTAG has a first fatty acid in sn2 position that each has a carbon atom number that ranges from 8 to 16, a second fatty acid in sn3 position that has a carbon atom number that ranges from 8 to 16, and a third fatty acid in sn1 position that has a carbon atom number that ranges from 16 to 54.

54. The lipid component of claim 53, wherein the first fatty acid and the second fatty acid are independently selected from the group consisting of caprylic acid (C8:0), capric acid (C10:0), lauric acid (C12:0), myristic acid (C14:0), pentadecanoic acid (C15:0), palmitic acid (C16:0), and palmitoleic acid (C16:1); and the third fatty acid is selected from the group consisting of palmitic acid (C16:0), and palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

55. The lipid component of claim 3 or 10, wherein the mTAG has a first fatty acid in sn1 position that has a carbon atom number that ranges from 16 to 54, a second fatty acid in sn2 position that has a carbon atom number that ranges from 4 to 10, and a third fatty acid in sn3 position that has a carbon atom number that ranges from 8 to 16.

56. The lipid component of claim 55, wherein the first fatty acid is selected from the group consisting of palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11); the second fatty acid is selected from the group consisting of butyric acid (C4:0), caproic acid (C6:0), caprylic acid (C8:0), and capric acid (C10:0); and the third fatty acid is selected from the group consisting of caprylic acid (C8:0), capric acid (C10:0), lauric acid (C12:0), myristic acid (C14:0), pentadecanoic acid (C15:0), palmitic acid (C16:0), and palmitoleic acid (C16:1).

57. The lipid component of claim 3 or 10, wherein the mTAG has a first fatty acid in sn1 position that has a carbon atom number that ranges from 16 to 54, a second fatty acid in sn2 position that has a carbon atom number that ranges from 4 to 10, and a third fatty acid in sn3 position that has a carbon atom number that ranges from 4 to 10.

58. The lipid component of claim 57, wherein the first fatty acid is selected from the group consisting of palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11); and the second fatty acid and the third fatty acid are independently selected from the group consisting of butyric acid (C4:0), caproic acid (C6:0), caprylic acid (C8:0), and capric acid (C10:0).

59. The lipid component of claim 3 or 10, wherein the milk lipid component consists of one or more mTAGs having a structure selected from the group consisting of C16:0-C14:0-C4:0, C14:0-C16:0-C18:1, C16:0-C16:0-C18:1, C16:0-C16:0-C4:0, C18:1-C16:0-C4:0, C18:1(n-9)-C16:0-C14:0, C18:1(n-9)-C16:0-C16:0, C18:1(n-9)-C16:0-C4:0, C16:0-C18:1-C18:1, C4:0-C14:0-C16:0, C16:0-C16:0-C16:1, C4:0-C16:0-C16:0, C4:0-C16:0-C18:0, C4:0-C16:0-C18:1, C6:0-C14:0-C16:0, and C14:0-C18:0-C18:1.

60. The lipid component of claim 8 or 15, wherein the mFFA has a carbon atom number that ranges from 4 to 24.

61. The lipid component of claim 8 or 15, wherein the mFFA is selected from the group consisting of: butyric acid (C4:0), caproic acid (C6:0), caprylic acid (C8:0), capric acid (C10:0), lauric acid (C12:0), myristic acid (C14:0), pentadecanoic acid (C15:0), palmitic acid (C16:0), palmitoleic acid (C16:1), margaric acid (C17:0), stearic acid (C18:0), oleic acid (C18:1), linoleic acid (C18:2), linolenic acid (C18:3), and vaccenic acid (C18:1 trans11).

62. The lipid component of claim 1, wherein the milk lipid component is essentially free of one or more fatty acids having a carbon atom number of greater than 24.

63. The lipid component of claim 1, wherein the milk lipid component comprises one or more milk lipids at relative ratios found in a mammal-produced milk or milk fat.

64. The milk lipid component of claim 1, wherein the milk lipid component comprises one or more milk lipids at relative ratios not found in a mammal-produced milk or milk fat.

65. The lipid component of claim 1, wherein the milk lipid component has a fatty acid profile that is identical or similar to the fatty acid profile of a mammal-produced milk or milk fat.

66. The lipid component of claim 1, wherein the milk lipid component has a fatty acid profile that is different from the fatty acid profile of a mammal-produced milk or milk fat.

67. The lipid component of claim 1, wherein the milk lipid component comprises between 0% and 50% by mass of butyric acid (C4:0), between 0% and 50% by mass of caproic acid (C6:0), between 0% and 50% by mass of caprylic acid (C8:0), between 0% and 50% by mass of capric acid (C10:0), between 0% and 50% by mass of lauric acid (C12:0), between 0% and 50% by mass of myristic acid (C14:0), between 0% and 50% by mass of pentadecanoic acid (C15:0), between 0% and 50% by mass of palmitic acid (C16:0), between 0% and 50% by mass of palmitoleic acid (C16:1), between 0% and 50% by mass of margaric acid (C17:0), between 0% and 50% by mass of stearic acid (C18:0), and/or between 0% and 50% by mass of oleic acid (C18:1).

68. The lipid component of claim 1, wherein the lipid component consists of a low-melting fraction.

69. The lipid component of claim 1, wherein the lipid component consists of a medium-melting fraction.

70. The lipid component of claim 1, wherein the lipid component consists of a high-melting melting fraction.

71. The lipid component of claim 1, wherein the lipid component consists of a low-melting fraction and a medium-melting fraction.

72. The lipid component of claim 1, wherein the lipid component consists of a low-melting fraction and a high-melting fraction.

73. The lipid component of claim 1, wherein the lipid component consists of a medium-melting fraction and a high-melting fraction.

74. The lipid component of claim 1, wherein the lipid component consists of a low-melting fraction, a medium-melting fraction, and a high-melting fraction.

75. The lipid component of claim 1, wherein the lipid component comprises solid lipid at ambient temperature and conditions.

76. The lipid component of claim 1, wherein the lipid component comprises solid lipid at body temperature and conditions.

77. The lipid component of claim 1, wherein the lipid component has a flavor/aroma profile that is similar to that of a mammal-produced milk fat.

78. The lipid component of claim 1, wherein the lipid component has a flavor/aroma profile that is bland.

79. The lipid component of claim 1, wherein the lipid component has an emulsifying potential that is similar to that of a mammal-produced milk fat.

80. The lipid component of claim 1, wherein the lipid component has an emulsifying potential that is greater than that of soybean oil.

81. The lipid component of claim 1, wherein the lipid component comprises between 0.001% and 100% by mass of the milk lipid component.

82. The lipid component of claim 1, wherein the lipid component comprises between 0.01% and 90% by mass of the optional non-milk lipid component.

83. The lipid component of claim 1, wherein the lipid component comprises the milk lipid component and the optional non-milk lipid component at a mass ratio of between 100 to 1 and 1 to 100.

84. A composition comprising the lipid component of claim 1, wherein the composition comprises no other lipid than the lipids of which the lipid component consists.

85. The composition of claim 84, wherein the composition further comprises a milk protein component.

86. The composition of claim 85, wherein the milk protein component consists of a single milk protein.

87. The composition of claim 86, wherein the single milk protein is selected from the group consisting of β-lactoglobulin, α-lactalbumin, κ-casein, β-casein, and γ-casein.

88. The composition of claim 85, wherein the milk protein component consists of a two or more milk proteins.

89. The composition of claim 88, wherein the two or more milk proteins are selected from the group consisting of two or more whey proteins, two or more caseins, and a mixture of one or more whey proteins and one or more caseins.

90. The composition of claim 89, wherein the whey proteins are selected from the group consisting of β-lactoglobulin, α-lactalbumin, serum albumin, immunoglobulins, lactoferrin, glycomacropeptide, and transferrin.

91. The composition of claim 89, wherein the casein proteins are selected from the group consisting of κ-casein, β-casein, γ-casein, α-S1-casein, and α-S2-casein.

92. The composition of claim 85, wherein the milk protein component consists of or comprises a recombinant milk protein.

93. The composition of claim 92, wherein the recombinant milk protein has a non-mammalian post-translational modification (PTM).

94. The composition of claim 84, wherein the composition further comprises a non-milk protein component.

95. The composition of claim 84, wherein the composition comprises a milk protein component and a non-milk protein component at a mass ratio of between about 100 to 1 and about 1 to 100.

96. The composition of claim 84, wherein the composition comprises a milk fat globule-like structure component.

97. The composition of claim 96, wherein the milk fat globule-like structure component comprises milk globule-like structures having an average diameter of between 0.2 μm and 15 μm.

98. The composition of claim 96, wherein the milk fat globule-like structure component comprises milk globule-like structures that comprise a milk lipid and a milk protein.

99. The composition of claim 84, wherein the composition is a powder.

100. The composition of claim 84, wherein the composition is an emulsion.

101. The composition of claim 100, wherein the emulsion comprises dispersed phase droplets having an average diameter of between 0.1 μm and 15 μm.

102. The composition of claim 100, wherein the emulsion comprises dispersed phase droplets that are engulfed in a membrane.

103. The composition of claim 84, wherein the composition comprises a flavor/aroma agent.

104. The composition of claim 103, wherein the flavor/aroma agent is a milk volatile organic compound.

105. The composition of claim 103, wherein the flavor/aroma agent is a green leaf volatile organic compound.

106. The composition of claim 105, wherein the green leaf volatile organic compound is obtained by chemical and/or enzymatic degradation of linoleic acid or linolenic acid.

107. The composition of claim 105, wherein the green leaf volatile organic compound is selected from the group consisting of hexanal, (Z)-3-hexenyl acetate, (Z)-3-hexenal, (Z)-3-hexenol, (Z)-2-hexenol, (E)-3-hexenol, (E)-2-hexenol, (E)-2-hexenal, (Z)-3-nonenol, €-2-nonenol, and 2,4-decadienal.

108. The composition of claim 84, wherein the composition is a food product.

109. The composition of claim 108, wherein the food product is selected from the group consisting of an egg, an egg product, an egg substitute, an egg product substitute, a milk, a dairy product, a milk substitute, a dairy product substitute, an animal meat, an animal meat product, an animal meat substitute, and an animal meat product substitute.

110. A method for producing the lipid component of claim 1, wherein the method comprises the step of obtaining a milk lipid or milk lipid precursor.

111. The method of claim 110, wherein the milk lipid or milk lipid precursor is obtained by production in a recombinant host cell.

112. The method of claim 111, wherein the production in a recombinant host cell comprises the step of culturing a recombinant host cell capable of producing the milk lipid or milk lipid precursor under conditions suitable for production of the milk lipid or milk lipid precursor.

113. The method of claim 110, wherein the milk lipid is obtained by chemical or enzymatic modification of a milk lipid precursor.

114. The method of claim 113, wherein the chemical or enzymatic modification of the milk lipid precursor comprises chemical or enzymatic inter-esterification.

115. The method of claim 114, wherein the enzymatic inter-esterification is accomplished with a lipase that has selectivity for a fatty acid having a carbon atom number of between 4 and 24.

116. A recombinant host cell capable of producing a milk lipid or a milk lipid precursor, wherein the recombinant host cell comprises a genetic modification that essentially eliminates or modulates production and/or activity of a lipid biosynthesis-related protein.

117. The recombinant host cell of claim 116, wherein the lipid biosynthesis-related protein is selected from the group consisting of:
a. enzymes with activity in the production of unsaturated fatty acids;
b. enzymes with activity in the production of fatty acids having a carbon atom number of greater than 16;
c. enzymes with activity in the production of fatty acids having a carbon atom number of 16 or less;
d. enzymes with activity in the b-oxidation pathway or peroxisome biogenesis;
e. enzymes with activity in the production of cytosolic acetyl-CoA;
f. enzymes with activity in the production of a TAG, DAG, MAG, and/or PL;
g. enzymes with activity in the production of an amino acid;
h. enzymes with activity in the production of cytosolic NADPH;
i. enzymes with activity in inter-esterification or trans-esterification; and
j. combinations thereof.

118. The recombinant host cell of claim 116, wherein the genetic modification consists of a single genetic modification.

119. The recombinant host cell of claim 116, wherein the genetic modification consists of two or more genetic modifications.

120. The recombinant host cell of claim 116, wherein the lipid biosynthesis-related protein consists of a single lipid biosynthesis-related protein.

121. The recombinant host cell of claim 116, wherein the lipid biosynthesis-related protein consists of two or more lipid biosynthesis-related proteins.

122. The recombinant host cell of claim 116, wherein the recombinant host cell is a recombinant microbial cell.

123. The recombinant host cell of claim 122, wherein the recombinant microbial cell in a recombinant fungal cell.

124. The recombinant host cell of claim 123, wherein the recombinant fungal cell is a recombinant yeast cell.

125. The recombinant host cell of claim 123, wherein the recombinant fungal cell is a recombinant filamentous fungal cell.

126. The recombinant host cell of claim 122, wherein the recombinant microbial cell is a recombinant bacterial cell.

127. The recombinant host cell of claim 116, wherein the recombinant host cell is an oleaginous cell.

128. A method for producing a food product, wherein the method comprises the step of combining the lipid component of claim 1 with other ingredients.

129. The method of claim 128, wherein the method comprises the step of fermenting the lipid component of claim 1 using a microbial cell.
